(19) United States
(12) Patent Application Publication
(10) Pub. No.: US 2004/0176272 A1
(43) Pub. Date: Sep. 9, 2004

(54) Uses of integrin alphavbeta3 antagonists

(75) Inventors: Ronald L. Wilder, Derwood, MD (US); Su-Yau Mao, Gaithersburg, MD (US)

Correspondence Address: JONES DAY, 222 EAST 41ST ST, NEW YORK, NY, 10017, US

(73) Assignee: MedImmune, Inc.

(21) Appl. No.: 10/769,720

(22) Filed: Jan. 30, 2004

Related U.S. Application Data
(60) Provisional application No. 60/444,156, filed on Jan. 30, 2003.

(51) Int. Cl. A61K 38/00

(52) U.S. Cl. 514/2

(57) ABSTRACT

The present invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or conditions associated therewith, utilizing antagonists of integrin αᵥβ₃. The present invention encompasses the use of methods of preventing, treating, managing or ameliorating periodontal disease, Gorham-Stout disease, Wilson's disease, chronic otitis media, hypertrophic pulmonary osteoarthropathy, or aseptic loosening of joint replacement (prostheses), or conditions associated therewith, utilizing an integrin αᵥβ₃ antagonist in combination with another therapy (e.g., another prophylactic or therapeutic agent). In particular, the present invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or conditions associated therewith, comprising administering to a subject in need thereof at least one antagonist of integrin αᵥβ₃ and at least one other therapy. The present invention encompasses compositions and articles of manufacture for use in preventing, treating, managing or ameliorating periodontal disease, or aseptic loosening of joint replacement (prostheses), Gorham-Stout disease, Wilson's disease, chronic otitis media, hypertrophic pulmonary osteoarthropathy or conditions associated therewith.

This application is entitled to and claims priority benefit of U.S. Provisional Application No. 60/444,156, filed Jan. 30, 2003, which is incorporated herein by reference in its entirety.

1. Field of the Invention

The present invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or conditions associated therewith, utilizing antagonists of integrin αᵥβ₃. The present invention encompasses the use of methods of preventing, treating, managing or ameliorating periodontal disease, Gorham-Stout disease, Wilson's disease, chronic otitis media, hypertrophic pulmonary osteoarthropathy, or aseptic loosening of joint replacement (prostheses), or conditions associated therewith, utilizing an integrin αᵥβ₃ antagonist in combination with another therapy (e.g., another prophylactic or therapeutic agent). In particular, the present invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or conditions associated therewith, comprising administering to a subject in need thereof an antagonist of integrin αᵥβ₃ and at least one other therapy. The present invention encompasses compositions and articles of manufacture for use in preventing, treating, managing or ameliorating periodontal disease, or aseptic loosening of joint replacement (prostheses), Gorham-Stout disease, Wilson's disease, chronic otitis media, hypertrophic pulmonary osteoarthropathy or conditions associated therewith.

2. Background of the Invention

2.1 Periodontal Disease

Periodontal disease is a widespread medical problem, with the majority of adults in the U.S. showing some signs or symptoms of the disease by their mid-30s. (Oral Health in America: A Report of the Surgeon General—Executive Summary. Rockville, Md.: US Department of Health and Human Services, National Institute of Dental and Craniofacial Research, National Institutes of Health, 2000, herein “Report of the Surgeon General 2000”). Periodontal disease begins with gingivitis, an inflammation of the gums characterized erythematous (redness), and edematous (swelling), bleeding, and often sensitivity and tenderness of the gingiva. (Report of the Surgeon General 2000; see also U.S. Pat. No. 6,277,587). These symptoms are a result of an accumulation of biofilm along the gingival margins and the immune system's inflammatory response to the release of destructive bacterial products. (Report of the Surgeon General 2000; see also U.S. Pat. No. 6,277,587).

The early stages of gingivitis are often reversible with thorough toothbrushing and flossing to reduce plaque. However, periodontal disease progression can not always be arrested by personal oral hygiene procedures, leading to periodontitis, i.e., tooth attachment loss. (Report of the Surgeon General 2000).

Periodontitis, in its most common form, is described as general and moderately progressing. A second, more aggressive form, is described as rapidly progressing and severe, and is often resistant to treatment. The moderately progressive adult form is characterized by a gradual loss of attachment of the periodontal ligament to the gingiva and bone along with loss of the supporting bone structure. (Report of the Surgeon General 2000). This destruction of periodontal ligament and bone gives rise to the formation of a pocket between the tooth and adjacent tissues, which harbors gingival plaque. Calculus then forms in the pocket due to inflammatory fluids and minerals in adjacent tissues and can be especially damaging. (Report of the Surgeon General 2000).

Traditional treatment for periodontal disease includes methods of increasing fibroblast attachment to root surfaces such as CO₂ laser treatment, scaling, and root planing (Crespi, R. et al., 2002, J. Periodontol 73(11):1308-12). Other treatments can be divided into two groups, antibiotics and host modulators. Host modulators include Periostat™, non-steroidal anti-inflammatory agents (NSAIDs), alendronate (Fosamax™), hormone replacement therapy and anti-arthritic medications. (Ciancio, S. G., 2002, J Clin Periodontol 29 Suppl 2:17-21; and Soory, M., 2002, Curr. Drug Targets Immune Endocr. Metabol. Disord. 2(1):13-25). NSAIDs have been used as an adjunct to mechanical removal of bacterial antigen in the management of periodontal disease. Another emerging option is surgical intervention including autogenous bone marrow stem cell transplantation with guided tissue regeneration (Ou, L., 2002, Zhong Kuo Qiang Yi Xue Za Zhi 37(2):132-4).

The chronic nature of periodontal disease, especially periodontitis, and the absence of a generally accepted animal or in vitro model, have made the molecular pathogenesis of this disease difficult to study and methods of treatment difficult to fashion. Thus, there remains a need for more efficacious therapies for the treatment of periodontal disease.

2.2 Aseptic Joint Loosening

One of the primary difficulties with joint replacement procedures is that over time, these devices tend to loosen in situ. Around 20% of total hip replacements eventually fail, with aseptic loosening being the major cause. El-Warrak, A. et al., “A New Animal Model for Aseptic Loosening in Cement Hip Replacements”, Abstract accepted for Mar. 14, 2003 Swiss Society of Biomaterials (SSB) Meeting. A chronic inflammatory reaction between the bone and the cement is usually associated with this condition resulting in an interface membrane of fibrous tissue. Among the causes attributed to trigger the formation of the interface membrane are: micromotion, wear particles, matrix degrading metalloproteinases and local inflammatory mediators (prostaglandin E2 and nitric oxide). El-Warrak et al., Id. Micromotion at the bone and cement interface is believed to cause the formation of the interface tissue. El-Warrak et al., Id. The interface membrane is believed to trigger the pathological bone resorption increasing further instability of the hip prosthesis. El-Warrak et al., Id. Formation of wear particles aggravates the inflammatory reaction and finally promotes the loosening of the prostheses component. El-Warrak et al., Id. This loosening may give rise to numerous complications which may include pain (especially transitory thigh pain), discomfort, osteoarthritis, rheumatoid arthritis, osteonecrosis, developmental dysplasia, decreased range of motion, joint function, stability and strength, and progressive joint and soft tissue deterioration. (Kim, Y. H. et al., 2003, J. Bone Joint Surg. Am 85-A(1):109-14). Left untreated, aseptic loosening may eventually result in joint dislocation, necessitating painful and costly corrective or reconstructive surgery, or as a last resort arthrodesis, surgical fusion of the joint bones (Widel, J. D., 2002, Clin. Orthop. 404:139-42).

A primary objective in joint replacement is to extend longevity through improved fixation and decreased wear and osteolysis. Current methods of treatment include pharmacological approaches: NSAIDs; simultaneous suppression of proinflammatory cytokines and PGE(2) (Lavigne, P. et al., 2002, Osteoarthritis Cartilage 10(11):898-904); oral bisphosphonate and calcium (Soininvaara, T. A., 2002, Calcif. Tissue Int. 71(6):472-7); and others such as control of biosynthesis of nitric oxide (a molecule which can activate bone resorption) (Stea, S. et al., 2002, Biomaterials 23(24):4822-8); osteoprotegerin (a natural negative regulator of osteoclastogenesis and bone resorption), and gene transfer (Yang, S. Y., et al., 2002, Arthritis Rheum. 46(9):2514-23; and Ulrich-Vinther, M., et al., 2002, J. Bone Joint Surg Am 84-A(8):1405-12). However, even with these treatments, a number of patients still must undergo corrective surgery.

As a last resort, a patient may undergo implant revision operation or strut allograft augmentation. The complication rates for these procedures is substantial, including periprosthetic fractures that fail to heal, olecranon fractures, permanent ulnar nerve injury, and triceps insufficiency. (Sanchez-Sotelo, J., 2002, J. Bone Joint Surg. Am. 84-A(9):1642-50). Thus, there remains a need for more efficacious therapies for the treatment of aseptic loosening of joint replacements and conditions associated therewith.

2.3 Chronic Otitis Media

Chronic otitis media (COM) describes a variety of signs, symptoms, and physical findings that result from the long-term damage to the middle ear by infection and inflammation. The disease is characterized by the following: perforation of the eardrum, scarring or erosion of the small, sound conducting bones of the middle ear, chronic or recurring infected drainage from the ear damage to surrounding structures such as the balance or hearing organs of the inner ear, the facial nerve, or the brain and its coverings, the meninges. Common infectious agents leading to chronic otitis media are respiratory syncytial virus, influenza viruses, parainfluenza viruses, enteroviruses and adenovirus. Current therapy options for COM include antimicrobials, extracellular antioxidants (e.g., glutathione administered by nasal aerosol, see Testa, B. et al., 2001, Laryngoscope 111(8):1486-9), steroids (see Mandel, E. et al., 2002, Pediatrics 110(6):1071-80), antibiotics (e.g., antibiotics that target the listed infectious agents)(see Cripps A. and Kyd, J., 2003, Immunol. Cell Biol. 81(1):46-51), surgery (e.g., myringotomy and adenoidectomy, see Haynes, D. and Harley, D., 2002, Otolaryngol Clin. North Am. 35(4):827-39), and Co2 laser-assisted tympanic membrane fenestration, see Garin, P. et al., 2001, J. Clin. Laser Med. Surg. 19(4):185-7). Thus, there remains a need for more efficacious therapies for the treatment of chronic otitis media.

2.4 Wilson's Disease

Wilson's disease, also known as hepatolenticular degeneration, is a rare inherited systemic disorder of copper metabolism. Individuals with Wilson's disease are unable to excrete copper into their bile, thus, copper begins to accumulate in the liver. When the liver's storage capacity is exceeded, copper then begins to collect in other organs, particularly the brain, eyes, and kidneys. This may cause acute or chronic hepatitis (inflammation of the liver) or cirrhosis (severe liver disease) due to a progressive loss of liver function. The degree of liver involvement is variable and may range from mild elevations of certain liver enzymes to complete liver failure. Symptoms associated with Wilson's disease include fatigue, anorexia, weight loss, generalized weakness, ascites (fluid accumulation in the abdomen) and abdominal swelling, or jaundice. Other findings may include enlargement of the liver (hepatomegaly), spleen (splenomegaly), or both (hepatosplenomegaly).

Current methods of treating Wilson's disease include methods of removing excess copper with chelating agents. The most common agent used for this purpose is D-penicillamine (Cuprimine®, Depen®). This drug binds to copper and forms a stable compound that is then released in urine. D-penicillamine depletes pyridoxine or Vitamin B6 from the body. Therefore, dietary supplementation with pyridoxine is required. The side effects of D-penicillamine range from minor disturbances to severe or life-threatening complications, such as aplastic anemia, immune complex nephritis, systemic lupus erythematosus, or myasthenia gravis. In some individuals, neurologic symptoms may worsen during penicillamine therapy. Thus, there is a need in the art for improved methods of treating Wilson's disease, and the symptoms associated therewith.

2.5 Gorham-Stout Disease

Gorham-Stout disease (GSD), or massive osteolysis, is an extremely rare osteolytic condition characterized by acute spontaneous resorption of bone, without any sign of malignant or infectious disorder. The lesion may develop in any part of the skeleton, with extensive bone loss, but is benign. Given the rare nature of the disease, little is known about its etiology, or possible treatment options. Current methods of treating GSD include radiotherapeutic treatment, (see Handl-Zeller, L. and Hohenberg, G., 1990, Br. J. Radiol. 63(747):206-8), use of neutralizing antibodies to IL-6 (see Devlin, R. et al., 1996, J. Clin. Endocrinol. Metab. 81(5): 1893-7), and bone grafting and irradiation (see Giraudet-Le, Q. et al., 1995, Presse Med. 24(15):719-21). Thus, there remains a need for more efficacious therapies for treatment of Gorham-Stout disease.

2.6 Hypertrophic Pulmonary Osteoarthropathy

Hypertrophic pulmonary osteoarthropathy (HPOA) is a secondary disease associated with one of various primary diseases, that is generally characterized by pernosteal new bone on the long bones, usually in the wrists and knees. Symptoms may include pain, subperiosteal new bone formation at the distal ends of long bones, metatarsals, metacarpals and proximal phalanges symmetrical arthropathy of adjacent joints clubbing of the fingers gynaecomastia. Ninety percent of HPOA cases are linked to bronchogenic carcinoma, especially peripheral squamous cell tumours, other intrathoracic tumors, chronic lung sepsis and chronic liver disease. See Mito, K. et al., 2001, Intern Med. 40(6): 532-50 (non-small cell lung cancer), Kishi, K., et al., 2002, Lung Cancer 38(3):317-20 (lung cancer), Garske, L. and Bell, S., 2002, Chest 121(4):1363-4 (lung cancer associated with cystic fibrosis). Current treatment options for HPOA include any treatment available for the primary disease, such as cancer treatments or treatments for the symptoms of cystic fibrosis. Additional non-limiting treatment options include inhibitors of osteoclastic bone resorption (e.g., IV administration of pamidronate, see Garske, L. and Bell, S., 2002, Chest 121(4):1363-4).

### 3. SUMMARY OF THE INVENTION

The present invention provides protocols for the prevention, treatment management, and/or amelioration of periodontal disease, Gorham-Stout disease, Wilson's disease, chronic otitis media, hypertrophic pulmonary osteoarthropathy, and/or aseptic loosening of joint replacement or a symptom or condition associated therewith. In particular, the present invention encompasses treatment protocols that provide better prophylactic and therapeutic profiles than current single agent therapies or combination therapies for periodontal disease, Gorham-Stout disease, Wilson's disease, chronic otitis media, hypertrophic pulmonary osteoarthropathy and/or aseptic loosening of joint replacement or symptoms or conditions associated therewith. The invention encompasses methods of administering integrin αᵥβ₃ antagonists alone or in combination with at least one other therapy such that efficacy is improved while safety is not compromised. The invention provides methods for preventing, treating, managing, or ameliorating periodontal disease, Gorham-Stout disease, Wilson's disease, chronic otitis media, hypertrophic pulmonary osteoarthropathy, or aseptic loosening of joint replacement, or a symptom or condition associated therewith, said methods comprising administering to a subject in need thereof an integrin αᵥβ₃ antagonist alone or in combination with at least one other therapy. In one embodiment, the integrin αᵥβ₃ antagonist is an antibody or antibody fragment that immunospecifically binds to integrin αᵥβ₃. In accordance with this embodiment, the antibody or antibody fragment may be conjugated or fused to a therapeutic moiety or drug. In a preferred embodiment, the integrin αᵥβ₃ antagonist is VITAXIN™ or an antigen-binding fragment thereof. In another embodiment, the integrin αᵥβ₃ antagonist is VITAXIN™ or an antigen-binding fragment thereof conjugated or fused to a therapeutic moiety or drug. In another embodiment, the integrin αᵥβ₃ antagonist is not an antibody that immunospecifically binds to integrin αᵥβ₃. In yet another embodiment, the integrin αᵥβ₃ is not VITAXIN™ or an antigen-binding fragment thereof.

The invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or a symptom or condition associated therewith, said methods comprising administering to a subject in need thereof a dose of a prophylactically or therapeutically effective amount of an integrin αᵥβ₃ antagonist (preferably, an antibody or antibody fragment that immunospecifically binds integrin αᵥβ₃, most preferably VITAXIN™ or an antigen-binding fragment thereof). The present invention also provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or a symptom or condition associated therewith, said method comprising: (a) administering to a subject in need thereof as least one dose of a prophylactically or therapeutically effective amount of an integrin αᵥβ₃ antagonist (preferably, an antibody or antibody fragment that immunospecifically binds integrin αᵥβ₃, most preferably VITAXIN™ or an antigen-binding fragment thereof); and (b) monitoring the plasma level/concentration of the integrin αᵥβ₃ antagonist in said subject after administration of a certain number of doses of the integrin αᵥβ₃ antagonist (e.g., 1, 2, 3, 4, 5, 6, 7, 8, 9, 10 or 12 doses). Preferably, the integrin αᵥβ₃ antagonist used is VITAXIN™ or an antigen-binding fragment thereof.

The present invention provides methods of administering the prophylactic or therapeutic agents of the invention locally to the area in need of treatment, for example, by local infusion, by injection, or by means of an implant, said implant being of a porous or non-porous, or gelatinous material, including membranes and matrices, such as sialastic membranes, or fibers. In a specific embodiment, the method of the invention comprises administering to a subject in need thereof a local dose of a prophylactically or therapeutically effective amount of an integrin αᵥβ₃ antagonist (preferably an antibody or antibody fragment that immunospecifically binds integrin αᵥβ₃, most preferably VITAXIN™ or an antigen-binding fragment thereof) and optionally, a dose of a prophylactically or therapeutically effective amount of at least one other therapy (e.g., a prophylactic or therapeutic agent other than an integrin αᵥβ₃ antagonist).

The invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or a symptom or condition associated therewith, said methods comprising administering to a subject in need thereof a prophylactically or therapeutically effective amount of a prophylactic or therapeutic agent (in particular, an integrin αᵥβ₃ antagonist) using ex vivo and/or in vivo gene therapy. In a specific embodiment, the invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or a symptom or condition associated therewith, said methods comprising the steps of contacting cells (preferably, cells that are autologous to the subject) with a nucleotide sequence comprising a nucleic acid sequence encoding an integrin αᵥβ₃ antagonist (preferably, an antibody or antibody fragment that immunospecifically binds integrin αᵥβ₃, and most preferably VITAXIN™ or an antigen-binding fragment thereof), and administering the cells to a subject in need thereof. In another embodiment, the invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or a symptom or condition associated therewith, said methods comprising the steps of transfecting cells (preferably, cells that are autologous to the subject) with a nucleotide sequence comprising a nucleic acid sequence encoding an integrin αᵥβ₃ antagonist (preferably an antibody or antibody fragment that immunospecifically binds integrin αᵥβ₃, and most preferably VITAXIN™ or an antigen-binding fragment thereof) and administering the transfected cells to a subject in need thereof.

In another embodiment, the invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or a symptom or condition associated therewith, said methods comprising: (i) contacting cells (preferably, cells that are autologous to the subject) with a first nucleotide sequence comprising a nucleic acid sequence encoding an integrin αᵥβ₃ antagonist (preferably an antibody or antibody fragment that immunospecifically binds integrin αᵥβ₃, and most preferably VITAXIN™ or an antigen-binding fragment thereof) prior to, subsequent to, or concurrently while contacting the cells with a second nucleotide sequence comprising nucleic acid sequence encoding a prophylactic or therapeutic agent other than an integrin αᵥβ₃ antagonist; and (ii) administering the cells to a subject in need thereof. In another embodiment, the invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or a symptom or condition associated therewith, said method comprising: (i) transfecting cells (preferably, cells that are autologous to the subject) with a first nucleotide sequence comprising a nucleic acid sequence encoding an integrin αᵥβ₃ antagonist (preferably an antibody or antibody fragment that immunospecifically binds integrin αᵥβ₃, and most preferably VITAXIN™ or an antigen-binding fragment thereof) prior to, subsequent to, or concurrently while transfecting the cells with a second nucleotide sequence comprising nucleic acid sequence encoding a prophylactic or therapeutic agent other than an integrin αᵥβ₃ antagonist; and (ii) administering the transfected cells to a subject in need thereof.

In another embodiment, the invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or a symptom or condition associated therewith, said methods comprising administering to a subject in need thereof a nucleotide sequence comprising a nucleic acid sequence encoding an integrin αᵥβ₃ antagonist (preferably an antibody or antibody fragment that immunospecifically binds integrin αᵥβ₃, and most preferably VITAXIN™ or an antigen-binding fragment thereof). In another embodiment, the invention provides methods of preventing, treating, managing or ameliorating periodontal disease, Gorham-Stout disease, Wilson's disease, chronic otitis media, hypertrophic pulmonary osteoarthropathy or aseptic loosening of joint replacement (prostheses), or a symptom or condition associated therewith, said methods comprising administering to a subject in need thereof a nucleotide sequence comprising a nucleic acid sequence encoding an integrin αᵥβ₃ antagonist (preferably an antibody or antibody fragment that immunospecifically binds integrin αᵥβ₃, and most preferably VITAXIN™ or an antigen-binding fragment thereof) prior to, subsequent to, or concurrently while administering to the subject a second nucleotide sequence comprising nucleic acid sequence encoding a prophylactic or therapeutic agent other than an integrin αᵥβ₃ antagonist.

In accordance with the foregoing embodiments, the nucleotide sequence(s) may be administered using any technique well-known in the art, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus.

The invention provides combination therapies for prevention, treatment, management, or amelioration of periodontal disease, Gorham-Stout disease, Wilson's disease, chronic otitis media, hypertrophic pulmonary osteoarthropathy or aseptic loosening of joint replacement, or a symptom or condition associated therewith, said combination therapies comprising administering to a subject in need thereof a dose of a prophylactically or therapeutically effective amount of an integrin αᵥβ₃ antagonist (preferably an antibody or antibody fragment that immunospecifically binds integrin αᵥβ₃, most preferably VITAXIN™ or an antigen-binding fragment thereof) and a dose of a prophylactically or therapeutically effective amount of at least one other therapy (e.g., a prophylactic or therapeutic agent other than an integrin αᵥβ₃ antagonist). In one embodiment, the invention provides combination therapies for the prevention, treatment, management, or amelioration of periodontal disease, Gorham-Stout disease, Wilson's disease, chronic otitis media, hypertrophic pulmonary osteoarthropathy or aseptic loosening of joint replacement, or a symptom or condition associated therewith, said combination therapies comprising administering to a subject in need thereof a dose of a prophylactically or therapeutically effective amount of an integrin αᵥβ₃ antagonist (preferably an antibody or antibody fragment that immunospecifically binds integrin αᵥβ₃, most preferably VITAXTIN™ or an antigen-binding fragment thereof) and a dose of a prophylactically or therapeutically effective amount of at least one other therapy (e.g., a prophylactic or therapeutic agent) which has a different mechanism of action than the integrin αᵥβ₃ antagonist. The invention also provides methods of preventing, treating, managing or ameliorating periodontal disease, Gorham-Stout disease, Wilson's disease, chronic otitis media, hypertrophic pulmonary osteoarthropathy or aseptic loosening of joint replacement, or a symptom or condition associated therewith, said methods comprising administering to a subject in need thereof a dose of a prophylactically or therapeutically effective amount of an integrin αᵥβ₃ antagonist (preferably, an antibody or antibody fragment that immunospecifically binds integrin αᵥβ₃ most preferably VITAXIN™ or an antigen-binding fragment thereof), a dose of a prophylactically or therapeutically effective amount of a bone metabolism regulating agent (e.g., a bisphosphonate such as zoledronate), and a dose of a prophylactically or therapeutically effective amount of at least one other therapy (e.g., a prophylactic or therapeutic agent) other than an integrin αᵥβ₃ antagonist or a bone regulating agent.

The present invention provides methods for preventing, treating, managing or ameliorating periodontal disease, Gorham-Stout disease, Wilson's disease, chronic otitis media, hypertrophic pulmonary osteoarthropathy or aseptic loosening of joint replacement, or a symptom or condition associated therewith, said methods comprising administering to a subject in need thereof a dose of a prophylactically or therapeutically effective amount of an integrin αᵥβ₃ antagonist (preferably an antibody or antibody fragment that immunospecifically binds integrin αᵥβ₃, most preferably VITAXIN™ or an antigen-binding fragment thereof) and a dose of a prophylactically or therapeutically effective amount of any of the following prophylactic or therapeutic agents: anti-inflammatory agents (e.g., steriodal and NSAIDS), analgesic agents (e.g., non-steriodal anti-inflammatory agents (NSAIDS), salicylates, narcotics, and non-narcotic and anxiolytic combinations), zinc, chelating agents, anti-arthritic agents (e.g., analgesics, NSAIDS, antirheumatic agents, glucocorticoids, skin and mucous membrane agents), bone metabolism regulating agents (e.g., bisphosphonates, vitamin D compounds, and hormones), immunomodulatory agents, antibiotics, hormone replacement therapies and/or agents that inhibit metalloproteinases, agents that inhibit prostaglandin production, agents that inhibit secretory prospholipase A₂, agents that inhibit lysine decarboxylase, and dental preparations (e.g., PERIOSTAT™ tablets, and rinses).

The invention provides methods of preventing, treating, managing, or ameliorating aseptic joint (e.g., hip) loosening, or a symptom or condition associated therewith, said methods comprising administering to a subject in need thereof a dose of a prophylactically or therapeutically effective amount of an integrin αᵥβ₃ antagonist (preferably, an antibody or antibody fragment that immunospecifically binds to integrin αᵥβ₃, most preferably VITAXIN™ or an antigen-binding fragment thereof) and a dose of a prophylactically or therapeutically effective amount of any of the following prophylactic or therapeutic agents: immunomodulatory agents; antibiotics; anti-inflammatory agents; hormone replacement therapy; agents that inhibit metalloproteinases; anti-arthritic agents; analgesic agents; and/or bone metabolism regulating agents.

The invention also provides methods of preventing, treating, managing, or ameliorating periodontal disease, or a symptom or condition associated therewith, said methods comprising administering to a subject in need thereof a dose of a prophylactically or therapeutically effective amount of an integrin αᵥβ₃ antagonist (preferably, an antibody or antibody fragment that immunospecifically binds to integrin αᵥβ₃, most preferably VITAXIN™ or an antigen-binding fragment thereof) and a dose of a prophylactically or therapeutically effective amount of any of the following prophylactic or therapeutic agents: immunomodulatory agents; antibiotics; anti-inflammatory agents; dental preparations (e.g., PERIOSTAT™); anti-arthritic agents; and/or bone metabolism regulating agents.

The invention provides methods of preventing, treating, managing, or ameliorating Gorham-Stout disease or a symptom or condition associated therewith, said methods comprising administering to a subject in need thereof a dose of a prophylactically or therapeutically effective amount of an integrin αᵥβ₃ antagonist (preferably, an antibody or antibody fragment that immunospecifically binds to integrin αᵥβ₃, most preferably VITAXIN™ or an antigen-binding fragment thereof) and a dose of a prophylactically or therapeutically effective amount of any of the following prophylactic or therapeutic agents: immunomodulatory agents (e.g., anti-IL-6 antibodies); anti-inflammatory agents; agents that inhibit metalloproteinases; anti-arthritic agents; and/or bone metabolism regulating agents.

The invention also provides methods of preventing, treating, managing, or ameliorating Wilson's disease or a symptom or condition associated therewith, said methods comprising administering to a subject in need thereof a doses of a prophylactically or therapeutically effective amount of an integrin αᵥβ₃ antagonist (preferably, an antibody or antibody fragment that immunospecifically binds to integrin αᵥβ₃, most preferably VITAXIN™ or an antigen-binding fragment thereof) and a dose of a prophylactically or therapeutically effective amount of any of the following prophylactic or therapeutic agents: immunomodulatory agents; chelating agents, vitamins; zinc acetate (Galzin®); anti-inflammatory agents; anti-arthritic agents; and/or bone metabolism regulating agents.

The invention provides methods of preventing, treating, managing, or ameliorating chronic otitis media or a symptom or condition associated therewith, said methods comprising administering to a subject in need thereof a dose of a prophylactically or therapeutically effective amount of an integrin αᵥβ₃ antagonist (preferably, an antibody or antibody fragment that immunospecifically binds to integrin αᵥβ₃, most preferably VITAXIN™ or an antigen-binding fragment thereof) and a dose of a prophylactically or therapeutically effective amount of at least one of any of the following prophylactic or therapeutic agents: immunomodulatory agents; anti-inflammatory agents; bone metabolism regulating agents; antioxidants (e.g., glutathione administered by nasal aerosol, see Testa, B. et al., 2001, Laryngoscope 111(8):1486-9), antivirals and/or antibiotics.

The invention also provides methods of preventing, treating, managing, or ameliorating hypertrophic pulmonary osteoarthropathy or a symptom or condition associated therewith, said methods comprising administering to a subject in need thereof a dose of a prophylactically or therapeutically effective amount of an integrin αᵥβ₃ antagonist (preferably, an antibody or antibody fragment that immunospecifically binds to integrin αᵥβ₃, most preferably VITAXIN™ or an antigen-binding fragment thereof) and a dose of a prophylactically or therapeutically effective amount of one or more of any of the following prophylactic or therapeutic agents: immunomodulatory agents (e.g., chemotherapy); anti-inflammatory agents; bone metabolism regulating agents, anti-arthritic agents, and/or antibiotics.

In a specific embodiment, the present invention provides a method for treating, preventing, managing or ameliorating inflammation associated with aseptic loosening of joint replacement, or a symptom thereof, said method comprising administering to a subject in need thereof a dose of a prophylactically or therapeutically effective amount of an integrin αᵥβ₃ antagonist and a dose of a prophylactially or therapeutically effective amount of one or more of the following prophylactic or therapeutic agents: anti-inflammatory agents; anti-arthritic agents; immunomodulatory agents; and/or antibiotics. In another specific embodiment, the present invention provides a method for preventing, treating, managing or ameliorating bone resorption (e.g., osteoclast resorption) associated with aseptic loosening of joint replacement, or a symptom thereof, said method comprising administering to a subject in need thereof a dose of a prophylactically or therapeutically effective amount of an integrin αᵥβ₃ antagonist and a dose of a prophylactially or therapeutically effective amount of one or more of the following prophylactic or therapeutic agents: immunomodulatory agents; anti-arthritic agents; anti-inflammatory agents; bone metabolism regulating agents; and/or agents that inhibit metalloproteinases.

In a specific embodiment, the present invention provides a method for preventing, treating, managing or ameliorating inflammation associated with periodontal disease or a symptom thereof, said method comprising administering to a subject in need thereof a dose of a prophylactically or therapeutically effective amount of an integrin αᵥβ₃ antagonist and a dose of a prophylactially or therapeutically effective amount of one or more of the following prophylactic or therapeutic agents: anti-inflammatory agents; immunomodulatory agents; anti-arthritic agents; and/or antibiotics (e.g., PERIOSTAT™). In another specific embodiment, the present invention provides a method for preventing, treating, managing or ameliorating bone resorption (in particular, osteoclast resorption) associated with periodontal disease or a symptom thereof, said method comprising administering to a subject in need thereof a dose of a prophylactically or therapeutically effective amount of an integrin αᵥβ₃ antagonist and a dose of a prophylactically or therapeutically effective amount of one or more prophylactic or therapeutic agents: immunomodulatory agents, anti-inflammatory agents; bone metabolism regulating agents and/or agents that inhibit metalloproteinases.

In a specific embodiment, the present invention provides a method for preventing, treating, managing or ameliorating inflammation associated with Gorham-Stout disease or a symptom thereof, said method comprising administering to a subject in need thereof a dose of a prophylactically or therapeutically effective amount of an integrin αᵥβ₃ antagonist and a dose of a prophylactially or therapeutically effective amount of one or more of the following prophylactic or therapeutic agents: anti-inflammatory agents; immunomodulatory agents (e.g., anti-IL-6 antibodies); and/or anti-arthritic agents. In another specific embodiment, the present invention provides a method for preventing, treating, managing or ameliorating bone resorption (in particular, osteoclast resorption) associated with Gorham-Stout disease or a symptom thereof, said method comprising administering to a subject in need thereof a dose of a prophylactically or therapeutically effective amount of an integrin αᵥβ₃ antagonist and a dose of a prophylactically or therapeutically effective amount of one or more of the following prophylactic or therapeutic agents: immunomodulatory agents (e.g., anti-IL-6 antibodies); anti-inflammatory agents; bone metabolism regulating agents and/or agents that inhibit metalloproteinases.

In a specific embodiment, the present invention provides a method for preventing, treating, managing or ameliorating inflammation associated with hypertrophic pulmonary osteoarthropathy or a symptom thereof, said method comprising administering to a subject in need thereof a dose of a prophylactically or therapeutically effective amount of an integrin αᵥβ₃ antagonist and a dose of a prophylactially or therapeutically effective amount of one or more of the following prophylactic or therapeutic agents: anti-inflammatory agents; immunomodulatory agents (e.g., chemotherapeutic agents); and/or anti-arthritic agents. In another specific embodiment, the present invention provides a method for preventing, treating, managing or ameliorating bone resorption (in particular, osteoclast resorption) associated with hypertrophic pulmonary osteoarthropathy or a symptom thereof, said method comprising administering to a subject in need thereof a dose of a prophylactically or therapeutically effective amount of an integrin αᵥβ₃ antagonist and a dose of a prophylactically or therapeutically effective amount of one or more of the following prophylactic or therapeutic agents: immunomodulatory agents (e.g., chemotherapeutic agents); anti-inflammatory agents; bone metabolism regulating agents and/or agents that inhibit metalloproteinases.

In a specific embodiment, the present invention provides a method for preventing, treating, managing or ameliorating inflammation associated with Wilson's disease or a symptom thereof, said method comprising administering to a subject in need thereof a dose of a prophylactically or therapeutically effective amount of an integrin αᵥβ₃ antagonist and a dose of a prophylactially or therapeutically effective amount of one or more of the following prophylactic or therapeutic agents: anti-inflammatory agents; immunomodulatory agents; and/or anti-arthritic agents. In another specific embodiment, the present invention provides a method for preventing, treating, managing or ameliorating inflammation associated with chronic otitis media or a symptom thereof, said method comprising administering to a subject in need thereof a dose of a prophylactically or therapeutically effective amount of an integrin αᵥβ₃ antagonist and a dose of a prophylactially or therapeutically effective amount of one or more of the following prophylactic or therapeutic agents: anti-inflammatory agents; immunomodulatory agents; and/or antibiotics.

The present invention provides methods of preventing, treating, managing or ameliorating periodontal disease, Gorham-Stout disease, Wilson's disease, chronic otitis media, hypertrophic pulmonary osteoarthropathy or aseptic loosening of joint replacement, or a symptom or condition associated therewith, said methods comprising administering to a subject in need thereof a dose of a prophylactically or therapeutically effective amount of an integrin αᵥβ₃ antagonist (preferably, an antibody or antibody fragment that immunospecifically binds integrin αᵥβ₃, most preferably VITAXIN™ or an antigen-binding fragment thereof) and a prophylactically and therapeutically effective amount of at least one therapy other than an integrin αᵥβ₃ antagonist (e.g., a prophylactic or therapeutic agent such as an analgesic, anti-arthritic, bone metabolism regulating agent, antibiotic, anti-imflammatory agent and immunomodulatory agent), wherein the dose of the effective amount of said an integrin αᵥβ₃ antagonist is administered once every 3 days, preferably once every 4 days, once every 5 days, once every 6 days, once every week, once every two weeks, once every three weeks or once a month. The present invention also provides methods of preventing, treating, managing, or ameliorating periodontal disease, Gorham-Stout disease, Wilson's disease, chronic otitis media, hypertrophic pulmonary osteoarthropathy, or aseptic loosening of joint replacement (prostheses), or a symptom or condition associated therewith, said method comprising: (a) administering to a subject in need thereof a dose of a prophylactically or therapeutically effective amount of an integrin αᵥβ₃ antagonist (preferably, an antibody or antibody fragment that immunospecifically binds integrin αᵥβ₃, most preferably VITAXIN™ or an antigen-binding fragment thereof) and a dose of a prophylactically or therapeutically effective amount of at least one therapy (e.g., a prophylactic or therapeutic agent) other than an integrin αᵥβ₃ antagonist; and (b) monitoring the plasma level/concentration of the integrin αᵥβ₃ antagonist in said subject after the administration of a certain number of doses of the integrin αᵥβ₃ antagonist (e.g., 1, 2, 3, 4, 5, 6, 7, 8, 9, 10, 11 or 12 doses).

The present invention provides methods of preventing, treating, managing or ameliorating periodontal disease or a symptom thereof, said methods comprising administering to a subject in need thereof a dose of a prophylactically or therapeutically effective amount of an integrin αᵥβ₃ antagonist (preferably, an antibody or antibody fragment that immunospecifically binds integrin αᵥβ₃, most preferably VITAXIN™ or an antigen-binding fragment thereof) in combination with or as an adjunct to at least one of the following therapies for periodontal disease: CO₂ laser therapy; scaling; root planing; periodontal surgery. In a specific embodiment, the methods further comprise administering to the subject a dose of a prophylactically and therapeutically effective amount of at least one therapy other than an integrin αᵥβ₃ antagonist (e.g., a prophylactic or therapeutic agent such as an analgesic, anti-arthritic, bone metabolism regulating agent, anti-inflammatory agent and dental preparation).

The present invention provides methods of preventing, treating, managing or ameliorating aseptic loosening of joint replacement or a symptom or condition associated therewith, said methods comprising administering to a subject in need thereof a dose of a prophylactically or therapeutically effective amount of an integrin αᵥβ₃ antagonist (preferably, an antibody or antibody fragment that immunospecifically binds integrin αᵥβ₃, most preferably VITAXIN™ or an antigen-binding fragment thereof) in combination with surgery. In a specific embodiment, the methods further comprise administering to the subject a dose of a prophylactically and therapeutically effective amount of at least one therapy other than an integrin αᵥβ₃ antagonist (e.g., a prophylactic or therapeutic agent such as an analgesic, anti-arthritic, bone metabolism regulating agent, and anti-inflammatory agent).

The present invention provides methods of preventing, treating, managing or ameliorating hypertrophic pulmonary osteoarthropathy or a symptom or condition associated therewith, said methods comprising administering to a subject in need thereof a dose of a prophylactically or therapeutically effective amount of an integrin αᵥβ₃ antagonist (preferably, an antibody or antibody fragment that immunospecifically binds integrin αᵥβ₃, most preferably VITAXIN™ or an antigen-binding fragment thereof) in combination with surgery. In a specific embodiment, the methods further comprise administering to the subject a dose of a prophylactically and therapeutically effective amount of at least one therapy other than an integrin αᵥβ₃ antagonist (e.g., a prophylactic or therapeutic agent such as an analgesic, anti-arthritic, bone metabolism regulating agent, and anti-inflammatory agent).

The combination of an integrin αᵥβ₃ antagonist and at least one other therapy (e.g., at least one prophylactic or therapeutic agent other than an integrin αᵥβ₃ antagonist) produces a better prophylactic or therapeutic effect in a subject than either therapy alone. In certain embodiments, the combination of an integrin αᵥβ₃ antagonist and a therapy (e.g., a prophylactic or therapeutic agent) other than an integrin αᵥβ₃ antagonist achieves a 2 fold, preferably a 3 fold, 4 fold, 5 fold, 6 fold, 7 fold, 8 fold, 9 fold, 10 fold, 15 fold or 20 fold better prophylactic or therapeutic effect in a subject with periodontal disease, Gorham-Stout disease, Wilson's disease, chronic otitis media, hypertrophic pulmonary osteoarthropathy or aseptic loosening of joint replacement or a condition associated therewith, than either treatment alone. In other embodiments, the combination of an integrin αᵥβ₃ antagonist and a therapy (e.g., a prophylactic or therapeutic agent) other than an integrin αᵥβ₃ antagonist achieves a 10%, preferably 15%, 20%, 25%, 30%, 35%, 40%, 45%, 50%, 55%, 60%, 65%, 70%, 75%, 80%, 85%, 90%, 95%, 100%, 125%, 150%, or 200% better prophylactic or therapeutic effect in a subject with periodontal disease, Gorham-Stout disease, Wilson's disease, chronic otitis media, hypertrophic pulmonary osteoarthropathy or aseptic loosening of joint replacement or a condition associated therewith, than either therapy alone. In other embodiments, the combination of an integrin αᵥβ₃ antagonist and at least one therapy other than integrin αᵥβ₃ antagonist (e.g., a prophylactic or therapeutic agent) has more than an additive effect or synergistic effect in a subject with periodontal disease, Gorham-Stout disease, Wilson's disease, chronic otitis media, hypertrophic pulmonary osteoarthropathy or aseptic loosening of joint replacement or a condition associated therewith.

The combination therapies of the invention enable lower dosages of therapies (e.g., prophylactic or therapeutic agents) to be utilized in conjugation with integrin αᵥβ₃ antagonists for the prevention, treatment, management or amelioration of periodontal disease, Gorham-Stout disease, Wilson's disease, chronic otitis media, hypertrophic pulmonary osteoarthropathy or aseptic loosening of joint replacement or a condition associated therewith, and/or less frequent administration of such therapies to a subject with periodontal disease, Gorham-Stout disease, Wilson's disease, chronic otitis media, hypertrophic pulmonary osteoarthropathy, or aseptic loosening of joint replacement or a condition associated therewith, to achieve a prophylactic or therapeutic effect. The combination therapies of the invention reduce or avoid unwanted or adverse side effects associated with the administration of current single agent therapies and/or existing combination therapies for periodontal disease, Gorham-Stout disease, Wilson's disease, chronic otitis media, hypertrophic pulmonary osteoarthropathy or aseptic loosening of joint replacement or a condition associated therewith, which in turn improves patient compliance with the treatment protocol. Further, the combination therapies of the invention reduce the dosages of an integrin αᵥβ₃ antagonist and/or frequency of administration of dosages of an integrin αᵥβ₃ antagonist to a subject with periodontal disease, Gorham-Stout disease, Wilson's disease, chronic otitis media, hypertrophic pulmonary osteoarthropathy or aseptic loosening of joint replacement or a condition associated therewith, to improve the quality of life of said subject.

The therapies (e.g., prophylactic or therapeutic agents) of the combination therapies of the present invention can be administered concurrently or sequentially to a subject. The therapies (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) prophylactic or therapeutic agent for a period of time and repeating this sequential administration, i.e., the cycle, in order to reduce the development of resistance to one of the agents, to avoid or reduce the side effects of one of the agents, and/or to improve the efficacy of the therapy.

The therapies (e.g., prophylactic or therapeutic agents) of the combination therapies of the invention can be administered to a subject concurrently. The term “concurrently” is not limited to the administration of prophylactic or therapeutic agents at exactly the same time, but rather it is meant that an integrin αᵥβ₃ antagonist and the other agent are administered to a subject in a sequence and within a time interval such that the integrin αᵥβ₃ antagonist can act together with the other therapy (e.g., agent) to provide an increased benefit than if they were administered otherwise. For example, each therapy (e.g., prophylactic or therapeutic agent) may be administered at the same time or sequentially in any order at different points in time; however, if not administered at the same time, they should be administered sufficiently close in time so as to provide the desired therapeutic or prophylactic effect. Each therapy (e.g., prophylactic or therapeutic agent) can be administered separately, in any appropriate form and by any suitable route. In various embodiments, the therapies (e.g., prophylactic or therapeutic agents) are administered less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In preferred embodiments, two or more therapies (e.g., prophylactic or therapeutic agents) are administered within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

The present invention provides pharmaceutical compositions comprising an integrin αᵥβ₃ antagonist and a pharmaceutically acceptable carrier. The present invention encompasses the use of pharmaceutical compositions comprising a prophylactic or therapeutic agent other than an integrin αᵥβ₃ antagonist. The present invention provides pharmaceutical compositions comprising an integrin αᵥβ₃ antagonist and at least one therapy other than an integrin αᵥβ₃ antagonist (e.g., a prophylactic or therapeutic agent such as an analgesic, anti-arthritic, bone metabolism regulating agent, anti-inflammatory agent, or dental preparation). The present invention also provides pharmaceutical compositions comprising an integrin αᵥβ₃ antagonist, at least one bone metabolism regulating agents (e.g., a bisphosphonate such as zoledronate), at least one prophylactic or therapeutic agent other than an integrin αᵥβ₃ antagonist, and a pharmaceutically acceptable carrier.

In one embodiment, the pharmaceutical composition comprises a nucleotide sequence comprising a nucleic acid sequence encoding an integrin αᵥβ₃ antagonist (preferably, an antibody or antibody fragment that immunospecifically binds to integrin αᵥβ₃, most preferably VITAXIN™ or an anitgen-binding fragment thereof). In another embodiment, the pharmaceutical composition comprises a nucleotide sequence comprising a nucleic acid sequence encoding an integrin αᵥβ₃ antagonist (preferably an antibody or antibody fragment that immunospecifically binds integrin αᵥβ₃, most preferably VITAXIN™ or an antigen-binding fragment thereof), and a nucleotide sequence comprising a nucleic acid sequence encoding a prophylactic or therapeutic agent other than an integrin αᵥβ₃ antagonist.

In a specific embodiment, a pharmaceutical composition comprises an integrin αᵥβ₃ antagonist (preferably, an antibody or antibody fragment that immunospecifically binds to integrin αᵥβ₃, most preferably VITAXIN™ or an anitgen-binding fragment thereof), at least one analgesic, and a pharmaceutically acceptable carrier, wherein the analgesic is not an integrin αᵥβ₃ antagonist. In another embodiment, a pharmaceutical composition comprises an integrin αᵥβ₃ antagonist (preferably, an antibody or antibody fragment that immunospecifically binds to integrin αᵥβ₃, most preferably VITAXIN™ or an anitgen-binding fragment thereof), at least one anti-arthritic agent, and a pharmaceutically acceptable carrier, wherein the anti-arthritic agent is not an integrin αᵥβ₃ antagonist. In another embodiment, a pharmaceutical composition comprises an integrin αᵥβ₃ antagonist (preferably, an antibody or antibody fragment that immunospecifically binds to integrin αᵥβ₃, most preferably VITAXIN™ or an anitgen-binding fragment thereof), at least one bone metabolism regulating agent, and a pharmaceutically acceptable carrier, wherein the bone metabolism regulating agents is not an integrin αᵥβ₃ antagonist.

In a specific embodiment, a pharmaceutical composition comprises an integrin αᵥβ₃ antagonist (preferably, an antibody or antibody fragment that immunospecifically binds to integrin αᵥβ₃, most preferably VITAXIN™ or an anitgen-binding fragment thereof), at least one anti-inflammatory agent, and a pharmaceutically acceptable carrier, wherein the anti-inflammatory agent is not an integrin αᵥβ₃ antagonist. In another embodiment, a pharmaceutical composition comprises an integrin αᵥβ₃ antagonist (preferably, an antibody or antibody fragment that immunospecifically binds to integrin αᵥβ₃, most preferably VITAXIN™ or an anitgen-binding fragment thereof), at least one dental preparation, and a pharmaceutically acceptable carrier, wherein the dental preparation is not an integrin αᵥβ₃ antagonist. In another embodiment, a pharmaceutical composition comprises an integrin αᵥβ₃ antagonist (preferably, an antibody or antibody fragment that immunospecifically binds to integrin αᵥβ₃, most preferably VITAXIN™ or an anitgen-binding fragment thereof), at least one antibiotic, and a pharmaceutically acceptable carrier. In another embodiment, a pharmaceutical composition comprises an integrin αᵥβ₃ antagonist (preferably, an antibody or antibody fragment that immunospecifically binds to integrin αᵥβ₃, most preferably VITAXIN™ or an anitgen-binding fragment thereof), at least one immunomodulatory agent (e.g., a chemotherapeutic agent or an immunomodulatory agent other than a chemotherapeutic agent), and a pharmaceutically acceptable carrier.

The invention encompasses sustained release formulations for the administration of an integrin αᵥβ₃ antagonist (preferably, an antibody or antibody fragment that immunospecifically binds integrin αᵥβ₃, most preferably VITAXIN™ or an anitgen-binding fragment thereof) and/or at least one prophylactic or therapeutic agent other than an integrin αᵥβ₃ antagonist to a subject. The sustained release formulations reduce the dosage and/or frequency of administration of such agents to a subject.

The pharmaceutical compositions of the invention may be used in accordance with the methods of the invention for the prevention, treatment, management or amelioration of periodontal disease, Gorham-Stout disease, Wilson's disease, chronic otitis media, hypertrophic pulmonary osteoarthropathy or aseptic loosening of joint replacement or a symptom or condition associated therewith. Preferably, the pharmaceutical compositions of the invention are sterile and in suitable form for a particular method of administration to a subject with periodontal disease, Gorham-Stout disease, Wilson's disease, chronic otitis media, hypertrophic pulmonary osteoarthropathy or aseptic loosening of joint replacement or a symptom or condition associated therewith.

The compositions and methods described herein are useful for the prevention, treatment, management, or amelioration of periodontal diseases including, but not limited to, gingivitis, periodontitis, and periodontosis. The compositions and methods described herein are also useful for the prevention, treatment, management or amelioration of aseptic loosening of joint or bone replacement (e.g., hip or knee replacement) or a condition or symptom associated therewith including, but not limited to, osteolysis (especially periprosthetic osteolysis), osteoclastogenesis, osteoporosis, arthritis, and dysplasia. In particular, the compositions and methods described herein are useful for the prevention, treatment, management or amelioration of a symptom associated with inflammatory osteolysis, other disorders characterized by abnormal bone resorption, or disorders characterized by bone loss (e.g., osteoporosis).

The compositions and methods described herein are useful for the prevention, treatment, management or amelioration of chronic otitis media, or a condition or symptom associated therewith. The compositions and methods described herein are also useful for the prevention, treatment, management or amelioration of Gorham-Stout disease, Wilson's disease, or hypertrophic pulmonary osteoarthropathy (HPOA) or a condition or symptom associated therewith.

The progression, regression or stasis of periodontal disease, Gorham-Stout disease, Wilson's disease, chronic otitis media, hypertrophic pulmonary osteoarthropathy or aseptic loosening of joint replacement, or conditions associated therewith in a subject may be monitored or assessed utilizing an integrin αᵥβ₃ antagonist (preferably, an antibody or antibody fragment that immunospecifically binds to integrin αᵥβ₃) to measure or detect the expression of integrin αᵥβ₃. Other techniques for measuring, monitoring and assessing the extent of periodontal disease, Gorham-Stout disease, Wilson's disease, chronic otitis media, hypertrophic pulmonary osteoarthropathy or aseptic loosening of joint replacement, or conditions associated therewith can be used in conjunction with methods for measuring or detecting the expression of integrin αᵥβ₃ using an integrin αᵥβ₃ antagonist. Techniques for measuring, monitoring and assessing the extent of periodontal disease include, but are not limited to: measuring periodontal bone and attachment loss, especially by digital subtraction radiology, measuring the extent of gingival inflammation and bleeding, the probing depth of the pocket to the point of resistance, the clinical attachment loss of the periodontal ligament measured from a fixed point of the tooth (usually the cemento-enamel junction), the loss of adjacent alveolar bone as measured by x-ray, and plaque accumulation. Current methods known in the art of measuring the progression or regression of aseptic loosening of joint replacement, or conditions associated therewith include but are not limited to measurement of bone-remodeling, osteolysis, linear/volumetric wear, Harris hip score, the Merle d'Aubign and Postel hip score, the McMaster-Toronto Arthritis Patient Preference Disability Questionnaire (MACTAR), the Western Ontario and McMaster University Osteoarthritis Index (WOMAC), measuring bone volume in vivo by microcomputed tomography analysis and bone histomorphometry analysis.

The present invention provides article of manufactures comprising packaging material and a pharmaceutical composition of the invention in suitable form for administration to a subject contained within said packaging material. In particular, the present invention provides articles of manufacture comprising packaging material and a pharmaceutical composition in suitable form for administration to a human contained within said packaging material, wherein said pharmaceutical composition comprises an integrin αᵥβ₃ antagonist and a pharmaceutically acceptable carrier. The present invention also provides articles of manufacture comprising packaging material and two pharmaceutical compositions in suitable form for administration to a human contained within said packaging material, wherein the first pharmaceutical composition comprises an integrin αᵥβ₃ antagonist and a pharmaceutically acceptable carrier, and the second pharmaceutical composition comprises a prophylactic or therapeutic agent other than an integrin αᵥβ₃ antagonist and a pharmaceutically acceptable carrier. The present invention also provides articles of manufacture comprising packaging material and a pharmaceutical composition of the invention in suitable form for administration to a subject contained within said packaging material wherein said pharmaceutical composition comprises an integrin αᵥβ₃ antagonist (preferably, an antibody or antibody fragment that immunospecifically binds integrin αᵥβ₃, most preferably VITAXIN™ or an antigen-binding fragment thereof), at least one prophylactic or therapeutic agent other than an integrin αᵥβ₃ antagonist, and a pharmaceutically acceptable carrier. The articles of manufacture of the invention may include instructions regarding the use or administration of a pharmaceutical composition, or other informational material that advises the physician, technician or patient on how to appropriately prevent, treat, manage or ameliorate the disease or disorder in question.

3.1 Terminology

As used herein, the term “analog” in the context of proteinaceous agent (e.g., proteins, polypeptides, peptides, and antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength 3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possess a similar or identical function as a first organic or inorganic molecule and is structurally similar to the first organic or inorganic molecule.

As used herein, the terms “antagonist” and “antagonists” refer to any protein, polypeptide, peptide, peptidomimetic, glycoprotein, antibody, antibody fragment, carbohydrate, nucleic acid, organic molecule, inorganic molecule, large molecule, or small molecule that blocks, inhibits, reduces or neutralizes the function, activity and/or expression of another molecule. In various embodiments, an antagonist reduces the function, activity and/or expression of another molecule by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

As used herein, the terms “anti-Integrin αᵥβ₃ antibodies,” and “Integrin αᵥβ₃ antibodies,” refer to the antibodies described in Section 4.1.1 infra.

As used herein, the terms “antibody” and “antibodies” refer to monoclonal antibodies, multispecific antibodies, human antibodies, humanized antibodies, camelised antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, e.g., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e, by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

As used herein, the terms “disorder” and “disease” are used interchangeably to refer to a condition in a subject. In particular, the term “periodontal disease” is used interchangeably with the term “periodontal disorder”.

As used herein, the term “effective amount” refers to the amount of a therapy (e.g., a prophylactic or therapeutic agent) which is sufficient to reduce or ameliorate the progression, severity and/or duration of a disease or disorder (e.g., periodontal disease, Gorham-Stout disease, Wilson's disease, chronic otitis media, hypertrophic pulmonary osteoarthropathy or aseptic loosening of joint replacement, or a condition associated therewith) or a symptom thereof, prevent the recurrence of the disease or disorder (e.g., periodontal disease, Gorham-Stout disease, Wilson's disease, chronic otitis media, hypertrophic pulmonary osteoarthropathy or aseptic loosening of joint replacement, or a condition associated therewith), prevent the development or onset of a symptom associated with a disease or disorder (e.g., periodontal disease, Gorham-Stout disease, Wilson's disease, chronic otitis media, hypertrophic pulmonary osteoarthropathy or aseptic loosening of joint replacement, or a condition or symptom associated therewith), or enhance the prophylactic or therapeutic effect of another therapy. In the context of diagnosing or detecting a disease or disorder (e.g., periodontal disease, Gorham-Stout disease, Wilson's disease, chronic otitis media, hypertrophic pulmonary osteoarthropathy or aseptic loosening of joint replacement, or a condition associated therewith), an “effective amount” of an integrin αᵥβ₃ antagonist is an amount effective to detect the expression of integrin αᵥβ₃.

As used herein, the term “epitopes” refers to fragments of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide or protein (including an antibody). In a specific embodiment, a fragment of a polypeptide or protein retains at least one function of the polypeptide or protein. In another embodiment, a fragment of a polypeptide retains two, three or more functions of the second, different polypeptide or protein. Preferably, a fragment of an antibody retains the ability to immunospecifically bind to integrin αᵥβ₃.

As used herein, the term “fusion protein” refers to a polypeptide or protein that comprises an amino acid sequence of a first protein, polypeptide or functional fragment, analog or derivative thereof, and an amino acid sequence of a heterologous protein, or polypeptide (e.g., a second protein, polypeptide, or fragment, analog or derivative thereof different than the first protein, or polypeptide, fragment, analog or derivative thereof, or a second protein, polypeptide or fragment, analog, or derivative thereof not naturally associated with the first protein, polypeptide, or fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. In a preferred embodiment, fusion proteins retain or have improved activity relative to the activity of the original protein, polypeptide or peptide prior to being fused to a heterologous protein, polypeptide or peptide.

As used herein, the term “host cell” includes a subject cell transfected or transformed with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. Generally, stringent conditions are selected to be about 5 to 10° C. lower than the thermal melting point (Tm) for the specific sequence as a defined ionic strength pH. The T, is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (for example, 10 to 50 nucleotides) and at least about 60° C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

In one, non-limiting example stringent hybridization conditions are hybridization at 6× sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (e.g., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “immunomodulatory agent” and variations thereof including, but not limited to, immunomodulatory agents, immunomodulants or immunomodulatory drugs, refer to an agent that modulates a host's immune system. In a specific embodiment, an immunomodulatory agent is an agent that shifts one aspect of a subject's immune response. In certain embodiments, an immunomodulatory agent is an agent that inhibits or reduces a subject's immune system (i.e., an immunosuppressant agent). In certain other embodiments, an immunomodulatory agent is an agent that activates or increases a subject's immune system (i.e., an immunostimulatory agent). In accordance with the invention, an immunomodulatory agent used in the combination therapies of the invention does not include an antibody that immunospecifically binds to Integrin αᵥβ₃. Immunomodulatory agents include, but are not limited to, small molecules, peptides, polypeptides, proteins, nucleic acids (e.g., DNA and RNA nucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules.

As used herein, the term “immunospecifically binds to an antigen” and analogous terms refer to peptides, polypeptides, proteins, fusion proteins and antibodies or fragments thereof that specifically bind to an antigen or a fragment and do not specifically bind to other antigens. A peptide, polypeptide, proteins, fusion proteins or antibodies that immunospecifically binds to an antigen may bind to other peptides, polypeptides, proteins, fusion proteins or antibodies with lower affinity as determined by, e.g., immunoassays, BIAcore, or other assays known in the art. Antibodies or fragments that immunospecifically bind to an antigen may cross-reactive with related antigens. Preferably, antibodies or fragments that immunospecifically bind to an antigen do not cross-react with other antigens.

As used herein, the term “immunospecifically binds to integrin 3 and analogous terms refer to peptides, polypeptides, proteins, fusion proteins and antibodies or fragments thereof that specifically bind to integrin αᵥβ₃ or a fragment thereof and do not specifically bind to other polypeptides. A peptide, polypeptide, proteins, fusion proteins, antibodies, or fragment thereof that immunospecifically binds to integrin αᵥβ₃ may bind to other peptides, polypeptides, proteins, fusion proteins, antibodies, or fragment thereof with lower affinity as determined by, e.g., immunoassays, BIAcore, or other assays known in the art. Antibodies or fragments that immunospecifically bind to integrin αᵥβ₃ may be cross-reactive with related antigens. Preferably, antibodies or fragments that immunospecifically bind to integrin αᵥβ₃ or fragment thereof do not cross-react with other antigens. Antibodies or fragments that immunospecifically bind to integrin αᵥβ₃ can be identified, for example, by immunoassays, BIAcore, or other techniques known to those of skill in the art. An antibody or antibody fragment binds specifically to integrin αᵥβ₃ when it binds to integrin αᵥβ₃ with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity. In one embodiment, an antibody that immunospecifically binds to integrin αᵥβ₃ that is a fusion protein specifically binds to the portion of the fusion protein that is integrin αᵥβ₃.

As used herein, the term “in combination” refers to the use of more than one therapy (e.g., more than one prophylactic agent and/or therapeutic agent). The use of the term “in combination” does not restrict the order in which therapies (e.g., prophylactic and/or therapeutic agents) are administered to a subject with a disease or disorder (e.g., periodontal disease, Gorham-Stout disease, Wilson's disease, chronic otitis media, hypertrophic pulmonary osteoarthropathy or aseptic loosening of joint replacement, or a condition associated therewith). A first therapy (e.g., prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., prophylactic or therapeutic agent) to a subject with a disease or disorder (e.g., periodontal disease, Gorham-Stout disease, Wilson's disease, chronic otitis media, hypertrophic pulmonary osteoarthropathy or aseptic loosening of joint replacement, or a condition associated therewith).

As used herein, the term “integrin αᵥβ₃” refers to the heterodimer integrin αᵥβ₃, an analog, derivative or a fragment thereof, or a fusion protein comprising integrin αᵥβ₃, an analog, derivative or a fragment thereof. The integrin αᵥβ₃ may be from any species. The nucleotide and/or amino acid sequences of integrin αᵥβ₃ can be found in the literature or public databases, or the nucleotide and/or amino acid sequences can be determined using cloning and sequencing techniques known to one of skill in the art. For example, the nucleotide sequence of human integrin αᵥβ₃ can be found in the GenBank database (see, e.g., Accession No. NM_{—}002210 for αᵥ, and Accession No. L28832 for β₃). The amino acid sequence of human αᵥβ₃ can be found in the GenBank database (see, e.g., Accession No. AAA 61631 for αᵥ, and Accession No. S44360 for β₃). In a preferred embodiment, an integrin αᵥβ₃ is human integrin αᵥβ₃, an analog, derivative or a fragment thereof.

In a preferred embodiment, an integrin αᵥβ₃ is human integrin αᵥβ₃, an analog, derivative or a fragment thereof.

As used herein, the term “isolated” in the context of a proteinaceous agent (e.g., a peptide, polypeptide, fusion protein or antibody) refers to a proteinaceous agent (e.g., a peptide, polypeptide, fusion protein or antibody) which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent (e.g., a peptide, polypeptide, fusion protein or antibody) in which the proteinaceous agent (e.g., a peptide, polypeptide, fusion protein or antibody) is separated from cellular components of the cells from which it is isolated or recombinantly produced. Thus, a proteinaceous agent (e.g., a peptide, polypeptide, fusion protein or antibody) that is substantially free of cellular material includes preparations of a proteinaceous agent (e.g., a peptide, polypeptide, fusion protein or antibody) having less than about 30%, 20%, 10%, or 5% (by dry weight) of heterologous protein (also referred to as a “contaminating protein”). When the proteinaceous agent (e.g., the peptide, polypeptide, fusion protein or antibody) is recombinantly produced, it is also preferably substantially free of culture medium, e.g., culture medium represents less than about 20%, 10%, or 5% of the volume of the protein preparation. When the proteinaceous agent (e.g., the peptide, polypeptide, fusion protein or antibody) is produced by chemical synthesis, it is preferably substantially free of chemical precursors or other chemicals, e.g., it is separated from chemical precursors or other chemicals which are involved in the synthesis of the peptide, polypeptide, fusion protein or antibody. Accordingly such preparations of a proteinaceous agent (e.g., a peptide, polypeptide, fusion protein or antibody) have less than about 30%, 20%, 10%, 5% (by dry weight) of chemical precursors or compounds other than the proteinaceous agent (e.g., a peptide, polypeptide, fusion protein or antibody) of interest. In a preferred embodiment, an antibody of the invention is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment, a nucleic acid molecule encoding an antibody of the invention is isolated.

As used herein, the term “isolated” in the context of an organic or inorganic molecule (whether it be a small or large molecule), other than a proteinaceous agent or nucleic acid, refers to an organic or inorganic molecule substantially free of a different organic or inorganic molecule. Preferably, an organic or inorganic molecule is 60%, 65%, 70%, 75%, 80%, 85%, 90%, 95%, or 99% free of a second, different organic or inorganic molecule. In a preferred embodiment, an organic or inorganic molecule is isolated.

As used herein, the terms “manage”, “managing” and “management” refer to the beneficial effects that a subject derives from a therapy (e.g., prophylactic or therapeutic agent), which does not result in a cure of the disease. In certain embodiments, a subject is administered a therapy (e.g., a prophylactic or therapeutic agent) to “manage” a disease so as to prevent the progression or worsening of the disease.

As used herein, the terms “non-responsive” and refractory” describe patients treated with a currently available therapy (e.g., prophylactic or therapeutic agent) for a disease or disorder (e.g., periodontal disease, Gorham-Stout disease, Wilson's disease, chronic otitis media, hypertrophic pulmonary osteoarthropathy or aseptic loosening of joint replacement, or a condition associated therewith) which is not clinically adequate to relieve a symptom associated with the disease or disorder (e.g., periodontal disease, Gorham-Stout disease, Wilson's disease, chronic otitis media, hypertrophic pulmonary osteoarthropathy or aseptic loosening of joint replacement, or a condition associated therewith). Typically, such patients suffer from severe, persistently active disease and require additional therapy to ameliorate the symptoms associated with their disease or disorder.

As used herein, the terms “prevent”, “preventing” and “prevention” refer to the prevention of the development, recurrence or onset of a disease or disorder (e.g., periodontal disease, Gorham-Stout disease, Wilson's disease, chronic otitis media, hypertrophic pulmonary osteoarthropathy or aseptic loosening of joint replacement, or a condition associated therewith) or a symptom thereof in a subject resulting from the administration of a therapy (e.g., prophylactic agent), or the administration of a combination of therapies (e.g., a combination of prophylactic or therapeutic agents).

As used herein, the terms “prophylactic agent” and “prophylactic agents” refer to any agent(s) which can be used in the prevention of a disease or disorder (e.g., periodontal disease, Gorham-Stout disease, Wilson's disease, chronic otitis media, hypertrophic pulmonary osteoarthropathy or aseptic loosening of joint replacement, or a condition associated therewith). In certain embodiments, the term “prophylactic agent” refers to an antibody that immunospecifically binds to integrin αᵥβ₃. In certain other embodiments, the term “prophylactic agent” does not refer an integrin αᵥβ₃ antagonist. In yet other embodiments, the term “prophylactic agents” refers to an integrin αᵥβ₃ antagonist and an agent other than an integrin αᵥβ₃ antagonist. Preferably, a prophylactic agent is an agent which is known to be useful to, or has been or is currently being used to the prevent or impede the onset, development, progression and/or severity of a disease or disorder (e.g., aseptic loosening of joint replacement or conditions associated therewith, or periodontal disease). Prophylactic agents may be characterized as different agents based upon one or more effects that the agents have in vitro and/or in vivo.

As used herein, the term “prophylactically effective amount” refers to that amount of the therapy (e.g., a prophylactic agent) sufficient to result in the prevention of the development, recurrence or onset of a disease or disorder (e.g., periodontal disease, Gorham-Stout disease, Wilson's disease, chronic otitis media, hypertrophic pulmonary osteoarthropathy or aseptic loosening of joint replacement, or a condition associated therewith), or a symptom thereof, or to enhance or improve the prophylactic effects of another therapy (e.g., another prophylactic or therapeutic agent). Examples of suitable dosages of prophylactically or therapeutically effective amounts of agents are given in Section 4.5.2 infra.

As used herein, a “prophylactic protocol” refers to a regimen for dosing and timing the administration of at least one prophylactic agent.

A used herein, a “protocol” includes dosing schedules and dosing regimens. The protocols herein are methods of use and include prophylactic and therapeutic protocols.

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky.

As used herein, the term “small molecules” and analogous terms include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a chimpanzee, a monkey such as a cynomolgous monkey, and a human), and more preferably a human. In one embodiment, the subject is a farm animal (e.g., a horse, pig or bovine), a pet (e.g., a guinea pig, dog or cat), or a laboratory animal (e.g., a mouse or rat). In another embodiment, the subject is a mammal, preferably a human, with aseptic loosening of joint replacement or a condition associated therewith. In another embodiment, the subject is a mammal, preferably a human, with aseptic loosening of hip or knee replacement or a condition associated therewith. In another embodiment, the subject is a mammal, preferably a human, with periodontal disease. In another embodiment, the subject is a mammal, preferably a human, with an increased risk of developing periodontal disease (e.g., a smoker, a subject with poor oral hygiene, a subject with an increased presence of certain gram negative bacteria (e.g., Porphyromonas gingivalis, Prevotella internedia, Bacteroides forsythus, Treponema denticola, and Actinobacillus actinomycetecomitans), a subject taking certain prescription drugs that may lead to gingival overgrowth and inflammation such as antiepileptic drugs (e.g., phenyloin (DILANTIN™)), drugs used in immunosuppressive therapy in transplant patients (e.g., cyclosporin), and various calcium channel blockers used in heart disease). In another embodiment, the subject is a mammal, preferably a human, with Gorham-Stout disease. In another embodiment, the subject is a mammal, preferably a human, with Wilson's disease. In another embodiment, the subject is a mammal, preferably a human, with chronic otitis media. In another embodiment, the subject is a mammal, preferably a human, with hypertrophic pulmonary osteoarthropathy.

As used herein, the term “synergistic” refers to a combination of therapies (e.g., a combination of prophylactic or therapeutic agents) which is more effective than the additive effects of any two or more single agents. A synergistic effect of a combination of therapies (e.g., prophylactic or therapeutic agents) permits the use of lower dosages of at least one of the agents and/or less frequent administration of said therapies (e.g., agents) to a subject with a disease or disorder (e.g., periodontal disease, Gorham-Stout disease, Wilson's disease, chronic otitis media, hypertrophic pulmonary osteoarthropathy or aseptic loosening of joint replacement or a condition associated therewith). The ability to utilize lower dosages of therapies (e.g., prophylactic or therapeutic agents) and/or to administer said therapies (e.g., agents) less frequently reduces the toxicity associated with the administration of said therapies (e.g., agents) to a subject without reducing the efficacy of said agents in the prevention, treatment, management or amelioration of a disease or disorder (e.g., periodontal disease, Gorham-Stout disease, Wilson's disease, chronic otitis media, hypertrophic pulmonary osteoarthropathy or aseptic loosening of joint replacement or a condition associated therewith). In addition, a synergistic effect can result in improved efficacy of therapies (e.g., agents) in the prevention, treatment, management or amelioration of a disease or disorder (e.g., periodontal disease, Gorham-Stout disease, Wilson's disease, chronic otitis media, hypertrophic pulmonary osteoarthropathy or aseptic loosening of joint replacement or a condition associated therewith). Finally, synergistic effect of a combination of therapies (e.g., prophylactic or therapeutic agents) may avoid or reduce adverse or unwanted side effects associated with the use of any single therapy.

As used herein, the terms “therapeutic agent” and “therapeutic agents” refer to any agent(s) which can be used in the treatment, management or amelioration of a disease or disorder (e.g., periodontal disease, Gorham-Stout disease, Wilson's disease, chronic otitis media, hypertrophic pulmonary osteoarthropathy or aseptic loosening of joint replacement or a condition associated therewith) or a symptom thereof. In certain embodiments, the term “therapeutic agent” refers to an integrin αᵥβ₃ antagonist. In certain other embodiments, the term “therapeutic agent” refers does not refer to an integrin αᵥβ₃ antagonist. In yet other embodiments, the term “therapeutic agents” refers to an integrin αᵥβ₃ antagonist and an agent other than an integrin αᵥβ₃ antagonist. Preferably, a therapeutic agent is an agent which is known to be useful for, or has been or is currently being used for the treatment or amelioration of a disease or disorder (e.g., periodontal disease, Gorham-Stout disease, Wilson's disease, chronic otitis media, hypertrophic pulmonary osteoarthropathy or aseptic loosening of joint replacement or a condition associated therewith) or a symptom thereof. Therapeutic agents may be characterized as different agents based upon one or more effects that the agents have in vitro and/or in vivo. For example, a particular anti-inflammatory agent may also be characterized as an anti-arthritic agent.

As used herein, the term “therapeutically effective amount” refers to that amount of a therapy (e.g., therapeutic agent) sufficient to reduce the severity of a disease or disorder (e.g., periodontal disease, Gorham-Stout disease, Wilson's disease, chronic otitis media, hypertrophic pulmonary osteoarthropathy or aseptic loosening of joint replacement or a condition associated therewith) or a symptom thereof, reduce the duration of a disease or disorder (e.g., periodontal disease, Gorham-Stout disease, Wilson's disease, chronic otitis media, hypertrophic pulmonary osteoarthropathy or aseptic loosening of joint replacement or a condition associated therewith) or a symptom thereof, ameliorate of a symptom of a disease or disorder (e.g., periodontal disease, Gorham-Stout disease, Wilson's disease, chronic otitis media, hypertrophic pulmonary osteoarthropathy or aseptic loosening of joint replacement or a condition associated therewith), prevent the advancement of a disease or disorder (e.g., periodontal disease, Gorham-Stout disease, Wilson's disease, chronic otitis media, hypertrophic pulmonary osteoarthropathy or aseptic loosening of joint replacement), cause regression of the disease or disorder (e.g., periodontal disease, Gorham-Stout disease, Wilson's disease, chronic otitis media, hypertrophic pulmonary osteoarthropathy or aseptic loosening of joint replacement), or to enhance or improve the therapeutic effect(s) of another therapy (e.g., another therapeutic agent). In certain embodiments, with respect to the treatment of aseptic loosening of joint replacement, or conditions associated therewith, a therapeutically effective amount refers to the amount of a therapeutic agent that reduces the rate of periprosthetic bone loss in a subject. Preferably, a therapeutically effective amount of a therapeutic agent reduces the e.g., rate of periprosthetic bone loss in a subject by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a control such as PBS, as measured by, for example, the amount of bone-remodeling, osteolysis, linear/volumetric wear, Harris hip score, the Merle d'Aubign and Postel hip score, the McMaster-Toronto Arthritis Patient Preference Disability Questionnaire (MACTAR), the Western Ontario and McMaster University Osteoarthritis Index (WOMAC), measuring bone volume in vivo by microcomputed tomography analysis and bone histomorphometry analysis. In other embodiments, with respect to the treatment of periodontal disease, a therapeutically effective amount refers to the amount of a therapeutic agent that reduces the amount of inflammation of the gums in a subject. Preferably, a therapeutically effective amount of a therapeutic agent reduces the amount of inflammation of the gums in a subject by at least 5%, preferably at least 10%, at least 15%, at least 20%, at least 25%, at least 30%, at least 35%, at least 40%, at least 45%, at least 50%, ate least 55%, at least 60%, at least 65%, at least 70%, at least 75%, at least 80%, at least 85%, at least 90%, at least 95% or at least 99% relative to a control such as PBS, as measured, for example, by the amount of erythematous (redness), edematous (swelling), bleeding, and sensitivity and tenderness of the gingiva. In other embodiments, with respect to the treatment of chronic otitis media, a therapeutically effective amount refers to the amount of a therapeutic agent that reduces the amount of inflammation (in particular, the amount of inflammation in the ear) in a subject. Preferably, a therapeutically effective amount of a therapeutic agent reduces the amount of inflammation in a subject by at least 5%, preferably at least 10%, at least 15%, at least 20%, at least 25%, at least 30%, at least 35%, at least 40%, at least 45%, at least 50%, ate least 55%, at least 60%, at least 65%, at least 70%, at least 75%, at least 80%, at least 85%, at least 90%, at least 95% or at least 99% relative to a control such as PBS, as measured, for example, by the amount of erythematous (redness), edematous (swelling), bleeding, and sensitivity and tenderness of the middle ear. In other embodiments, with respect to the treatment of Gorham-Stout disease and hypertrophic pulmonary osteoarthropathy, a therapeutically effective amount refers to the amount of a therapeutic agent that reduces the amount of bone resportion in a subject. Preferably, a therapeutically effective amount of a therapeutic agent reduces the amount of bone resorption in a subject by at least 5%, preferably at least 10%, at least 15%, at least 20%, at least 25%, at least 30%, at least 35%, at least 40%, at least 45%, at least 50%, ate least 55%, at least 60%, at least 65%, at least 70%, at least 75%, at least 80%, at least 85%, at least 90%, at least 95% or at least 99% relative to a control such as PBS, as measured, for example, by measuring the amount of bone-remodeling or osteolysis by measuring bone volume in vivo by microcomputed tomography analysis or bone histomorphometry analysis.

As used herein, the term “therapeutic protocol” refers to a regimen for dosing and timing the administration of at least one therapeutic agent.

As used herein, the terms “therapies” and “therapy” can refer to any protocol(s), method(s) and/or agent(s) that can be used in the prevention, treatment, management or amelioration of a disease or disorder (e.g., periodontal disease, Gorham-Stout disease, Wilson's disease, chronic otitis media, hypertrophic pulmonary osteoarthropathy or aseptic loosening of joint replacement or a condition associated therewith), or a symptom or condition associated therewith. In certain embodiments, the terms “therapy” and “therapies” refer to analgesics, anti-inflammatory agents, dental preparations, anti-arthritic agents, inhibitors of metalloproteinases, antibiotics (including Periostat™), chelating agents, zinc acetate (Galzin®), bone metabolism regulating agents, hormones, vitamins, immunomodulatory agents, surgery, and/or other therapies useful for the treatment of periodontal disease, Gorham-Stout disease, Wilson's disease, chronic otitis media, hypertrophic pulmonary osteoarthropathy or aseptic loosening of joint replacement or a condition associated therewith known to a physician, nurse or other medical personnel skilled in the art.

As used herein, the terms “treat”, “treatment” and “treating” refer to the reduction or amelioration of the progression, severity and/or duration of a disease or disorder (e.g., periodontal disease, Gorham-Stout disease, Wilson's disease, chronic otitis media, hypertrophic pulmonary osteoarthropathy or aseptic loosening of joint replacement or a condition associated therewith) or the amelioration of a symptom associated said disease or disorder resulting from the administration of at least one therapy (e.g., a prophylactic or therapeutic agent). In specific embodiments, such terms refer to a reduction in the swelling of one or more joints, organs or tissues, or a reduction in the pain associated with inflammation. In other embodiments, such terms refer to a reduction in bone resorption.

### 4. DETAILED DESCRIPTION OF THE INVENTION

The present invention encompasses prophylactic and therapeutic protocols that provide better prophylactic or therapeutic profiles than current single agent therapies or combination therapies for aspetic loosening of joint replacement or conditions associated therewith, or periodontal disease. The invention provides integrin αᵥβ₃ antagonist therapies for the prevention, treatment, management, or amelioration of periodontal disease, Gorham-Stout disease, Wilson's disease, chronic otitis media, hypertrophic pulmonary osteoarthropathy or aseptic loosening of joint replacement or a condition or symptom associated therewith. In particular, the invention provides prophylactic and therapeutic protocols for the prevention, treatment, management or amelioration of periodontal disease, Gorham-Stout disease, Wilson's disease, chronic otitis media, hypertrophic pulmonary osteoarthropathy or aseptic loosening of joint replacement or a symptom or condition associated therewith, comprising administering to a subject in need thereof a prophylactically or therapeutically effective amount of an integrin αᵥβ₃ antagonist (preferably, integrin αᵥβ₃ antibody, more preferably VITAXIN™ or an antigen-binding fragment thereof) alone or in combination with a prophylactically or therapeutically effective amount of at least one other therapy (e.g., at least one other prophylactic or therapeutic agent) other than an integrin αᵥβ₃ antagonist.

The present invention provides pharmaceutical compositions and articles of manufacture comprising an integrin αᵥβ₃ antagonist (preferably, integrin αᵥβ₃ antibody, more preferably VITAXIN™ or an antigen-binding fragment thereof) for use in the prevention, treatment, or management, or amelioration of periodontal disease, Gorham-Stout disease, Wilson's disease, chronic otitis media, hypertrophic pulmonary osteoarthropathy or aseptic loosening of joint replacement or a condition or symptom associated therewith. The present invention also provides pharmaceutical compositions and articles of manufacture comprising an integrin αᵥβ₃ antagonist (preferably integrin αᵥβ₃ antibody, more preferably VITAXIN™ or an antigen binding fragment thereof) and at least one prophylactic or therapeutic agent other than an integrin αᵥβ₃ antagonist for use in the prevention, treatment or amelioration of periodontal disease, Gorham-Stout disease, Wilson's disease, chronic otitis media, hypertrophic pulmonary osteoarthropathy or aseptic loosening of joint replacement or a symptom or condition associated therewith. The present invention also encompasses methods to diagnose, detect and monitor the progression, regression, or stasis of periodontal disease, Gorham-Stout disease, Wilson's disease, chronic otitis media, hypertrophic pulmonary osteoarthropathy or aseptic loosening of joint replacement or a condition associated therewith.

4.1 Integrin αᵥβ₃ Antagonists

Any integrin αᵥβ₃ antagonist well-known to one of skill in the art may be used in the methods and compositions of the invention. The invention encompasses the use of an integrin αᵥβ₃ antagonist in the compositions and methods of the invention. Examples of integrin αᵥβ₃ antagonists include, but are not limited to, proteinaceous agents such as non-catalytic metalloproteinase fragments, RGD peptides, peptide mimetics, fusion proteins, disintegrins or derivatives or analogs thereof, and antibodies that immunospecifically bind to integrin αᵥβ₃, nucleic acid molecules (e.g., nucleic acid molecules encoding antibodies that immunospecifically bind to integrin αᵥβ₃), organic molecules, and inorganic molecules. Non-limiting examples of RGD peptides recognized by integrin αᵥβ₃ include Triflavin. Examples of antibodies that immunospecifically bind to integrin αᵥβ₃ include, but are not limited to, 11D2 (Searle), LM609 (Scripps; International Publication No. WO 89/05155 and U.S. Pat. No. 5,753,230, which is incorporated herein by reference in its entirety), and MEDI-522 (a.k.a. VITAXIN™; MedImmune, Inc., Gaithersburg, Md.; Wu et al., 1998, PNSA USA 95(11):6037-6042; International Publication Nos. WO 98/33919 and WO 00/78815, each of which is incorporated herein by reference in its entirety), 17661-37E and 17661-37E 1-5 (US Biological), MON 2032 (CalTag), ab7166 (BV3) and ab 7167 (BV4) (Abcam), and WOW-I (Kiosses et al., Nature Cell Biology 3:316-320). Non-limiting examples of small molecule peptidometric integrin αᵥβ₃ antagonists include S836 (Searle) and S448 (Searle). Examples of disintegrins include, but are not limited to, Accutin. The invention also encompasses the use of any of the integrin αᵥβ₃ antagonists disclosed in the following U.S. patents and U.S. patent application Publications in the compositions and methods of the invention: U.S. Pat. Nos. 6,344,484; 6,316,412; 6,297,249; 6,294,549; 6,274,620; 6,268,378; 6,232,308; 6,211,184; 6,204,282; 6,193,968; 6,171,588; 6,160,099; 6,153,628; 6,130,231; 6,127,335; 6,100,423; 6,096,707; 6,090,944; 6,066,648; 6,048,861; 6,037,176; 6,017,926; 6,017,925; 5,985,278; 5,981,546; 5,981,478; 5,955,572; 5,952,341; 5,925,655; 5,919,792; 5,877,281; 5,852,210; 5,849,865; 5,849,692; 5,830,678; 5,843,906; 5,843,774; 5,817,457; 5,807,819; 5,792,745; 5,780,426; 5,773,646; 5,773,644; 5,773,412; 5,770,565; 5,767,071; 5,766,591; 5,760,029; 5,760,028; 5,759,996; 5,753,230; 5,710,159; 5,705,481; 5,693,612; 5,681,820; 5,652,110; 5,652,109; 5,578,704; 5,589,570; 5,523,209; 5,498,694; 5,478,725; 5,306,620; 5,262,520; 5,204,445; 5,196,511; 5,190,873; 5,149,780; and U.S. Patent Application Publication Nos. 20020019402; 20020019387; 20020010176; 20020001840; 20010053853; 20010044535; 20010023242; 20010016645; 20010011125; and 20010001309, which are all herein incorporated by reference in their entireties.

In certain embodiments, an integrin αᵥβ₃ antagonist is a small organic molecule. In other embodiments, an integrin αᵥβ₃ antagonist is not a small organic molecule. In certain other embodiments, an integrin αᵥβ₃ antagonist is an antibody that immunospecifically binds to integrin αᵥβ₃. In other embodiments, an integrin αᵥβ₃ antagonist is not an antibody that immunospecifically binds to integrin αᵥβ₃. In a specific embodiment, an integrin αᵥβ₃ antagonist is VITAXIN™, a derivative, analog, or antigen-binding fragment thereof. In other embodiments, an integrin αᵥβ₃ antagonist is an antibody other than VITAXIN™ or an antigen-binding fragment thereof, that imunospecifically binds to integrin αᵥβ₃.

In a preferred embodiment, integrin αᵥβ₃ antagonists inhibit or reduce angiogenesis. In particular embodiments, integrin αᵥβ₃ antagonists inhibit or reduce angiogenesis in a subject by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% relative to a control such as PBS, as measured by, for example, changes in regional blood volume using dynamic susceptibility contrast-enhanced MRI.

In a preferred embodiment, proteins, polypeptides or peptides (including antibodies and fusion proteins) that are utilized as integrin αᵥβ₃ antagonists are derived from the same species as the recipient of the proteins, polypeptides or peptides so as to reduce the likelihood of an immune response to those proteins, polypeptides or peptides. In another preferred embodiment, when the subject is a human, the antibodies that are utilized as integrin αᵥβ₃ antagonists are human or humanized.

In accordance with the invention, an integrin αᵥβ₃ antagonist are administered to a subject with periodontal disease, Gorham-Stout disease, Wilson's disease, chronic otitis media, hypertrophic pulmonary osteoarthropathy or aseptic loosening of joint replacement or a symptom or condition associated therewith prior to, subsequent to, or concurrently with at least one other therapy (e.g., a prophylactic or therapeutic agent), preferably other therapies that have been used, are currently being used or are known to be useful in the prevention or treatment of said periodontal disease, Gorham-Stout disease, Wilson's disease, chronic otitis media, hypertrophic pulmonary osteoarthropathy or aseptic loosening of joint replacement or a symptom or condition associated therewith.

Nucleic acid molecules encoding proteins, polypeptides, or peptides that function as integrin αᵥβ₃ antagonists, or proteins, polypeptides, or peptides that function as integrin αᵥβ₃ antagonists can be administered to a subject with periodontal disease, Gorham-Stout disease, Wilson's disease, chronic otitis media, hypertrophic pulmonary osteoarthropathy or aseptic loosening of joint replacement or a symptom or condition associated therewith, in accordance with the methods of the invention. Further, nucleic acid molecules encoding derivatives, analogs, fragments or variants of proteins, polypeptides, or peptides that function as integrin αᵥβ₃ antagonists, or derivatives, analogs, fragments or variants of proteins, polypeptides, or peptides that function as integrin αᵥβ₃ antagonists can be administered to a subject with periodontal disease, Gorham-Stout disease, Wilson's disease, chronic otitis media, hypertrophic pulmonary osteoarthropathy or aseptic loosening of joint replacement or a condition associated therewith in accordance with the methods of the invention. Preferably, such derivatives, analogs, variants and fragments retain the integrin αᵥβ₃ antagonist activity of the full-length wild-type protein, polypeptide, or peptide.

4.1.1 Antibodies that Immunospecifically Bind to Intefrin αᵥβ₃

It should be recognized that antibodies that immunospecifically bind to integrin αᵥβ₃ and function as antagonists are known in the art. Examples of known antibodies that immunospecifically bind to integrin αᵥβ₃ include, but are not limited to, 11D2 (Searle), the murine monoclonal LM609 (Scripps; International Publication No. WO 89/05155, and U.S. Pat. No. 5,753,230, which are incorporated herein by reference in their entireties), the humanized monoclonal antibody MEDI-522 (a.k.a. VITAXIN™, Medlmmune, Inc., Gaithersburg, Md.; Wu et al., 1998, PNAAS USA 95(11):6037-6042; International Publication No. WO 98/33919 and WO 00/78815; each of which is incorporated herein by reference in its entirety), and 17661-37E and 17661-37E 1-5 (US Biological), MON 2032 (CalTag), ab7166 (BV3) and ab 7167 (BV4) (Abcam), and WOW-1 (Kiosses et al., Nature Cell Biology 3:316-320).

The antibodies that immunospecifically bind to integrin αᵥβ₃ may be from any animal origin including birds and mammals (e.g., human, murine, donkey, sheep, rabbit, goat, guinea pig, camel, horse, or chicken). Preferably, the antibodies that immunospecifically bind to integrin αᵥβ₃ are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies that immunospecifically bind to integrin αᵥβ₃ may be monospecific, bispecific, trispecific or of greater multispecificity. Multispecific antibodies may be specific for different epitopes of integrin αᵥβ₃ or may be specific for both an integrin αᵥβ₃ epitope as well as for a heterologous epitope, such as a heterologous polypeptide or solid support material. See, e.g., International Publication Nos. WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

The present invention encompasses the use of antibodies that have a high binding affinity for integrin αᵥβ₃. In a specific embodiment, an antibody that immunospecifically binds to integrin αᵥβ₃ has an association rate constant or kₒₙ rate (antibody (Ab)+antigen (Ag)^{k}^{ₒₙ}←Ab−Ag) of at least 10⁵M⁻¹s⁻¹, at least 5×10⁵M⁻s⁻¹, at least 10⁶M⁻¹s⁻¹, at least 5×10⁶M⁻¹s⁻¹, at least 10⁷M⁻¹s⁻¹, at least 5×10⁷M⁻¹s⁻¹, or at least 10⁸M⁻¹s⁻¹. In a preferred embodiment, an antibody that immunospecifically binds to integrin αᵥβ₃ has a kₒₙ of at least 2×10⁵M⁻¹s⁻¹, at least 5×10⁵M⁻¹s⁻¹, at least 10⁶M⁻¹s⁻¹, at least 5×10⁶M⁻¹s⁻¹, at least 10⁷M⁻¹s⁻¹, at least 5×10⁷M⁻¹s⁻¹, or at least 10⁸M⁻¹s⁻¹.

In another embodiment, an antibody that immunospecifically binds to integrin α_{vβ}₃ has a k_{off} rate (antibody (Ab)+antigen (Ag)^{k}^{_{off}}←Ab−Ag) of less than 10⁻¹s⁻¹, less than 5×10⁻¹s⁻¹, less than 10⁻²s⁻¹, less than 5×10⁻²s⁻¹, less than 10⁻³s⁻¹, less than 5×10⁻³s⁻¹, less than 10⁻⁴s⁻¹, less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰⁻¹s⁻¹. In a preferred embodiment, an antibody that immunospecifically binds to integrin αᵥβ₃ has a kₒₙ, of less than 5×10⁻⁴s⁻¹, less than 10⁻⁵s⁻¹, less than 5×10⁻⁵s⁻¹, less than 10⁻⁶s⁻¹, less than 5×10⁻⁶s⁻¹, less than 10⁻⁷s⁻¹, less than 5×10⁻⁷s⁻¹, less than 10⁻⁸s⁻¹, less than 5×10⁻⁸s⁻¹, less than 10⁻⁹s⁻¹, less than 5×10⁻⁹s⁻¹, or less than 10⁻¹⁰s⁻¹.

In another embodiment, an antibody that immunospecifically binds to integrin αᵥβ₃ has an affinity constant or Kₐ (kₒₙ/k_{off}) of at least 10²M⁻¹, at least 5×10²M⁻¹, at least 10³M⁻¹, at least 5×10³M⁻¹, at least 10⁴M⁻¹, at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹M⁻¹, at least 5×10⁹M⁻¹, at least 10¹⁰M⁻¹, at least 5×10¹M⁻¹, at least 10¹¹M⁻¹, at least 5×10¹¹M⁻¹, at least 10¹²M⁻¹, at least 5×10¹²M, at least 10¹³M⁻¹, at least 5×10¹³M⁻¹, at least 10¹⁴M⁻¹, at least 5×10¹⁴M⁻¹, at least 10¹⁵M⁻¹, or at least 5×10¹⁵M⁻¹. In yet another embodiment, an antibody that immunospecifically binds to integrin αᵥβ₃ has a dissociation constant or K_{d} (k_{off}/kₒₙ) of less than 10⁻²M, less than 5×10⁻²M, less than 10⁻³M, less than 5×10⁻³M, less than 10⁻⁴M, less than 5×10⁻⁴M, less than 10⁻⁵M, less than 5×10⁻⁵M, less than 10⁻⁶M, less than 5×10⁻⁶M, less than 10⁻⁷M, less than 5×10⁻⁷M, less than 10⁻⁸M, less than 5×10⁻⁸M, less than 10⁻⁹M, less than 5×10⁻⁹M, less than 10⁻¹⁰M, less than 5×10⁻¹⁰M, less than 10⁻¹¹M, less than 5×10⁻¹¹M, less than 10⁻¹²M, less than 5×10⁻¹²M, less than 10⁻¹³M, less than 5×10⁻¹³M, less than 10⁻¹⁴M, less than 5×10⁻¹⁴M, less than 10⁻¹⁵M, or less than 5×10⁻¹⁵M.

In a specific embodiment, an antibody that immunospecifically binds to integrin αᵥβ₃ is LM609 or an antigen-binding fragment thereof (e.g., at least one complementarity determining region (CDRs) of LM609). LM609 has the amino acid sequence disclosed, e.g., in International Publication No. WO 89/05155 (which is incorporated herein by reference in its entirety), or the amino acid sequence of the monoclonal antibody produced by the cell line deposited with the American Type Culture Collection (ATCC™), 10801 University Boulevard, Manassas, Va. 20110-2209 as Accession Number HB 9537. In an alternative embodiment, an antibody that immunospecifically binds to integrin αᵥβ₃ is not LM609 or an antigen-binding fragment of LM609.

In a preferred embodiment, an antibody that immunospecifically binds to integrin αᵥβ₃ is VITAXIN™ or an antibody-binding fragment thereof (e.g., at least one CDR of VITAXIN™). VITAXIN™ is disclosed, e.g., in International Publication No. WO 98/33919 and WO 00/78815, U.S. application Ser. No. 09/339,222, and U.S. Pat. No. 5,753,230, each of which is incorporated herein by reference in its entirety. In an alternative embodiment, an antibody that immunospecifically binds to integrin αᵥβ₃ is an antibody other than VITAXIN™ or an antigen-binding fragment of VITAXIN™.

The present invention also encompasses the use of antibodies that immunospecifically bind integrin αᵥβ₃, said antibodies comprising a variable heavy (“VH”) domain having an amino acid sequence of the VH domain for LM609 or VITAXIN™. The present invention also encompasses the use of antibodies that immunospecifically bind to integrin αᵥβ₃, said antibodies comprising a VH CDR having an amino acid sequence of any one of the VH CDRs listed in Table 1 infra.

TABLE 1
CDR Sequences Of LM609

| CDR | Sequence | SEQ ID NO: |
|---|---|---|
| VH1 | SYDMS | 1 |
| VH2 | KVSSGGG | 2 |
| VH3 | HNYGSFAY | 3 |
| VL1 | QASQSISNHLH | 4 |
| VL2 | YRSQSIS | 5 |
| VL3 | QQSGSWPHT | 6 |

In one embodiment, antibodies that immunospecifically bind to integrin αᵥβ₃ comprise a VH CDR1 having the amino acid sequence of SEQ ID NO:1. In another embodiment, antibodies that immunospecifically bind to integrin αᵥβ₃ comprise a VH CDR2 having the amino acid sequence of SEQ ID NO:2. In another embodiment, antibodies that immunospecifically bind to integrin αᵥβ₃ comprise a VH CDR3 having the amino acid sequence of SEQ ID NO:3. In another embodiment, antibodies that immunospecifically bind to integrin αᵥβ₃ comprise a combination of VH CDR1 having an amino acid sequence of SEQ ID NO:1 and VH CDR2 having an amino acid sequence of SEQ ID NO:2. In another embodiment, antibodies that immunospecifically bind to integrin αᵥβ₃ comprise a combination of VH CDR1 having an amino acid sequence of SEQ ID NO:1 and VH CDR3 having an amino acid sequence of SEQ ID NO:3. In another embodiment, antibodies that immunospecifically bind to integrin αᵥβ₃ comprise a combination of VH CDR2 having an amino acid sequence of SEQ ID NO:2 and VH CDR3 having an amino acid sequence of SEQ ID NO:3. In a preferred embodiment, antibodies that immunospecifically bind to integrin αᵥβ₃ comprise a VH CDR1 having the amino acid sequence of SEQ ID NO:1, a VH CDR2 having the amino acid sequence of SEQ ID NO:2, and a VH CDR3 having the amino acid sequence of SEQ ID NO:3.

The present invention encompasses the use of antibodies that immunospecifically bind to integrin αᵥβ₃, said antibodies comprising a variable light (“VL”) domain having an amino acid sequence of the VL domain for LM609 or VITAXIN™. In another embodiment, antibodies that immunospecifically bind to integrin αᵥβ₃ comprise a combination of VL CDR1 having an amino acid sequence of SEQ ID NO:4 and VL CDR2 having an amino acid sequence of SEQ ID NO:5. In another embodiment, antibodies that immunospecifically bind to integrin αᵥβ₃ comprise a combination of VL CDR1 having an amino acid sequence of SEQ ID NO:4 and VL CDR3 having an amino acid sequence of SEQ ID NO:6. In another embodiment, antibodies that immunospecifically bind to integrin αᵥβ₃ comprise a combination of VL CDR2 having an amino acid sequence of SEQ ID NO:5 and VL CDR3 having an amino acid sequence of SEQ ID NO:6. The present invention encompasses the use of antibodies that immunospecifically bind to integrin αᵥβ₃ said antibodies comprising a VL CDR having an amino acid sequence of any one of the VL CDRs listed in Table 1.

In one embodiment, antibodies that immunospecifically bind to integrin αᵥβ₃ comprise a VL CDR1 having the amino acid sequence of SEQ ID NO:4. In another embodiment, antibodies that immunospecifically bind to integrin αᵥβ₃ comprise a VL CDR2 having the amino acid sequence of SEQ ID NO:5. In another embodiment, antibodies that immunospecifically bind to integrin αᵥβ₃ comprise a VL CDR3 having the amino acid sequence of SEQ ID NO:6. In a preferred embodiment, antibodies that immunospecifically bind to integrin αᵥβ₃ comprise a VL CDR1 having the amino acid sequence of SEQ ID NO:4, a VL CDR2 having the amino acid sequence of SEQ ID NO:5, and a VL CDR3 having the amino acid sequence of SEQ ID NO:6.

The present invention encompasses the use of antibodies that immunospecifically bind to integrin αᵥβ₃, said antibodies comprising a VH domain disclosed herein combined with a VL domain disclosed herein, or other VL domain. The present invention encompasses the use of antibodies that immunospecifically bind to integrin αᵥβ₃, said antibodies comprising a VL domain disclosed herein combined with a VH domain disclosed herein, or other VH domain.

The present invention encompasses the use of antibodies that immunospecifically bind to integrin αᵥβ₃, said antibodies comprising at least one VH CDR and at least one VL CDR listed in Table 1. In particular, the invention encompasses the use of an antibody that immunospecifically binds to integrin αᵥβ₃, said antibody comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3, or any combination thereof of the VH CDRs and VL CDRs listed in Table 1 supra.

In one embodiment, an antibody that immunospecifically binds to integrin αᵥβ₃ comprises a VH CDR1 having the amino acid sequence of SEQ ID NO:1 and a VL CDR1 having the amino acid sequence of SEQ ID NO:4. In another embodiment, an antibody that immunospecifically binds to integrin αᵥβ₃ comprises a VH CDR1 having the amino acid sequence of SEQ ID NO:1 and a VL CDR2 having the amino acid sequence of SEQ ID NO:5. In another embodiment, an antibody that immunospecifically binds to integrin αᵥβ₃ comprises a VH CDR1 having the amino acid sequence of SEQ ID NO:1 and a VL CDR3 having the amino acid sequence of SEQ ID NO:6.

In another embodiment, an antibody that immunospecifically binds to integrin αᵥβ₃ comprises a VH CDR2 having the amino acid sequence of SEQ ID NO:2 and a VL CDR1 having the amino acid sequence of SEQ ID NO:4. In another embodiment, an antibody that immunospecifically binds to integrin αᵥβ₃ comprises a VH CDR2 having the amino acid sequence of SEQ ID NO:2 and a VL CDR2 having the amino acid sequence of SEQ ID NO:5. In another embodiment, an antibody that immunospecifically binds to integrin αᵥβ₃ comprises a VH CDR2 having the amino acid sequence of SEQ ID NO:2 and a VL CDR3 having the amino acid sequence of SEQ ID NO:6.

In another embodiment, an antibody that immunospecifically binds to integrin αᵥβ₃ comprises a VH CDR3 having the amino acid sequence of SEQ ID NO:3 and a VL CDR1 having the amino acid sequence of SEQ ID NO:4. In another embodiment, an antibody that immunospecifically binds to integrin αᵥβ₃ comprises a VH CDR3 having the amino acid sequence of SEQ ID NO:3 and a VL CDR2 having the amino acid sequence of SEQ ID NO:5. In a preferred embodiment, an antibody that immunospecifically binds to integrin αᵥβ₃ comprises a VH CDR3 having the amino acid sequence of SEQ ID NO:3 and a VL CDR3 having the amino acid sequence of SEQ ID NO:6.

The present invention encompasses the use of a nucleic acid molecule(s), generally isolated, encoding an antibody that immunospecifically binds to integrin αᵥβ₃. In a specific embodiment, an isolated nucleic acid molecule(s) encodes an antibody that immunospecifically binds to integrin αᵥβ₃, said antibody having the amino acid sequence of LM609 or VITAXIN™.

In one embodiment, an isolated nucleic acid molecule encodes an antibody that immunospecifically binds to integrin αᵥβ₃, said antibody comprising a VH domain having the amino acid sequence of the VH domain of LM609 or VITAXIN™. In another embodiment, an isolated nucleic acid molecule encodes an antibody that immunospecifically binds to integrin αᵥβ₃, said antibody comprising a VH domain having the amino acid sequence of the VH domain of the monoclonal antibody produced by the cell line deposited with the ATCC™ as Accession Number HB 9537. In another embodiment, an isolated nucleic acid molecule encodes an antibody that immunospecifically binds to integrin αᵥβ₃, said antibody comprising a VH CDR1 having the amino acid sequence of the VH CDR1 listed in Table 1. In another embodiment, an isolated nucleic acid molecule encodes an antibody that immunospecifically binds to integrin αᵥβ₃, said antibody comprising a VH CDR2 having the amino acid sequence of the VH CDR2 listed in Table 1. In yet another embodiment, an isolated nucleic acid molecule encodes an antibody that immunospecifically binds to integrin αᵥβ₃, said antibody comprising a VH CDR3 having the amino acid sequence of the VH CDR3 listed in Table 1.

In one embodiment, an isolated nucleic acid molecule encodes an antibody that immunospecifically binds to integrin αᵥβ₃, said antibody comprising a VL domain having the amino acid sequence of the VL domain of LM609 or VITAXIN™. In another embodiment, an isolated nucleic acid molecule encodes an antibody that immunospecifically binds to integrin αᵥβ₃, said antibody comprising a VL domain having the amino acid sequence of the VL domain of the monoclonal antibody produced by the cell line deposited with the ATCC™ as Accession Number HB 9537. In another embodiment, an isolated nucleic acid molecule encodes an antibody that immunospecifically binds to integrin αᵥβ₃, said antibody comprising a VL CDR1 having the amino acid sequence of the VL CDR1 listed in Table 1. In another embodiment, an isolated nucleic acid molecule encodes an antibody that immunospecifically bind to integrin αᵥβ₃, said antibody comprising a VL CDR2 having the amino acid sequence of the VL CDR2 listed in Table 1. In yet another embodiment, an isolated nucleic acid molecule encodes an antibody that immunospecifically binds to integrin αᵥβ₃, said antibody comprising a VL CDR3 having the amino acid sequence of the VL CDR3 listed in Table 1.

In another embodiment, an isolated nucleic acid molecule encodes an antibody that immunospecifically binds to integrin αᵥβ₃, said antibody comprising a VH domain having the amino acid sequence of the VH domain of LM609 or VITAXIN™ and a VL domain having the amino acid sequence of the VL domain of LM609 or VITAXIN™. In another embodiment, an isolated nucleic acid molecule encodes an antibody that immunospecifically binds to integrin αᵥβ₃, said antibody comprising a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3, or any combination thereof having an amino acid sequence listed in Table 1.

The present invention encompasses the use of antibodies that immunospecifically bind to integrin αᵥβ₃, said antibodies comprising derivatives of the VH domains, VH CDRs, VL domains, or VL CDRs described herein that immunospecifically bind to integrin αᵥβ₃ Standard techniques known to those of skill in the art can be used to introduce mutations (e.g., substitutions, deletions and/or additions) in the nucleotide sequence encoding an antibody of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at at least one predicted non-essential amino acid residue (e.g., an amino acid residue which is not critical for the antibody to immunospecifically bind to integrin αᵥβ₃). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

The present invention encompasses the use of antibodies that immunospecifically bind to integrin αᵥβ₃, said antibodies comprising the amino acid sequence of LM609 or VITAXIN™ with at least one amino acid residue substitution in the variable light (VL) domain and/or variable heavy (VH) domain. The present invention encompasses the use of antibodies that immunospecifically bind to integrin αᵥβ₃, said antibodies comprising the amino acid sequence of LM609 or VITAXIN™ with at least one amino acid residue substitution in at least one VL CDR and/or at least one VH CDR. The antibody generated by introducing substitutions in the VH domain, VH CDRs, VL domain and/or VL CDRs of LM609 or VITAXIN™ can be tested in vitro and in vivo, for example, for its ability to bind to integrin αᵥβ₃ (by, e.g., immunoassays including, but not limited to ELISAs and BIAcore), or for its ability to prevent, treat or ameliorate a symptom associated with periodontal disease, Gorham-Stout disease, Wilson's disease, chronic otitis media, hypertrophic pulmonary osteoarthropathy or aseptic loosening of joint replacement.

In a specific embodiment, an antibody that immunospecifically binds to integrin αᵥβ₃ is encoded by a nucleic acid sequence comprising a nucleotide sequence that hybridizes to the nucleotide sequence encoding the monoclonal antibody produced by the cell line deposited with the ATCC™ as Accession Number HB 9537 under stringent conditions, e.g., hybridization to filter-bound DNA in 6× sodium chloride/sodium citrate (SSC) at about 45 C followed by one or more washes in 0.2×SSC/0.1% SDS at about 50-65 C, under highly stringent conditions, e.g. hybridization to filter-bound nucleic acid in 6×SSC at about 45 C followed by one or more washes in 0.1×SSC/0.2% SDS at about 68 C, or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In a specific embodiment, an antibody that immunospecifically binds to integrin αᵥβ₃ is encoded by a nucleic acid sequence comprising a nucleotide sequence that hybridizes to the nucleotide sequence encoding the LM609 or VITAXIN™ under stringent conditions. In another embodiment, an antibody that immunospecifically binds to integrin CO₃ comprises an amino acid sequence of a VH domain or an amino acid sequence of a VL domain encoded by a nucleic acid sequence comprising a nucleotide sequence that hybridizes to the nucleotide sequence encoding the VH or VL domains of LM609 or VITAXIN™ under stringent conditions. In another embodiment, an antibody that immunospecifically binds to integrin αᵥβ₃ comprises an amino acid sequence of a VH domain and an amino acid sequence of a VL domain encoded by a nucleic acid sequence comprising a nucleotide sequence that hybridizes to the nucleotide sequence encoding the VH and VL domains of LM609 or VITAXIN™ under stringent conditions. In another embodiment, an antibody that immunospecifically binds to integrin αᵥβ₃ comprises an amino acid sequence of a VH CDR or an amino acid sequence of a VL CDR encoded by a nucleic acid sequence comprising a nucleotide sequence that hybridizes to the nucleotide sequence encoding any one of the VH CDRs or VL CDRs listed in Table 1 under stringent conditions.

In another embodiment, an antibody that immunospecifically binds to integrin αᵥβ₃ comprises an amino acid sequence of a VH CDR or an amino acid sequence of a VL CDR encoded by a nucleic acid sequence comprising a nucleotide sequence that hybridizes to the nucleotide sequence encoding any one of VH CDRs or VL CDRs of the monoclonal antibody produced by the cell line deposited with the ATCC™ as Accession Number HB 9537 under stringent conditions. In another embodiment, an antibody that immunospecifically binds to integrin αᵥβ₃ comprises an amino acid sequence of a VH CDR and an amino acid sequence of a VL CDR encoded by a nucleic acid sequence comprising a nucleotide sequence that hybridizes to the nucleotide sequence encoding any one of the VH CDRs and VL CDRs listed in Table 1 under stringent conditions. In another embodiment, an antibody that immunospecifically binds to integrin αᵥβ₃ comprises an amino acid sequence of a VH CDR and an amino acid sequence of a VL CDR encoded by a nucleic acid sequence comprising a nucleotide sequence that hybridizes to the nucleotide sequence encoding the monoclonal antibody produced by the cell line deposited with the ATCC™ as Accession Number HB 9537 under stringent conditions.

In a specific embodiment, an antibody that immunospecifically binds to integrin αᵥβ₃ comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% of identical to the amino acid sequence of the monoclonal antibody produced by the cell line deposited with the ATCC™ as Accession Number HB 9537. In another embodiment, an antibody that immunospecifically binds to integrin αᵥβ₃ comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of VITAXIN™. The determination of percent identity of two amino acid sequences can be determined by any method known to one skilled in the art, including BLAST protein searches.

In another embodiment, an antibody that immunospecifically binds to integrin αᵥβ₃ comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of VITAXIN™. In another embodiment, an antibody that immunospecifically binds to integrin αᵥβ₃ comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of the monoclonal antibody produced by the cell line deposited with the ATCC™ as Accession Number HB 9537.

In another embodiment, an antibody that immunospecifically binds to integrin αᵥβ₃ comprises an amino acid sequence of at least one VH CDR that is at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VH CDRs listed in Table 1. In another embodiment, an antibody that immunospecifically binds to integrin αᵥβ₃ comprises an amino acid sequence of at least one VH CDR that is at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VH CDRs of the monoclonal antibody produced by the cell line deposited with the ATCC™ as Accession Number HB 9537.

In another embodiment, an antibody that immunospecifically binds to integrin αᵥβ₃ comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of VITAXIN™. In another embodiment, an antibody that immunospecifically binds to integrin αᵥβ₃ comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of the monoclonal antibody produced by the cell line deposited with the ATCC™ as Accession Number HB 9537.

In another embodiment, an antibody that immunospecifically binds to integrin αᵥβ₃ comprises an amino acid sequence of at least one VL CDR that is at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 1. In another embodiment, an antibody that immunospecifically binds to integrin αᵥβ₃ comprises an amino acid sequence of at least one VL CDR that is at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs of the monoclonal antibody produced by the cell line deposited with the ATCC™ as Accession Number HB 9537.

The present invention encompasses antibodies that compete with an antibody described herein for binding to integrin αᵥβ₃. In a specific embodiment, the present invention encompasses antibodies that compete with LM609 or an antigen-binding fragment thereof for binding to integrin αᵥβ₃. In a preferred embodiment, the present invention encompasses antibodies that compete with VITAXIN™ or an antigen-binding fragment thereof for binding to integrin αᵥβ₃. In specific embodiments, competitive assay systems are used to determine competition. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety). In one embodiment, the competitive binding assay is an enzyme-linked immunosorbent assay (ELISA).

The present invention also encompasses proteins, polypeptides, or peptides comprising a VH domain that competes with the VH domain of LM609 or VITAXIN™ or a protein, polypeptide, or peptide comprising a VH domain of LM609 or VITAXIN™ for binding to integrin αᵥβ₃. The present invention also encompasses proteins, polypeptides, or peptides comprising a VL domain that competes with a VL domain of LM609 or VITAXIN™ or a protein, polypeptide, or peptide comprising a VL domain of LM609 or VITAXIN™ for binding to integrin αᵥβ₃.

The present invention also encompasses proteins, polypeptides, or peptides comprising at least one VH CDR that competes with a VH CDR listed in Table 1 or a protein, polypeptide, or peptide comprising a VH CDR listed in Table 1 for binding to integrin αᵥβ₃, or a VH CDR of the monoclonal antibody produced by the cell line deposited with the ATCC™ as Accession Number HB 9537 for binding to integrin αᵥβ₃. The present invention also encompasses proteins, polypeptides, or peptides comprising at least one VL CDR that competes with a VL CDR listed in Table 1 or a protein, polypeptide or peptide comprising a VL CDR listed in Table 1 for binding to integrin αᵥβ₃, or a VL CDR of the monoclonal antibody produced by the cell line deposited with the ATCC™ as Accession Number HB 9537 for binding to integrin αᵥβ₃ Antibodies that immunospecifically bind to integrin αᵥβ₃ include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain at least one non-classical amino acids.

The present invention also provides antibodies that immunospecifically bind to integrin αᵥβ₃, said antibodies comprising a framework region known to those of skill in the art. Preferably, the fragment region of an antibody of the invention is human. In a specific embodiment, an antibody that immunospecifically binds to integrin αᵥβ₃ comprises the framework region of VITAXIN™.

The present invention also encompasses antibodies that immunospecifically bind to integrin αᵥβ₃, said antibodies comprising the amino acid sequence of VITAXIN™ with at least one mutation (e.g., at least one amino acid substitution) in the framework regions. In certain embodiments, antibodies that immunospecifically bind to integrin αᵥβ₃ comprise the amino acid sequence of VITAXIN™ with at least one amino acid residue substitution in the framework region of the VH and/or VL domains.

The present invention also encompasses antibodies that immunospecifically bind to integrin αᵥβ₃, said antibodies comprising the amino acid sequence of VITAXIN™ with at least one mutation (e.g., at least one amino acid residue substitution) in the variable and framework regions.

The present invention also provides antibodies of the invention that comprise constant regions known to those of skill in the art. Preferably, the constant regions of an antibody of the invention are human.

The present invention encompasses the use of fusion proteins comprising an antibody that immunospecifically binds to integrin αᵥβ₃ and a heterologous polypeptide. Preferably, the heterologous polypeptide that the antibody is fused to is useful for targeting the antibody to platelets, monocytes, macrophages, endothelial cells, osteoclasts, activated T cells, and/or B cells.

4.1.1.1 Antibodies Having Increased Half-Lives That Immunospecifically Bind to Integrin αᵥβ₃

The present invention provides for antibodies that immunospecifically bind to integrin αᵥβ₃ which have a extended half-life in vivo. In particular, the present invention provides antibodies that immunospecifically bind to integrin αᵥβ₃ which have a half-life in an animal, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies, single chain antibodies and Fab fragments) in vivo, for example, inert polymer molecules such as high molecular weight polyethyleneglycol (PEG) can be attached to the antibodies with or without a multifunctional linker either through site-specific conjugation of the PEG to the - or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods known to those of skill in the art, for example, by immunoassays described herein.

Antibodies having an increased half-life in vivo can also be generated introducing at least one amino acid modification (i.e., a substitution, insertion or deletion) into an IgG constant domain, or FcRn binding fragment thereof (preferably a Fc or hinge-Fc domain fragment). See, e.g., International Publication Nos. WO 98/23289; and WO 97/34631; and U.S. Pat. No. 6,277,375, each of which is incorporated herein by reference in its entirety.

Further, antibodies can be conjugated to albumin in order to make the antibody or antibody fragment more stable in vivo or have a longer half-life in vivo. The techniques well-known in the art, see, e.g., International Publication Nos. WO 93/15199, WO 93/15200, and WO 01/77137, and European Patent No. EP 413,622, all of which are incorporated herein by reference.

4.1.1.2 Antibody Conjugates

The present invention provides of antibodies or antibody fragments that immunospecifically binds to integrin αᵥβ₃ recombinantly fused or chemically conjugated (including both covalent and non-covalent conjugations) to another a heterologous polypeptide, an antibody or an antibody fragment, a marker sequence, a diagnostic agent, a therapeutic moiety, a radioactive metal ion, a polymer, albumin, and a solid support. In a particular embodiment, the present invention provides of antibodies or fragments thereof that immunospecifically binds to integrin αᵥβ₃ recombinantly fused or chemically conjugated (including both covalent and non-covalent conjugations) to a heterologous polypeptide or protein (or fragment thereof, preferably to a polypepetide of at least 10, at least 20, at least 30, at least 40, at least 50, at least 60, at least 70, at least 80, at least 90 or at least 100 amino acids), to generate fusion proteins. In particular, the invention provides fusion proteins comprising an antigen-binding fragment of an antibody described herein (e.g., a Fab fragment, Fd fragment, Fv fragment, F(ab)₂ fragment, a VH domain, a VH CDR, a VL domain or a VL CDR) and a heterologous polypeptide or protein. Preferably, the heterologous polypeptide that the antibody or antibody fragment is fused to is useful for targeting the antibody to endothelial cells, B cells, osteoclasts or activated T cells. For example, an antibody that immunospecifically binds to a cell surface receptor expressed by a particular cell type (e.g., an endothelial cell, a B cell, an osteoclast, or an activated T cell) may be fused or conjugated to an antibody or antibody fragment of the invention. Methods for fusing or conjugating polypeptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references incorporated by reference in their entireties).

Additional fusion proteins of anti-integrin αᵥβ₃ antibodies, may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or antibody fragments, or the encoded antibodies or antibody fragments, may be altered by being subjected to random mutagenesis by error-prone PCR, random nucleotide insertion or other methods prior to recombination. A polynucleotide encoding an antibody or antibody fragment that immunospecifically binds to integrin αᵥβ₃ may be recombined with at least one component, motif, section, part, domain, fragment, etc. of at least one heterologous molecule.

Moreover, the antibodies or antibody fragments can be fused to marker sequences, such as a peptide to facilitate purification. In preferred embodiments, the marker amino acid sequence is a hexa-histidine peptide, such as the tag provided in a pQE vector (QIAGEN™, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, antibodies of the present invention or fragments or variants thereof conjugated to a diagnostic or detectable agent. Such antibodies can be useful for monitoring or prognosing the development or progression of a disease or disorder as part of a clinical testing procedure, such as determining the efficacy of a particular therapy. Such diagnosis and detection can be accomplished by coupling the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ²³¹I, ¹²¹I,), carbon (¹⁴C), sulfur (³⁵S), tritium (3H), indium (¹¹⁵In, ¹¹³In, ¹¹²In, ¹¹¹In,), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴² Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹ Cr, ⁵⁴Mn, ⁷⁵Se, ¹¹³Sn, and ¹¹⁷Tin; positron emitting metals using various positron emission tomographies, non-radioactive paramagnetic metal ions, and molecules that are radiolabelled or conjugated to specific radioisotopes.

The present invention further encompasses uses of antibodies or antibody fragments conjugated to a therapeutic moiety. An antibody or antibody fragment may be conjugated to a therapeutic moiety such as a cytotoxin, e.g., a cytostatic or cytocidal agent, a therapeutic moiety or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic moieties include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), and Mohammad et al., Int. J. Oncol. 15:367-72 (1999), all of which are incorporated herein by reference), anti-mitotic agents (e.g., vincristine and vinblastine), hormones (e.g., glucocorticoids, progestatins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)), and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459), farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305), topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin; bulgarein; DNA minor groove binders such as Hoescht dye 33342 and Hoechst dye 33258; nitidine; fagaronine; epiberberine; coralyne; beta-lapachone; BC-4-1;

and pharmaceutically acceptable salts, solvates, clathrates, and prodrugs thereof. See, e.g., Rothenberg, M. L., Annals of Oncology 8:837-855(1997); and Moreau, P., et al., J. Med. Chem. 41:1631-1640(1998)), antisense oligonucleotides (e.g., those disclosed in the U.S. Pat. Nos. 6,277,832, 5,998,596, 5,885,834, 5,734,033, and 5,618,709), immunomodulators (e.g., antibodies and cytokines), antibodies, and adenosine deaminase inhibitors (e.g., Fludarabine phosphate and 2-Chlorodeoxyadenosine).

Further, an antibody or antibody fragment may be conjugated to a therapeutic moiety or drug moiety that modifies a given biological response. Therapeutic or drug moieties are not to be construed as limited to classical chemical therapeutic agents. For example, the drug moiety may be a protein, polypeptide or peptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, γ-interferon, nerve growth factor, platelet derived growth factor, tissue plasminogen activator, an apoptotic agent, e.g., TNF-α, TNF-β, AIM I (see, International publication No. WO 97/33899), AIM II (see, International Publication No. WO 97/34911), Fas Ligand (Takahashi et al., 1994, J. Immunol., 6:1567-1574), and VEGF (see International publication No. WO 99/23105); or, a biological response modifier such as, for example, a lymphokine (e.g., interleukin-1 (“IL-1”), interleukin-2 (“IL-2”), interleukin-4 (“IL-4”), interleukin-6 (“IL-6”), interleukin-9 (“IL-9”), interleukin-10 (“IL-10”), interleukin-12 (“IL-12”), granulocyte macrophage colony stimulating factor (“GM-CSF”), and granulocyte colony stimulating factor (“G-CSF”)), and a growth factor (e.g., growth hormone (“GH”)).

Moreover, an antibody or antibody fragment can be conjugated to therapeutic moieties such as a radioactive metal ion, such as alph-emiters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody can be conjugated to a second antibody to form an antibody heteroconjugate as described by Segal in U.S. Pat. No. 4,676,980, which is incorporated herein by reference in its entirety.

The therapeutic moiety, drug or antibody conjugated to an antibody or antibody fragment that immunospecifically binds to integrin αᵥβ₃should be chosen to achieve the desired prophylactic or therapeutic effect(s) for a particular disorder in a subject. A clinician or other medical personnel should consider the following when deciding on which therapeutic moiety or drug to conjugate to an antibody or antibody fragment that immunospecifically binds to integrin αᵥβ₃ the nature of the disease, the severity of the disease, and the condition of the subject.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Therapeutic moieties or drugs such as those described herein can be conjugated to integrin αᵥβ₃ antagonists other than antibodies using techniques well-known in the art.

4.2 Methods of Identifying Antibodies Immunospecific for Integrin αᵥβ₃

The invention provides methods for identifying antagonists that are immunospecific for integrin αᵥβ₃, particularly for antibodies that specifically bind to the same epitope as VITAXIN® and/or LM609. Mutation of residues 171, 173 and/or 174 of the human 03 chain have been found to disrupt binding of VITAXIN® and/or LM609 antibodies to the integrin αᵥβ₃ heterodimer. Although VITAXIN® and LM609 do not bind to mouse integrin αᵥβ₃, it has been found that VITAXIN® and LM609 do bind to a modified mouse integrin αᵥβ₃ in which the region of the mouse 0 chain that corresponds to amino acids 164-202 of the human β chain are replaced with amino acids 164-202 of the human 0 chain. In certain embodiments, amino acid substitutions are made in the subunits of integrin αᵥβ₃, for example to change the ligand specificity of the integrin αᵥβ₃ and/or disrupt the heterodimerization of the subunit chains. Preferably the integrin αᵥβ₃ is human. In specific embodiments, such amino acid substitutions disrupt the specific interaction of certain antagonists of integrin αᵥβ₃ with a particular integrin αᵥβ₃ epitope. In a preferred embodiment, the amino acid substitutions are made within regions of an integrin subunit that confers ligand binding specificity, preferably ligand binding specificity of LM609 and/or VITAXIN®, particularly residues 164-202 of human β₃. Alternatively, mouse β chain residues corresponding to residues 164-202 of the human β₃ chain are replaced with the residues 164-202 of the human β₃ chain. Such mouse-human chimeras can be used to screen for antagonists that bind to the region 164-202 of human β₃ but not to mouse integrin αᵥβ₃.

In preferred embodiments, the amino acid substitutions are made in the β₃subunit. In certain embodiments, human β₃ residues are substituted with rat residues as described in Table 2. In one embodiment, the substitution of human residue Glu to rat residue Gln at position 171 (“Mutation A”) disrupts integrin αᵥβ₃ binding to LM609. This same change disrupts binding to VITAXIN®. In another embodiment, the substitution of human residue Leu and Glu to rat residues Ile, and Lys at positions 173 and 174, respectively (“Mutation B”) both disrupt binding to VITAXIN® and increase binding to an anti rat 3 antibody. In yet another embodiment, the substitution of human residues Asp and Thr to rat residues Thr and Ser at positions 179 and 182 respectively (“Mutation C”) confer binding specificity to an anti-rat β₃ antibody. Mutations A and C combined (three substituted residues) confer binding specificity for the mouse-anti-rat β₃ antibody and disrupts binding to VITAXIN®. In a specific preferred embodiment, amino acids 171, 173 and 174 can be substituted to disrupt binding to VITAXIN®. In an alternate preferred embodiment, amino acids 171, 173, 174, 179 and 182 can be substituted to disrupt binding of integrin αᵥβ₃ to LM609 and humanized anti-integrin αᵥβ₃ antibodies such as VITAXIN®. Such substitutions preferred examples but not limiting. Such substituted subunits are merely exemplary and not limiting. Any integrin αᵥβ₃ regions identified to be responsible for antibody binding can be altered with substituted, deleted or inserted residues to characterize binding specificity of various antibodies and to screen for antibodies with the same a similar binding specificity.

Amino acid substituted subunits of integrin αᵥβ₃ can be used for screening antibodies with specific affinity for particular epitopes by identifying monoclonal antibodies that bind to wild type integrin αᵥβ₃ but not the altered form, or that bind mouse αᵥβ₃ integrins with a region substituted with the corresponding region from the human otv3 but do not bind to wild type mouse integrin αᵥβ₃. In addition, the invention provides methods for identifying monoclonal antibodies that bind to the heterodimerized αᵥβ₃ but not the αᵥ or the β₃ chains when not included in a heterodimer. Such screening can be accomplished by any routine method for assaying antibody specificity known in the art, for example, using cell lines that do not express wild type integrin αᵥβ₃ to recombinantly express the mutant αᵥβ₃ or individual αᵥ or β₃ chains. The antibodies identified from such screening methods can be useful for the prevention, treatment, management and amelioration of integrin αᵥβ₃-mediated diseases and disorders, including but not limited to periodontal disease, Gorham-Stout disease, Wilson's disease, chronic otitis media, hypertrophic pulmonary osteoarthropathy or aseptic loosening of joint replacements or a condition or symptom associated therewith. It is also contemplated that such antibodies can be used in the methods and compositions contemplated by the present invention. Preferably, these antibodies are not LM609, VITAXIN® or an antibody or antigen-binding fragment thereof having the CDRs (or one, two, three, four or five of the CDRs or CDR3 of the heavy chain) of LM609 or VITAXIN® with no more than one, no more than two, no more than five, no more than eight, or no more than ten amino acid substitutions, deletions or insertions.

TABLE 2

| Human Beta3 | Mutation A | Mutation B | | Mutation C | |
|---|---|---|---|---|---|
| mutants | (Glu-Gln) | (Leu-Ile),(Glu-Lys) | | (Asp-Thr),(Thr-Ser) | |
| A1(A, C) | E171Q | | | D179T | T182S |
| A6 | E171Q | | | | |
| B1 | | L173 I | E174K | | |
| C14 | | | | D179T | T182S |
| C16 | | | | D179T | T182S |
| ABC17 | E171Q | L173 I | E174K | D179T | T182S |

4.3 Other Agents Used in Combination with Integrin αᵥβ₃ Antagonists

The present invention provides compositions comprising an integrin αᵥβ₃ antagonist (e.g., an antibody that immunospecifically binds to integrin αᵥβ₃) and/or at least one prophylactic or therapeutic agent other than an antibody that immunospecifically binds to integrin αᵥβ₃, and methods for preventing, treating, managing or ameliorating periodontal disease, Gorham-Stout disease, Wilson's disease, chronic otitis media, hypertrophic pulmonary osteoarthropathy or aseptic loosening of joint replacement or a condition or symptom associated therewith, comprising administering to a subject in need thereof one or more of said compositions. Therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. Any agent which is known to be useful, or which has been used or is currently being used for the prevention, treatment or amelioration of a periodontal disease, Gorham-Stout disease, Wilson's disease, chronic otitis media, hypertrophic pulmonary osteoarthropathy or aseptic loosening of joint replacement or a condition or symptom associated therewith can be used in combination with an antibody that immunospecifically binds to integrin αᵥβ₃ in accordance with the invention described herein. See, e.g., the Physician's Desk Reference (57^{th} ed., 2003) for information regarding prophylactic or therapeutic agents which have been or are currently being used for treating, preventing, managing, or ameliorating periodontal disease, Gorham-Stout disease, Wilson's disease, chronic otitis media, hypertrophic pulmonary osteoarthropathy or aseptic loosening of joint replacement or a condition associated therewith. Examples of such agents include, but are not limited to, analgesics, anti-inflammatory agents, dental preparations, anti-arthritic agents, inhibitors of metalloproteinases, antibiotics (including Periostat™), chelating agents, zinc acetate (Galzing), bone metabolism regulating agents, hormones, vitamins and immunomodulatory agents.

4.3.1 Analgesics

Any analgesics well-known to one of skill in the art can be used in the compositions and the methods of the invention. Non-limiting examples of analgesics which can be used in accordance with the invention include: NSAIDs, salicylates, acetominophen, narcotics, and non-narcotic and anxiolytic combinations.

Analgesic agents and therapies and their dosages, routes of administration and recommended usage are known in the art and have been described in such literature as the Physician's Desk Reference (57^{th} ed., 2003).

4.3.2 Anti-Inflammatory Agents

Any anti-inflammatory agents well-known to one of skill in the art can be used in the compositions and the methods of the invention. Non-limiting examples of anti-inflammatory agents which can be used in accordance with the invention to include: non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFONm), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

Anti-inflammatory agents and therapies and their dosages, routes of administration and recommended usage are known in the art and have been described in such literature as the Physician's Desk Reference (57 h ed., 2003).

4.3.3 Dental Preparations

Any dental preparations well-known to one of skill in the art can be used in the compositions and the methods of the invention. Non-limiting examples of dental preparations that which can be used in accordance with the invention include fluoride, calcium, rinses (e.g., LISTERINE™), antibiotics (e.g., doxycycline hyclate a.k.a. PERIOSTAT™ (CollaGenex Pharmaceuticals, Inc., Newtown, Pa.), and Atridox™ (Atrix Laboratories, Inc.), fluoride and calcium supplements (e.g., FLORICAL™ (Mericon Industries, Inc., Peoria, Ill.) and MONOCAL™ (Mericon Industries, Inc., Peoria, Ill.)).

In addition, periodontal vaccines can also be used in accordance with the invention. See DeCarlo, A., et al., 2003, Infect. Immun. 71(1):562-6 (perinoculation with Porphyromonas gingival HA2 binding domain for hemoglobin found to provide protection from periodontitis in rat periodontitis model); and Rajapakse, P. et al., 2002, Infection and Immunity 70(5):2480-2486 (immunization with RgpA-Kgp proteinase-adhesion complexes of Porphyromonas gingivalis found to protect against periodontal bone loss in rat periodontitis model), incorporated by reference in their entireties.

Dental preparations and therapies and their dosages, routes of administration and recommended usage are known in the art and have been described in such literature as the Physician's Desk Reference (57^{th} ed., 2003).

4.3.4 Anti-Arthritics

Any anti-arthritics well-known to one of skill in the art can be used in the compositions and the methods of the invention. Non-limiting examples of anti-arthritics which can be used in accordance with the invention include: analgesics (non-limiting examples are acetaminophen, in a dose up to 4000 mg/d; phenacetin; and tramadol, in a daily dose in the range of 200 to 300 mg); NSAIDs (non-limiting examples include but not limited to, aspirin, diflunisal, diclofenac, etodolac, fenamates, fenoprofen, flurbiprofen, ibuprofen, indomethacin, ketoprofen, methylsalicylate, nebumetone, naproxin, oxaprazin, phenylbutazone, piroxicam, sulindac, and tolmetin); nonacetylated salicylates such as salsalate; cyclooxygenase (Cox)-2-specific inhibitors (CSIs), such as, celecoxib and rofecoxib; intra- or periarticular injection of a depot glucocorticoid preparation; intra-articular injection of hyaluronic acid; capsaicin cream; copious irrigation of the osteroarthritis knee to flush out fibrin, cartilage shards and other debris; and joint replacement surgery. Low dose NSAIDs are preferred, e.g., ibuprofen at 1200 mg/d, naproxen at 500 mg/d. A gastroprotective agent, e.g., misoprostol, famotidine or omeprazole, is preferred to use concurrently with a NSAID.

The compositions of the invention can also be used in combination with other nonpharmacologic measures including but not limited to: reduction of joint loading (non-limiting examples are correction of poor posture, support for excessive lumbar lordosis, avoid excessive loading of the involved joint, avoid prolonged standing, kneeling and squatting); application of heat to the affected joint; aerobic exercise and other physical therapies.

Anti-arthritic agents and therapies and their dosages, routes of administration and recommended usage are known in the art and have been described in such literature as the Physician's Desk Reference (57^{th} ed., 2003).

4.3.5 Inhibitors of Metalloproteinases

Any inhibitors of metalloproteinases well-known to one of skill in the art can be used in the compositions and the methods of the invention. Non-limiting examples of inhibitors of metalloproteinases include: Marimastat, BB94 (batimastat; [4-(N-hydroxyamino)-2R-isobutyl-3 S-thienyl-thiomethyl)-succinyl-]L-phenylalanine-N-methylamide; British Pharmaceuticals Limited (Oxford, UK)), Streptomyces metalloproteinase inhibitor (SMPI), BB-3103, and tissue inhibitors of metalloproteinases (e.g., TIMP-1, TIMP-2 and TIMP-3).

Inhibitors of metalloproteinases and therapies and their dosages, routes of administration and recommended usage are known in the art and have been described in such literature as the Physician's Desk Reference (57^{th} ed., 2003).

4.3.6 Antibiotics

Any antibiotics well-known to one of skill in the art can be used in the compositions and the methods of the invention. Non-limiting examples of antibiotics include, penicillin, doxycycline, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

Antibiotic agents and therapies and their dosages, routes of administration and recommended usage are known in the art and have been described in such literature as the Physician's Desk Reference (57^{th} ed., 2003).

4.3.7 Bone Metabolism Regulating Agents

Bone metabolism regulating agents include, but are not limited to, peptides, polypeptides, proteins, fusion proteins, nucleic acid molecules, small molecules, mimetic agents, synthetic drugs, inorganic molecules, and organic molecules. Any agent or therapy which is known to be useful, or which has been used or is currently being used to regulate bone metabolism can be used in combination with an integrin αᵥβ₃ antagonist in accordance with the invention described herein. Non-limiting examples of bone metabolism regulating agents include phosphate, aluminum hydroxide, aluminum carbonate gels, magnesium, vitamin D, calcitriol, vitamin D₂ (ergocalciferol), vitamin D₃ (cholecalciferol), calcium, lithium, glucocorticoids, biphosphonates or a pharmaceutically acceptable salt or ester thereof (non-limiting examples are alendronate, clodronate, etidronate, ibandronate, pamidronate, risedronate, tiludronate, and zoledronate), calcitonin, plicamycin (mithramycin), gallium nitrate, estrogens, progestins, estrogen antagonists (e.g., tamoxifen), estrogen receptor modulators, androgen receptor modulators, cytotoxic or antiproliferative agents, matrix metalloproteinase inhibitors, inhibitors of epidermal-derived, fibroblast-derived, or platelet-derived growth factors, inhibitors of VEGF, antibodies to a growth factor or to a growth factor receptor, inhibitors of Flk-1/KDR, Flt-1, Tck/Tie-2, or Tie-1, cathepsin K inhibitors, inhibitors of osteoclast proton ATPase, inhibitors of urokinase plasminogen activator (u-PA), tumor-specific antibody-interleukin-2 fusion proteins, inhibitors of HMG-CoA reductase (non-limiting examples are Lovastatin, Pravastatin, Fluvastatin, Statin, Simvastatin, cerivastatin, lescol, lupitor, rosuvastatin and Atorvastatin), prenylation inhibitors, farnesyl transferase inhibitors, geranylgeranyl transferase inhibitors or dual farnesyl/geranylgeranyl transferase inhibitors, parathyroid hormone or parathyroid hormone fragments (a non-limiting example is exogenous PTH analogue, 1-34 PTH), growth hormones, molecules disclosed in U.S. Pat. Nos. 6,472,402 and 6,482,411, or a combination thereof. Non-limiting examples of bone metabolism regulating therapies include renal dialysis and surgery.

Bone metabolism regulating agents and therapies and their dosages, routes of administration and recommended usage are known in the are and have been described in such literatures as the Physician's Desk Reference (57^{th} ed., 2003).

4.3.8 Hormones

Any hormone well-known to one of skill in the art can be used in the compositions and the methods of the invention. Non-limiting examples of hormones which can be used in the compositions and methods of the invention include calcitonin, glucocorticoids, and estrogen.

Homones and therapies and their dosages, routes of administration and recommended usage are known in the are and have been described in such literatures as the Physician's Desk Reference (57^{th} ed., 2003).

4.3.9 Immunomodulatory Agents

Any immunomodulatory agent well-known to one of skill in the art can be used in the compositions and the methods of the invention. Non-limiting examples of immunomodulatory agents which can be used in used in accordance with the invention include chemotherapeutic agents and immunomodulatory agents other than chemotherapeutic agents. Examples of chemotherapeutic agents include, but are not limited to: methotrexate, cyclosporin A, malononitriloamindes (e.g., leflunamide), cisplatin, ifosfamide, taxanes such as taxol and paclitaxol, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, Immuran, minocycline, azathioprine, antibiotics (e.g., FK506 (tacrolimus)), methylprednisolone (MP), corticosteroids, steriods, mycophenolate mofetil, rapamycin (sirolimus), mizoribine, deoxyspergualin, brequinar, glucocorticoids, procaine, tetracaine, lidocaine, propranolol, and puromycin homologs, and cytoxan. Examples of immunomodulatory agents other than chemotherapeutic agents include, but are not limited to: anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., MEDI-507 (MedImmune, Inc., International Publication Nos. WO 02/098370 and WO 02/069904), anti-CD11a antibodies (e.g., Xanelim (Genentech)), and anti-B7 antibodies (e.g., IDEC-114) (IDEC)); anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies); CTLA4-immunoglobulin; LFA-3TIP (Biogen, International Publication No. WO 93/08656 and U.S. Pat. No. 6,162,432); soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof); cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF); and anti-cytokine antibodies (e.g., anti-IFN antibodies, anti-IFN-γ antibodies, anti-TNF-α antibodies, anti-IL-1β antibodies, anti-IL-2 antibodies, anti-IL-4 antibodies, anti-IL-6 antibodies, anti-IL-8 antibodies (e.g., ABX-IL-8 (Abgenix)), anti-IL-9 antibodies, anti-IL-10 antibodies, anti-IL-12 antibodies, anti-IL-15 antibodies).

In a specific embodiment, the immunomodulatory agent is not a chemotherapeutic agent. In another embodiment, the immunomodulatory agents is a chemotherapeutic agent.

Immunomodulatory agents and therapies and their dosages, routes of administration and recommended usage are known in the are and have been described in such literatures as the Physician's Desk Reference (57^{th} ed., 2003).

4.3.10 Chelating Agents

Any chelating agent well-known to one of skill in the art can be used in the compositions and the methods of the invention. Non-limiting examples of chelating agents include D-pencillamine (Cuprimine®, and Depen®), trientine (Syprine), and tetrathiomolybdate.

Chelating agents and their dosages, routes of administration and recommended usage are known in the are and have been described in such literatures as the Physician's Desk Reference (57^{th} ed., 2003).

4.3.11 Vitamins and Minerals

Any vitamin and/or mineral well-known to one of skill in the art can be used in the compositions and the methods of the invention. Non-limiting examples of vitamins and minerals include magnesium, calicum, vitamin C compounds, vitamin B compounds such as vitamin B6 (pyridoxine) and vitamin D compounds. Preferably, a multivitamin and a mineral supplement is used in accordance with the invention.

Vitamins and minerals and their dosages, routes of administration and recommended usage are known in the are and have been described in such literatures as the Physician's Desk Reference (57th ed., 2003).

4.3.12 Anti-Viral Agents

Any anti-viral agent well-known to one of skill in the art can be used in the compositions and the methods of the invention. Non-limiting examples of anti-viral agents include proteins, polypeptides, peptides, fusion protein antibodies, nucleic acid molecues, organic molecules, inorganic molecules, and small molecules that inhibit or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, alpha-interferons, gamma-interferons and other interferons, and AZT.

In specific embodiments, the anti-viral agent is an immunomodulatory agent that is immunospecific for a viral antigen. As used herein, the term “viral antigen” includes, but is not limited to, any viral peptide, polypeptide and protein (e.g., RSV F glycoprotein, RSV G glycoprotein, influenza virus neuraminidase, an influenza virus hemagglutinin) that is capable of eliciting an immune response. Antibodies useful in this invention for treatment of a viral infectious disease include, but are not limited to, antibodies against antigens of pathogenic viruses, including as examples and not by limitation: adenovirdiae (e.g., mastadenovirus and aviadenovirus), paramyxoviridae (e.g., paramyxovirus, parainfluenza virus 1, pneumonovirinae (e.g., pneumovirus, human respiratory synctial virus), and metapneumovirus (e.g., avian pneumovirus and human metapneumovirus)), picornaviridae and (e.g., rhinovirus). Specific examples of antibodies available useful for the prevention or treatment of a viral infectious disease include, but are not limited to, SYNAGIS® (MedImmune, Inc.; International Publication No. WO 02/43660) which is a humanized antibody useful for treatment of RSV.

Anti-viral agents and therapies and their dosages, routes of administration and recommended usage are known in the are and have been described in such literatures as the Physician's Desk Reference (57^{th} ed., 2003).

4.4 Prophylactic and Therapeutic Uses

4.4.1 Treatment of Periodontal Disease

The present invention provides methods for preventing, managing, treating or ameliorating periodontal disease or a symptom thereof, said methods comprising administering to a subject in need thereof an integrin αᵥβ₃ antagonist. The present invention also provides methods for preventing, managing, treating or ameliorating periodontal disease or a symptom thereof, said methods comprising administering to a subject in need thereof an integrin αᵥβ₃ antagonist and at least one therapy other than an integrin αᵥβ₃ antagonist (e.g., a prophylactic or therapeutic agent such as those disclosed in Section 4.3 supra). In a specific embodiment, the invention provides methods for preventing, managing, treating or ameliorating periodontal disease or a symptom thereof, said methods comprising administering to a subject in need thereof a dose of a prophylactically or therapeutically effective amount of an integrin αᵥβ₃ antagonist and a dose of a prophylactically or therapeutically effective amount of at least one of the following therapies: an antibiotic, surgery, an anti-inflammatory agent, an immunomodulatory agent, an anti-anthritic, hormonal therapy, CO₂ laser therapy, root planing and/or a bone metabolism regulating agent. Preferably, the integrin αᵥβ₃ antagonist is an antibody or antibody fragment that immunospecifically binds to integrin αᵥβ₃—In a specific embodiment, the antibody is VITAXIN™.

In one embodiment, the invention provides a method for preventing, managing, treating or ameliorating periodontal disease or a symptom thereof, said methods comprising administering to a subject in need thereof a dose of a prophylactically or therapeutically effective amount of an integrin αᵥβ₃ antagonist and a dose of a prophylactically or therapeutically effective amount of Periostat™. In another embodiment, the invention provides a method for preventing, managing, treating or ameliorating periodontal disease or a symptom thereof, said methods comprising administering to a subject in need thereof a dose of a prophylactically or therapeutically effective amount of an integrin αᵥβ₃ antagonist and a dose of a prophylactically or therapeutically effective amount of a bisphosphonate. In another embodiment, the invention provides a method for preventing, managing, treating or ameliorating periodontal disease or a symptom thereof, said methods comprising administering to a subject in need thereof a dose of a prophylactically or therapeutically effective amount of an integrin αᵥβ₃ antagonist and a dose of a prophylactically or therapeutically effective amount of an anti-inflammatory agent (e.g., an NSAID). In another embodiment, the invention provides a method for preventing, managing, treating or ameliorating periodontal disease or a symptom thereof, said methods comprising administering to a subject in need thereof a dose of a prophylactically or therapeutically effective amount of an integrin αᵥβ₃ antagonist and a dose of a prophylactically or therapeutically effective amount of anti-Porphyromonas gingival HA2 binding domain IgG.

The compositions and methods of the invention may be used as first, second, third or fourth line of treatment for a periodontal disease. The invention provides methods for managing, treating or ameliorating a periodontal disease or a symptom thereof in a subject refractory to conventional therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides methods for managing, treating or ameliorating a periodontal disease or a symptom thereof in a subject refractory to existing single agent therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides alternative methods for the management or treatment of a periodontal disease where conventional therapies have proven or may prove too toxic, i.e., results in unacceptable or unbearable side effects, for the subject being treated. Further, the invention provides methods for preventing the recurrence of a periodontal disease in patients that have been treated and have no disease activity by administering a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra).

In each of the foregoing embodiments, a nucleic acid encoding an integrin αᵥβ₃ antagonist may be administered instead of, or in combination with, an integrin αᵥβ₃ antagonist.

Periodontal diseases include, but are not limited to, gingivitis, periodontitis, and periodonosis.

4.4.2 Treatment of Aseptic Loosening of a Joint Replacement

The methods for preventing, managing, treating or ameliorating aseptic loosening of a joint replacement or a condition or symptom associated therewith, said methods comprising administering to a subject in need thereof an integrin αᵥβ₃ antagonist. The present invention also provides methods for preventing, managing, treating or ameliorating aseptic loosening of a joint replacement or a condition or symptom associated therewith, said methods comprising administering to a subject in need thereof an integrin αᵥβ₃ antagonist and a therapy other than integrin αᵥβ₃ antagonists (e.g., a prophylactic or therapeutic agent such as those disclosed in Section 4.3 supra). Non-limiting examples of conditions associated with aseptic loosening of a joint replacement include inflammation and arthritis.

In a specific embodiment, the invention provides methods for preventing, managing, treating or ameliorating aseptic loosening of a joint replacement or a condition or symptom associated therewith, said methods comprising administering to a subject in need thereof a dose of a prophylactically or therapeutically effective amount of an integrin αᵥβ₃ antagonist and a dose of a prophylactically or therapeutically effective amount of at least one of the following therapies: an antibiotic, surgery, an anti-inflammatory agent, an immunomodulatory agent, an anti-arthritic, hormonal therapy and/or a bone metabolism regulating agent. Preferably, the integrin αᵥβ₃ antagonist is an antibody or antibody fragment that immunospecifically binds to integrin αᵥβ₃. In one embodiment, the antibody is VITAXIN™.

In one embodiment, the invention provides a method for preventing, managing, treating or ameliorating aseptic loosening of a joint replacement or a condition or symptom associated therewith, said methods comprising administering to a subject in need thereof a dose of a prophylactically or therapeutically effective amount of an integrin αᵥβ₃ antagonist and a dose of a prophylactically or therapeutically effective amount of a bisphosphonate. In another embodiment, the invention provides a method for preventing, managing, treating or ameliorating aseptic loosening of a joint replacement or a condition or symptom associated therewith, said methods comprising administering to a subject in need thereof a dose of a prophylactically or therapeutically effective amount of an integrin αᵥβ₃ antagonist and a dose of a prophylactically or therapeutically effective amount of an anti-inflammatory agent (e.g., an NSAID). In another embodiment, the invention provides a method for preventing, managing, treating or ameliorating aseptic loosening of a joint replacement or a condition or symptom associated therewith, said methods comprising administering to a subject in need thereof a dose of a prophylactically or therapeutically effective amount of an integrin αᵥβ₃ antagonist and a dose of a prophylactically or therapeutically effective amount of anti-arthritic agents. In another embodiment, the invention provides a method for preventing, managing, treating or ameliorating aseptic loosening of a joint replacement or a condition or symptom associated therewith, said methods comprising administering to a subject in need thereof a dose of a prophylactically or therapeutically effective amount of an integrin αᵥβ₃ antagonist and a dose of a prophylactically or therapeutically effective amount of inhibitors of metalloproteinases.

The compositions and methods of the invention may be used as first, second, third or fourth line of treatment for aseptic loosening of a joint replacement. The invention provides methods for managing, treating or ameliorating aseptic loosening of a joint replacement or conditions associated therewith, in a subject refractory to conventional therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides methods for managing, treating or ameliorating aseptic loosening of a joint replacement or a symptom thereof in a subject refractory to existing single agent therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides alternative methods for the management or treatment of aseptic loosening of a joint replacement where conventional therapies have proven or may prove too toxic, i.e., results in unacceptable or unbearable side effects, for the subject being treated. Further, the invention provides methods for preventing the recurrence of aseptic loosening of a joint replacement in patients that have been treated and have no disease activity by administering a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra).

In each of the foregoing embodiments, a nucleic acid encoding an integrin αᵥβ₃ antagonist may be administered instead of, or in combination with, an integrin αᵥβ₃ antagonist.

In a specific embodiment, the aseptic loosening of a joint is the aseptic loosening of a hip or knee.

4.4.3 Treatment of Wilson's Disease

The methods for preventing, managing, treating or ameliorating Wilson's disease or a symptom thereof, said methods comprising administering to a subject in need thereof an integrin αᵥβ₃ antagonist. The present invention also provides methods for preventing, managing, treating or ameliorating Wilson's disease or a symptom thereof, said methods comprising administering to a subject in need thereof an integrin αᵥβ₃ antagonist and at least one therapy other than an integrin αᵥβ₃ antagonist (e.g., at least one other prophylactic or therapeutic agent such as those disclosed in Section 4.3 supra).

In a specific embodiment, the invention provides methods for preventing, managing, treating or ameliorating Wilson's disease or a symptom thereof, said methods comprising administering to a subject in need thereof a dose of a prophylactically or therapeutically effective amount of an integrin αᵥβ₃ antagonist and a dose of a prophylactically or therapeutically effective amount of at least one of the following therapies: an anti-inflammatory agent, an immunomodulatory agent, chelating agents, and/or zinc acetate. Preferably, the integrin αᵥβ₃ antagonist is an antibody or antibody fragment that immunospecifically binds to integrin αᵥβ₃. In one embodiment, the antibody is VITAXIN™.

In one embodiment, the invention provides a method for preventing, managing, treating or ameliorating Wilson's disease or a symptom thereof, said methods comprising administering to a subject in need thereof a dose of a prophylactically or therapeutically effective amount of an integrin αᵥβ₃ antagonist and a dose of a prophylactically or therapeutically effective amount of a chelating agent. In another embodiment, the invention provides a method for preventing, managing, treating or ameliorating Wilson's disease or a symptom thereof, said methods comprising administering to a subject in need thereof a dose of a prophylactically or therapeutically effective amount of an integrin αᵥβ₃ antagonist, a dose of a prophylactically or therapeutically effective amount of D-penicillamine and a dose of a prophylactically or therapeutically effective amount of pyridoxine. In another embodiment, the invention provides a method for preventing, managing, treating or ameliorating Wilson's disease or a symptom thereof, said methods comprising administering to a subject in need thereof a dose of a prophylactically or therapeutically effective amount of an integrin αᵥβ₃ antagonist and a dose of a prophylactically or therapeutically effective amount of an anti-inflammatory agent (e.g., an NSAID). In another embodiment, the invention provides a method for preventing, managing, treating or ameliorating Wilson's disease or a symptom thereof, said methods comprising administering to a subject in need thereof a dose of a prophylactically or therapeutically effective amount of an integrin αᵥβ₃ antagonist and a dose of a prophylactically or therapeutically effective amount of zinc acetate. In another embodiment, the invention provides a method for preventing, managing, treating or ameliorating Wilson's disease or a symptom thereof, said methods comprising administering to a subject in need thereof a dose of a prophylactically or therapeutically effective amount of an integrin αᵥβ₃ antagonist, a dose of a prophylactically or therapeutically effective amount of D-penicillamine, a dose of a prophylactically or therapeutically effective amount of pyridoxine a dose of a prophylactically or therapeutically effective amount of zinc acetate.

The compositions and methods of the invention may be used as first, second, third or fourth line of treatment for Wilson's disease or a symptom thereof. The invention provides methods for managing, treating or ameliorating Wilson's disease or a symptom thereof, in a subject refractory to conventional therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides methods for managing, treating or ameliorating Wilson's disease or a symptom thereof in a subject refractory to existing single agent therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides alternative methods for the management or treatment of Wilson's disease or a symptom thereof where conventional therapies have proven or may prove too toxic, i.e., results in unacceptable or unbearable side effects, for the subject being treated. Further, the invention provides methods for preventing the recurrence of Wilson's disease or a symptom thereof in patients that have been treated and have no disease activity by administering a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra).

In each of the foregoing embodiments, a nucleic acid encoding an integrin αᵥβ₃ antagonist may be administered instead of, or in combination with, an integrin αᵥβ₃ antagonist.

4.4.4 Treatment of Gorham-Stout Disease

The methods for preventing, managing, treating or ameliorating Gorham-Stout disease or a symptom thereof, said methods comprising administering to a subject in need thereof an integrin αᵥβ₃ antagonist. The present invention also provides methods for preventing, managing, treating or ameliorating Gorham-Stout disease or a symptom thereof, said methods comprising administering to a subject in need thereof an integrin αᵥβ₃ antagonist and at least one therapy other than integrin αᵥβ₃ antagonists (e.g., a prophylactic or therapeutic agent such as those disclosed in Section 4.3 supra).

In a specific embodiment, the invention provides methods for preventing, managing, treating or ameliorating Gorham-Stout disease or a symptom thereof, said methods comprising administering to a subject in need thereof a dose of a prophylactically or therapeutically effective amount of an integrin αᵥβ₃ antagonist and a dose of a prophylactically or therapeutically effective amount of at least one of the following therapies: an anti-inflammatory agent, an immunomodulatory agent, a bone metabolism regulating agent, an inhibitor of metalloproteinases, and/or hormonal therapy. Preferably, the integrin αᵥβ₃ antagonist is an antibody or antibody fragment that immunospecifically binds to integrin αᵥβ₃. In one embodiment, the antibody is VITAXIN™.

In one embodiment, the invention provides a method for preventing, managing, treating or ameliorating Gorham-Stout disease or a symptom thereof, said methods comprising administering to a subject in need thereof a dose of a prophylactically or therapeutically effective amount of an integrin αᵥβ₃ antagonist and a dose of a prophylactically or therapeutically effective amount of an anti-inflammatory agent (e.g., an NSAID). In another embodiment, the invention provides a method for preventing, managing, treating or ameliorating Gorham-Stout disease or a symptom thereof, said methods comprising administering to a subject in need thereof a dose of a prophylactically or therapeutically effective amount of an integrin αᵥβ₃ antagonist and a dose of a prophylactically or therapeutically effective amount of an inhibitor of a metalloproteinase. In another embodiment, the invention provides a method for preventing, managing, treating or ameliorating Gorham-Stout disease or a symptom thereof, said methods comprising administering to a subject in need thereof a dose of a prophylactically or therapeutically effective amount of an integrin αᵥβ₃ antagonist and a dose of a prophylactically or therapeutically effective amount of an anti-IL-6 antibody.

The compositions and methods of the invention may be used as first, second, third or fourth line of treatment for Gorham-Stout disease or a symptom thereof. The invention provides methods for managing, treating or ameliorating Gorham-Stout disease or a symptom thereof, in a subject refractory to conventional therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides methods for managing, treating or ameliorating Gorham-Stout disease or a symptom thereof in a subject refractory to existing single agent therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides alternative methods for the management or treatment of Gorham-Stout disease or a symptom thereof where conventional therapies have proven or may prove too toxic, i.e., results in unacceptable or unbearable side effects, for the subject being treated. Further, the invention provides methods for preventing the recurrence of Gorham-Stout disease or a symptom thereof in patients that have been treated and have no disease activity by administering a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra).

In each of the foregoing embodiments, a nucleic acid encoding an integrin αᵥβ₃ antagonist may be administered instead of, or in combination with, an integrin αᵥβ₃ antagonist.

4.4.5 Treatment of Chronic Otitis Media

The methods for preventing, managing, treating or ameliorating chronic otitis media or a symptom thereof, said methods comprising administering to a subject in need thereof an integrin αᵥβ₃ antagonist. The present invention also provides methods for preventing, managing, treating or ameliorating chronic otitis media or a symptom thereof, said methods comprising administering to a subject in need thereof an integrin αᵥβ₃ antagonist and at least one therapy other than integrin αᵥβ₃ antagonists (e.g., a prophylactic or therapeutic agent such as those disclosed in Section 4.3 supra).

In a specific embodiment, the invention provides methods for preventing, managing, treating or ameliorating chronic otitis media or a symptom thereof, said methods comprising administering to a subject in need thereof a dose of a prophylactically or therapeutically effective amount of an integrin αᵥβ₃ antagonist and a dose of a prophylactically or therapeutically effective amount of at least one of the following therapies: an anti-inflammatory agent, an immunomodulatory agent, a bone metabolism regulating agent, an antibiotic, an anti-viral agent and/or surgery. Preferably, the integrin αᵥβ₃ antagonist is an antibody or antibody fragment that immunospecifically binds to integrin αᵥβ₃. In one embodiment, the antibody is VITAXIN™.

In one embodiment, the invention provides a method for preventing, managing, treating or ameliorating chronic otitis media or a symptom thereof, said methods comprising administering to a subject in need thereof a dose of a prophylactically or therapeutically effective amount of an integrin αᵥβ₃ antagonist and a dose of a prophylactically or therapeutically effective amount of an antibiotic. In another embodiment, the invention provides a method for preventing, managing, treating or ameliorating chronic otitis media or a symptom thereof, said methods comprising administering to a subject in need thereof a dose of a prophylactically or therapeutically effective amount of an integrin αᵥβ₃ antagonist and a dose of a prophylactically or therapeutically effective amount of a bone metabolism regulating agent. In another embodiment, the invention provides a method for preventing, managing, treating or ameliorating chronic otitis media or a symptom thereof, said methods comprising administering to a subject in need thereof a dose of a prophylactically or therapeutically effective amount of an integrin αᵥβ₃ antagonist and a dose of a prophylactically or therapeutically effective amount of an anti-inflammatory agent (e.g., an NSAID). In another embodiment, the invention provides a method for preventing, managing, treating or ameliorating chronic otitis media or a symptom thereof, said methods comprising administering to a subject in need thereof a dose of a prophylactically or therapeutically effective amount of an integrin αᵥβ₃ antagonist and a dose of a prophylactically or therapeutically effective amount of an antibiotic. In another embodiment, the invention provides a method for preventing, managing, treating or ameliorating chronic otitis media or a symptom thereof, said methods comprising administering to a subject in need thereof a dose of a prophylactically or therapeutically effective amount of an integrin αᵥβ₃ antagonist and a dose of a prophylactically or therapeutically effective amount of an immunomodulatory agent.

The compositions and methods of the invention may be used as first, second, third or fourth line of treatment for chronic otitis media or a symptom thereof. The invention provides methods for managing, treating or ameliorating chronic otitis media or a symptom thereof, in a subject refractory to conventional therapies for such a disease, said methods comprising administering to said subject a dose of a prophylactically or therapeutically effective amount of an integrin %β₃ antagonist alone or in combination with a dose of a prophylactically or therapeutically effective amount of another therapy (e.g., a prophylactic or therapeutic agent such as those described in Section 4.3 supra). The invention also provides methods for managing, treating or ameliorating chronic otitis media or a symptom thereof in a subject refractory to existing single agent therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides alternative methods for the management or treatment of chronic otitis media or a symptom thereof where conventional therapies have proven or may prove too toxic, i.e., results in unacceptable or unbearable side effects, for the subject being treated. Further, the invention provides methods for preventing the recurrence of chronic otitis media or a symptom thereof in patients that have been treated and have no disease activity by administering a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra).

In each of the foregoing embodiments, a nucleic acid encoding an integrin αᵥβ₃ antagonist may be administered instead of, or in combination with, an integrin αᵥβ₃ antagonist.

4.4.6 Treatment of Hypertrophic Pulmonary Osteoarthropathy

The methods for preventing, managing, treating or ameliorating hypertrophic pulmonary osteoarthropathy or a symptom thereof, said methods comprising administering to a subject in need thereof an integrin αᵥβ₃ antagonist. The present invention also provides methods for preventing, managing, treating or ameliorating hypertrophic pulmonary osteoarthropathy or a symptom thereof, said methods comprising administering to a subject in need thereof an integrin αᵥβ₃ antagonist and at least one therapy other than integrin αᵥβ₃ antagonists (e.g., a prophylactic or therapeutic agent such as those disclosed in Section 4.3 supra).

In a specific embodiment, the invention provides methods for preventing, managing, treating or ameliorating hypertrophic pulmonary osteoarthropathy or a symptom thereof, said methods comprising administering to a subject in need thereof a dose of a prophylactically or therapeutically effective amount of an integrin αᵥβ₃ antagonist and a dose of a prophylactically or therapeutically effective amount of at least one of the following therapies: an anti-inflammatory agent, an immunomodulatory agent, a bone metabolism regulating agent, an antibiotic, hormonal therapy and/or surgery. Preferably, the integrin αᵥβ₃ antagonist is an antibody or antibody fragment that immunospecifically binds to integrin αᵥβ₃. In one embodiment, the antibody is VITAXIN™. In one embodiment, the invention provides a method for preventing, managing, treating or ameliorating hypertrophic pulmonary osteoarthropathy or a symptom thereof, said methods comprising administering to a subject in need thereof a dose of a prophylactically or therapeutically effective amount of an integrin αᵥβ₃ antagonist and a dose of a prophylactically or therapeutically effective amount of at least one chemotherapeutic agent. In another embodiment, the invention provides a method for preventing, managing, treating or ameliorating hypertrophic pulmonary osteoarthropathy or a symptom thereof, said methods comprising administering to a subject in need thereof a dose of a prophylactically or therapeutically effective amount of an integrin αᵥβ₃ antagonist and a dose of a prophylactically or therapeutically effective amount of a bone metabolism regulating agent. In another embodiment, the invention provides a method for preventing, managing, treating or ameliorating hypertrophic pulmonary osteoarthropathy or a symptom thereof, said methods comprising administering to a subject in need thereof a dose of a prophylactically or therapeutically effective amount of an integrin αᵥβ₃ antagonist and a dose of a prophylactically or therapeutically effective amount of an anti-inflammatory agent (e.g., an NSAID). In another embodiment, the invention provides a method for preventing, managing, treating or ameliorating hypertrophic pulmonary osteoarthropathy or a symptom thereof, said methods comprising administering to a subject in need thereof a dose of a prophylactically or therapeutically effective amount of an integrin αᵥβ₃ antagonist and a dose of a prophylactically or therapeutically effective amount of an immunomodulatory agent other than a chemotherapeutic agent.

The compositions and methods of the invention may be used as first, second, third or fourth line of treatment for hypertrophic pulmonary osteoarthropathy or a symptom thereof. The invention provides methods for managing, treating or ameliorating hypertrophic pulmonary osteoarthropathy or a symptom thereof, in a subject refractory to conventional therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides methods for managing, treating or ameliorating hypertrophic pulmonary osteoarthropathy or a symptom thereof in a subject refractory to existing single agent therapies for such a disease, said methods comprising administering to said subject a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra). The invention also provides alternative methods for the management or treatment of hypertrophic pulmonary osteoarthropathy or a symptom thereof where conventional therapies have proven or may prove too toxic, i.e., results in unacceptable or unbearable side effects, for the subject being treated. Further, the invention provides methods for preventing the recurrence of hypertrophic pulmonary osteoarthropathy or a symptom thereof in patients that have been treated and have no disease activity by administering a dose of a prophylactically or therapeutically effective amount of an integrin αᵥβ₃ antagonist alone or in combination with a dose of a prophylactically or therapeutically effective amount of another therapy (e.g., a prophylactic or therapeutic agent such as those described in Section 4.3 supra).

In each of the foregoing embodiments, a nucleic acid encoding an integrin αᵥβ₃ antagonist may be administered instead of, or in combination with, an integrin αᵥβ₃ antagonist.

4.5 Compositions and Methods of Administering Compositions

The present invention provides compositions for the treatment, management prophylaxis, and amelioration of periodontal disease, Gorham-Stout disease, Wilson's disease, chronic otitis media, hypertrophic pulmonary osteoarthropathy or aseptic loosening of joint replacement or a condition or symptom associated therewith, or a symptom thereof. In a specific embodiment, a composition comprises an integrin αᵥβ₃ antagonist. In another embodiment, a composition comprises a nucleic acid molecule encoding an integrin αᵥβ₃ antagonist. In another embodiment, a composition comprises an integrin αᵥβ₃ antagonist and a prophylactic or therapeutic agent other than an integrin αᵥβ₃ antagonist. Preferably, such prophylactic or therapeutic agents are known to be useful for, or have been or are currently being used in the prevention, management, treatment or amelioration of periodontal disease, Gorham-Stout disease, Wilson's disease, chronic otitis media, hypertrophic pulmonary osteoarthropathy or aseptic loosening of joint replacement or a condition or symptom associated therewith. In another embodiment, a composition comprises a nucleic acid molecule encoding an integrin αᵥβ₃ antagonist and at least one prophylactic or therapeutic agent other than an integrin αᵥβ₃ antagonist. Preferably, such prophylactic or therapeutic agents are known to be useful for, or have been or are currently being used in the prevention, treatment or amelioration of periodontal disease, Gorham-Stout disease, Wilson's disease, chronic otitis media, hypertrophic pulmonary osteoarthropathy or aseptic loosening of joint replacement or a condition or symptom associated therewith. In another embodiment, a composition comprises an integrin αᵥβ₃ antagonist and a nucleic acid molecule encoding a prophylactic or therapeutic agent other than an integrin αᵥβ₃ antagonist. Preferably, such prophylactic or therapeutic agents are known to be useful for, or have been or are currently being used in the prevention, treatment or amelioration of periodontal disease, Gorham-Stout disease, Wilson's disease, chronic otitis media, hypertrophic pulmonary osteoarthropathy or aseptic loosening of joint replacement or a condition or symptom associated therewith. In yet another embodiment, a composition comprises a nucleic acid molecule encoding an integrin αᵥβ₃ antagonist and a nucleic acid molecule encoding at least one prophylactic or therapeutic agent other than an integrin αᵥβ₃ antagonist. Preferably, such prophylactic or therapeutic agents are known to be useful for, or have been or are currently being used in the prevention, treatment, management, or amelioration of periodontal disease, Gorham-Stout disease, Wilson's disease, chronic otitis media, hypertrophic pulmonary osteoarthropathy or aseptic loosening of joint replacement or a condition or symptom associated therewith, or a symptom thereof. See Sections 4.3.1 through 4.3.9 for examples of other non-limiting therapeutic or prophylactic agents known to be useful in the prevention, treatment or amelioration of periodontal disease, Gorham-Stout disease, Wilson's disease, chronic otitis media, hypertrophic pulmonary osteoarthropathy or aseptic loosening of joint replacement or a condition or symptom associated therewith.

In a preferred embodiment, a composition of the invention is a pharmaceutical composition. Such compositions comprise a prophylactically or therapeutically effective amount of at least one prophylactic or therapeutic agent (e.g., an integrin αᵥβ₃ antagonist or other prophylactic or therapeutic agent), and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions are sterile and in suitable form for administration to a subject, preferably an animal subject, more preferably a mammalian subject, and most preferably a human subject.

In a specific embodiment, it may be desirable to administer a therapy (e.g., a prophylactic or therapeutic agent) of the invention locally to the area in need of treatment; this may be achieved by, for example, and not by way of limitation, local infusion, by injection, or by means of an implant, said implant being of a porous or non-porous, or gelatinous material, including membranes, such as sialastic membranes matrices, or fibers. Preferably, when administering at least one prophylactic or therapeutic agent, care must be taken to use materials to which the prophylactic or therapeutic agents do not absorb.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, a therapy of the invention can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. No. 5,679,377; U.S. Pat. No. 5,916,597; U.S. Pat. No. 5,912,015; U.S. Pat. No. 5,989,463; U.S. Pat. No. 5,128,326; International Publication No. WO 99/15154; and International Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising at least one prophylactic or therapeutic agent of the invention. See, e.g., U.S. Pat. No. 4,526,938, International publication WO 91/05548, International publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment where the composition of the invention is a nucleic acid molecule encoding a prophylactic or therapeutic agent, such as an antibody of the invention, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868). Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), intratumoral, transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic aqueous buffer. Where necessary, the composition may also include a solubilizing agent and a local anesthetic such as lidocaine to ease pain at the site of the injection.

If the prophylactic or therapeutic agents of the invention are to be administered topically, the prophylactic or therapeutic agents can be formulated in the form of, e.g., a toothpaste, oral gel, ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19^{th} ed., Mack Pub. Co., Easton, Pa. (1995) and Pharmaceutical Dosage Forms and Drug Delivery System, 7th ed., Lippincott Williams & Wilkins (1999). In a particular embodiment, a periodontal disease prophylactic or therapeutic agent of the invention is administered in the form of a toothpaste or oral gel. In another embodiment, a joint disorder prophylactic or therapeutic agent of the invention is administered in the form of an ointment, cream, transdermal patch, lotion, gel, spray, aerosol, solution, or emulsion. For non-sprayable topical dosage forms, viscous to semi-solid or solid forms comprising a carrier or at least one excipient compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

A composition of the invention may be administered intranasally, such as by formulating the agent in an aerosol form, spray, mist or in the form of drops. In particular, a prophylactic or therapeutic agent for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

If the composition of the invention is to be administered orally, a prophylactic or therapeutic agent of the invention can be formulated orally in the form of, e.g., tablets, capsules, cachets, gelcaps, solutions, suspensions and the like. Tablets or capsules can be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose, or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, but are not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates, or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

The composition of the invention may be administered by pulmonary administration, e.g., by use of an inhaler or nebulizer, of a composition formulated with an aerosolizing agent. See, e.g., U.S. Pat. Nos. 6,019,968, 5,985, 320, 5,985,309, 5,934,272, 5,874,064, 5,855,913, 5,290,540, and 4,880,078; and International Publication Nos. WO 92/19244, WO 97/32572, WO 97/44013, WO 98/31346, and WO 99/66903, each of which is incorporated herein by reference their entirety. In a particular embodiment, a method of treating, preventing, managing or ameliorating hypertrophic pulmonary osteoarthropathy or a symptom or condition associated therewith comprises pulmonary administration of at least one prophylactic or therapeutic agent of the invention.

The composition of the invention may be formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The composition of the invention may also be formulated in rectal compositions such as suppositories or retention enemas, e.g., containing conventional suppository bases such as cocoa butter or other glycerides.

In addition to the formulations described previously, the compositions of the invention may also be formulated as a depot preparation. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

In a preferred embodiment, the compositions of the invention are formulated as a toothpaste, topically gel applied to the gums, or a chewable gum for use in preventing, treating, managing, or ameliorating periodontal disease or a symptom thereof.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the invention also provides that a prophylactic or therapeutic agent, or a pharmaceutical composition of the invention is packaged in a hermetically sealed container such as an ampoule or sachette indicating the quantity of the agent. In one embodiment, a prophylactic or therapeutic agent, or pharmaceutical composition of the invention is supplied as a dry sterilized lyophilized powder or water free concentrate in a hermetically sealed container and can be reconstituted, e.g., with water or saline to the appropriate concentration for administration to a subject. Preferably, a the prophylactic or therapeutic agents, or pharmaceutical composition of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agent, or pharmaceutical composition of the invention should be stored at between 2 and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, a prophylactic or therapeutic agent, or pharmaceutical composition of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container. In preferred embodiments of the invention, VITAXIN™ is formulated at 1 mg/ml, 5 mg/ml, 10 mg/ml, and 25 mg/ml for intravenous injections and at 5 mg/ml, 10 mg/ml, 80 mg/ml or 100 mg/ml for repeated subcutaneous administration. In other preferred embodiments of the invention, VITAXIN™ is formulated at 1 mg/ml, 5 mg/ml, 10 mg/ml, 20 mg/ml, 40 mg/ml, 60 mg/ml, 80 mg/ml, or 100 mg/ml for oral administration. In yet other preferred embodiments of the invention, VITAXIN™ is formulated at 5 mg/ml, 10 mg/ml, 15 mg/ml, 20 mg/ml, 50 mg/ml, 100 mg/ml, 200 mg/ml, 300 mg/ml, 400 mg/ml, or 500 mg/ml for topical administration.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. In certain preferred embodiments, the pack or dispenser contains one or more unit dosage forms containing no more than 20 mg Periostat™ and 5 mg/ml VITAXIN™.

Generally, the ingredients of the compositions of the invention are derived from a subject that is the same species origin or species reactivity as recipient of such compositions. Thus, in a preferred embodiment, human or humanized antibodies are administered to a human patient for therapy or prophylaxis.

4.5.1 Gene Therapy

In a specific embodiment, a nucleic acid comprising a sequence encoding a prophylactic or therapeutic agent, is administered to treat, manage, prevent or ameliorate periodontal disease, Gorham-Stout disease, Wilson's disease, chronic otitis media, hypertrophic pulmonary osteoarthropathy or aseptic loosening of joint replacement or a condition associated therewith by way of gene therapy. Gene therapy refers to therapy performed by the administration to a subject of an expressed or expressible nucleic acid. In this embodiment of the invention, the nucleic acids produce their encoded prophylactic or therapeutic agent that mediates a prophylactic or therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises nucleic acids encoding a prophylactic or therapeutic agent, said nucleic acids being part of an expression vector that expresses the prophylactic or therapeutic agent in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the prophylactic or therapeutic agent coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In certain embodiments, the prophylactic or therapeutic agent expressed is an integrin αᵥβ₃ antagonist. In other embodiments the prophylactic or therapeutic agent expressed is an agent known to be useful for, or has been or is currently being used in the prevention, management, treatment or amelioration of aseptic loosening of joint replacement or conditions associated therewith, or periodontal disease, or a symptom thereof. In a preferred embodiment, the prophylactic or therapeutic agent expressed is VITAXIN™.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or by a matrix with in situ scaffolding in which the nucleic acid sequence is contained (see, e.g., European Patent No. EP 0 741 785 B1 and U.S. Pat. No. 5,962,427), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Publication Nos. WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding a prophylactic or therapeutic agent are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110^{-114.}

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include, but are not limited to, epithelial cells; endothelial cells; keratinocytes; fibroblasts; muscle cells; osteoclasts; hepatocytes; blood cells such as T lymphocytes, B lymphocytes, natural killer (NK) cells, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the subject.

In an embodiment in which recombinant cells are used in gene therapy, nucleic acid sequences encoding a prophylactic or therapeutic agent are introduced into the cells such that they are expressible by the cells or their progeny, and the recombinant cells are then administered in vivo for prophylactic or therapeutic effect. In a specific embodiment, stem or progenitor cells are used. Any stem and/or progenitor cells which can be isolated and maintained in vitro can potentially be used in accordance with this embodiment of the present invention (see e.g., International Publication No. WO 94/08598; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises a constitutive, tissue-specific, or inducible promoter operably linked to the coding region. In a preferred embodiment, the nucleic acid to be introduced for purposes of gene therapy comprises an inducible promoter operably linked to the coding region, such that expression of the nucleic acid is controllable by controlling the presence or absence of the appropriate inducer of transcription.

4.5.2 Dosages & Frequency of Administration

The amount of the composition of the invention which will be effective in the treatment, management, prevention or amelioration of periodontal disease, Gorham-Stout disease, Wilson's disease, chronic otitis media, hypertrophic pulmonary osteoarthropathy or aseptic loosening of joint replacement or a condition or symptom associated therewith, can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro and/or animal model test systems. See e.g., a rat periodontitis model described in DeCarlo, A., et al., January 2003, Infect. Immun. 71(1):562-6; and Rajapakse, P. et al., 2002, Infect. Immun. 70(5):2480-6; musk shrew periodontitis model as described in Takata T. et al., 1999, J. Periodontol. 70(2):195-200; Macacafascicularis periodontitis model as described in Persson G. et al., 1994, Oral Microbiol. Immunol. 9(2):104-11; baboon periodontitis model as described in Miller, D. et al., 1995, J Periodontal Res. 30(6):404-9; a rat model of aseptic prosthesis loosening described in Pap, G. et al., 2001, Arthritis Rheum. 44(4):956-63; and a canine model of aseptic loosening described in Dowd, J. et al., 1995, Clin. Orthop. (319):106-21; introduction of ultra-high-molecular-weight polyethylene (UHMWPE) particles to provoke inflammation (Yang, S. Y., et al., 2002, Arthritis Rheum. 46(9):2514-23).

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In a preferred embodiment, the dose of an antibody or antibody fragment that immunospecifically binds to integrin αᵥβ₃ (e.g., VITAXIN® or an antigen-binding fragment thereof) is 0.1 to 10 mg/kg/week, preferably 1 to 9 mg/kg/week, more preferably 2 to 8 mg/week, even more preferably 3 to 7 mg/kg/week, and most preferably 4 to 6 mg/kg/week. In another embodiment, a subject, preferably a human, is administered a dose of a prophylactically or therapeutically effective amount of an antibody or antibody fragment that immunospecifically binds to integrin αᵥβ₃ (e.g., VITAXIN® or an antigen-binding fragment thereof), wherein the dose of a prophylactically or therapeutically effective amount of the antibody or antibody fragment administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses. In another embodiment, a subject, preferably a human, is administered a dose of a prophylactically or therapeutically effective amount of an antibody or antibody fragment that immunospecifically binds to integrin αᵥβ₃ (e.g., VITAXIN™ or an antigen-binding fragment thereof), wherein the dose of a prophylactically or therapeutically effective amount of the antibody or antibody fragment administered to said subject is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

In specific embodiments, an antibody or antibody fragment that immunospecifically binds to integrin αᵥβ₃ (e.g., VITAXIN® or an antigen-binding fragment thereof) is administered in a dosing regimen that maintains the plasma concentration of the antibody immunospecific for integrin αᵥβ₃ at a desirable level (e.g., about 0.1 to about 100 μg/ml), which continuously blocks the integrin αᵥβ₃ activity. In a specific embodiment, the plasma concentration of the antibody is maintained at 0.2 μg/ml, 0.5 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 15 μg/ml, 20 μg/ml, 25 μg/ml, 30 μg/ml, 35 μg/ml, 40 μg/ml, 45 μg/ml or 50 μg/ml. The plasma concentration that is desirable in a subject will vary depending on several factors, including but not limited to, the nature of the disease or disorder, the severity of the disease or disorder and the condition of the subject. Such dosing regimens are especially beneficial in prevention, treatment, management and amelioration of a chronic disease or disorder such as periodontal disease, Gorham-Stout disease, Wilson's disease, chronic otitis media, hypertrophic pulmonary osteoarthropathy or aseptic loosening of joint replacement.

In another embodiment, an antagonist of integrin αᵥβ₃ is administered to a subject with a disease or disorder that associated with bone resportion using a dosing regimen that maintains the plasma concentration of the antagonist at a level that blocks at least 40%, preferably at least 50%, at least 55%, at least 60%, at least 65%, at least 70%, at least 75%, at least 80%, at least 85%, at least 90% or at least 95% of bone resorption relative to a control such as PBS, as measured, for example, by measuring the amount of bone-remodeling or osteolysis by measuring bone volume in vivo by microcomputed tomography analysis or bone histomorphometry analysis.

In specific embodiments, an antibody or antibody fragment that immunospecifically binds to integrin αᵥβ₃ (e.g., in particular, a conjugated antibody or antibody fragment immunospecific for integrin αᵥβ₃) is administered intermittently (e.g., one every other week, once every two weeks, once every three weeks, or once a month). As used herein, “a conjugated antibody or antibody fragment” refers to an antibody or antibody fragment that is conjugated to another moiety, including but not limited to, a heterologous peptide, polypeptide, another antibody or antibody fragment, a marker sequence, a diagnostic agent, a therapeutic agent, a radioactive metal ion, a polymer, albumin, and a solid support.

The dosages of prophylactic or therapeutic agents are described in the Physician's Desk Reference (57th ed., 2003).

4.6 Biological Assays

Several aspects of the pharmaceutical compositions and/or prophylactic or therapeutic agents of the invention are preferably tested in vitro, in a cell culture system, and in an animal model organism, such as a rodent animal model system, for the desired therapeutic activity prior to use in humans. For example, assays which can be used to determine whether administration of a specific pharmaceutical composition is indicated, include cell culture assays in which a patient tissue sample is grown in culture, and exposed to or otherwise contacted with a pharmaceutical composition, and the effect of such composition upon the tissue sample is observed. The tissue sample can be obtained by biopsy from the patient. This test allows the identification of the therapeutically most effective prophylactic or therapeutic molecule(s) for each individual patient. In various specific embodiments, in vitro assays can be carried out with representative cells of cell types involved in periodontal disease, Gorham-Stout disease, Wilson's disease, chronic otitis media, hypertrophic pulmonary osteoarthropathy, aseptic loosening of joint replacement or symptoms or conditions associated therewith (e.g., osteoclasts, B-cells, and activated T-cells) to determine if a pharmaceutical composition of the invention has a desired effect upon such cell types.

The pharmaceutical compositions and combination therapies of the invention can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, the pharmaceutical compositions and combination therapies of the invention are tested in a mouse model system. Such model systems are widely used and well-known to the skilled artisan. Prophylactic and/or therapeutic agents can be administered repeatedly. Several aspects of the procedure may vary. Said aspects include the temporal regime of administering the prophylactic and/or therapeutic agents, and whether such agents are administered separately or as an admixture.

Experimental and spontaneous animal models of periodontal diseases can be used to assess the pharmaceutical compositions and combination therapies of the invention using various experimental animal models of periodontal disease known in the art, for example: a rat periodontitis model described in DeCarlo, A., et al., January 2003, Infect. Immun. 71(1):562-6; and Rajapakse, P. et al., 2002, Infect. Immun. 70(5):2480-6; musk shrew periodontitis model as described in Takata T. et al., 1999, J. Periodontol. 70(2):195-200; Macaca fascicularis periodontitis model as described in Persson G. et al., 1994, Oral Microbiol. Immunol. 9(2):104-11; and baboon periodontitis model as described in Miller, D. et al., 1995, J Periodontal Res. 30(6):404-9; each of which is hereby incorporated by reference in its entirety.

Experimental and spontaneous animal models of aseptic loosening of joint replacement can be used to assess the pharmaceutical compositions and combination therapies of the invention using various experimental animal models of aseptic loosening of joint replacement known in the art, for example: a rat model of aseptic prosthesis loosening described in Pap, G. et al., 2001, Arthritis Rheum. 44(4):956-63; and a canine model of aseptic loosening described in Dowd, J. et al., 1995, Clin. Orthop. (319):106-21. Also known in the art are methods of provoking inflammation and osteolysis to mimic conditions present when aseptic loosening of bone replacement occurs including, but are not limited to, introduction of ultra-high-molecular-weight polyethylene (UHMWPE) particles to provoke inflammation (Yang, S. Y., et al., 2002, Arthritis Rheum. 46(9):2514-23). Each of foregoing references is hereby incorporated by reference in its entirety.

Experimental and spontaneous animal models of chronic otitis media can be used to assess the pharmaceutical compositions and combination therapies of the invention using various experimental animal models of chronic otitis media known in the art, for example: a rat model of chronic otitis media described in Nell et al., 2000, Infect Immun. 68(5): 2992-4. The foregoing reference is hereby incorporated by reference in its entirety.

Experimental and spontaneous animal models of Wilson's disease can be used to assess the pharmaceutical compositions and combination therapies of the invention using various experimental animal models of Wilson's disease known in the art, for example: the Long-Evans Cinnamon rat model of Wilson's disease described in Terada and Sugiyama, 1999, Pediatr Int. 41(4): 414-8. The foregoing reference is hereby incorporated by reference in its entirety.

Experimental and spontaneous animal models can also be used to assess the anti-inflammatory activity of the pharmaceutical compositions and combination therapies of the invention which may be used to prevent, treat, manage or ameliorate inflammation associated with periodontal disease, Gorham-Stout disease, Wilson's disease, chronic otitis media, hypertrophic pulmonary osteoarthropathy or aseptic loosening of joint replacement (prostheses). The following are some assays provided as examples and not by limitation.

The anti-inflammatory activity of the pharmaceutical compositions and combination therapies of the invention can be assessed using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al., “Carrageenan-Induced Arthritis in the Rat,” Inflammation, 24(2): 141-155, (2000). Also commonly used are zymosan-induced inflammation animal models as known and described in the art.

The anti-inflammatory activity of the pharmaceutical compositions and combination therapies of the invention can also be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for the anti-inflammatory activity of most NSAIDs, and is considered predictive of human efficacy. The anti-inflammatory activity of the test prophylactic or therapeutic agents is expressed as the percent inhibition of the increase in hind paw weight of the test group relative to the vehicle dosed control group.

In a specific embodiment of the invention where the experimental animal model used is adjuvant-induced arthritis rat model, body weight can be measured relative to a control group to determine the anti-inflammatory activity of the combination therapies of invention. Combination therapies tested may include, but are not limited to, combinations comprising any integrin αᵥβ₃ antagonist functionally homologous to VITAXIN™ may also be tested in combination therapies in rat models.

Current methods known in the art of measuring the progression or regression of Gorham-Stout disease include analyzing the circulating number of osteoclast precursors and sensitivity to osteoclastogenic factors in a Gorham-Stout disease patient. Monocytes may be cultured with M-CSF (25 ng/ml) and RANKL (30 ng/ml) and osteoclast formation may be assessed in terms of the formation of TRAP(+) and VNR(+) multinucleated cells and the extent of lacunar resorption. See Hirayama et al., 2001, J Pathol. 195(5): 624-30, incorporated by reference in its entirety.

Current methods known in the art of measuring the progression or regression of periodontal disease include, but are not limited to, measurements of periodontal bone and attachment loss, including digital subtraction radiology, Jeffcoat, M. and Reddy, M., 2000, Monogr. Oral Sci. 17:56-72.

The effect of the pharmaceutical compositions and combination therapies of the invention on periodontal disease can be monitored/assessed using standard techniques known to one of skill in the part. Periodontal disease in a subject can be determined by, e.g., measuring the extent of gingival inflammation and bleeding, the probing depth of the pocket to the point of resistance (e.g., one or more sites with a depth of at least 5 mm), the clinical attachment loss of the periodontal ligament measured from a fixed point of the tooth (usually the cemento-enamel junction), the loss of adjacent alveolar bone as measured by x-ray (e.g., periapical or bitewing dental radiographs), and plaque accumulation. See U.S. Pat. No. 6,277,587.

Current methods known in the art of measuring the progression or regression of periodontal disease, Gorham-Stout disease, chronic otitis media, hypertrophic osteoarthropathy, and aseptic loosening of joint replacement include but are not limited to measurement of bone-remodeling, osteolysis, and linear/volumetric wear. Kim, Y. H. et al., 2003, J. Bone Joint Surg. Am 85-A(1):109-14. This may be done by using radiological, sonographical, magnetic resonance imaging (MRI) methods including digital subtraction radiology (Jeffcoat, M. and Reddy, M., 2000, Monogr. Oral Sci. 17:56-72; Schill, S. et al., 2002, Orthopade 31(12):1132-44). Other methods for measuring aseptic loosening of hip replacement which are well-established in the art include the Harris hip score (Soerman, P., and Malchau, H., 2001, Clin. Ortho. Related Res. 384:189-197); the Merle d'Aubign and Postel hip score, the McMaster-Toronto Arthritis Patient Preference Disability Questionnaire (MACTAR), and the Western Ontario and McMaster University Osteoarthritis Index (WOMAC) (Laupacis A. et al., 2002, J. Bone Joint Surg. Am. 84-A(10): 1823-8); and others (Andersson, G., 1972, J. Bone Joint Surg. Br. 54(4):621-5); each of the above references is hereby incorporated by reference in its entirety.

The efficacy of the pharmaceutical compositions and combination therapies of the invention can also be assessed using assays that determine bone loss as a measure of periodontal disease, Gorham-Stout disease, chronic otitis media, hypertrophic pulmonary osteoarthropathy or aseptic loosening ofjoint replacement (prostheses). Animal models such as ovariectomy-induced bone resorption mice, rat and rabbit models are known in the art for obtaining dynamic parameters for bone formation. Using methods such as those described by Yositake et al. or Yamamoto et al., bone volume is measured in vivo by microcomputed tomography analysis and bone histomorphometry analysis. Yoshitake et al., “Osteopontin-Deficient Mice Are Resistant to Ovariectomy-Induced Bone Resorption,” Proc. Natl. Acad. Sci. 96:8156-8160, (1999); Yamamoto et al., “The Integrin Ligand Echistatin Prevents Bone Loss in Ovariectomized Mice and Rats,” Endocrinology 139(3):1411-1419, (1998), both incorporated herein by reference in their entirety.

Further, any assays known to those skilled in the art can be used to evaluate the prophylactic and/or therapeutic utility of the pharmaceutical compositions and combination therapies of the invention disclosed herein for periodontal disease, Gorham-Stout disease, Wilson's disease, chronic otitis media, hypertrophic pulmonary osteoarthropathy or aseptic loosening of joint replacement (prostheses), or conditions associated therewith.

The toxicity and/or efficacy of the prophylactic and/or therapeutic protocols of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Pharmaceutical compositions and combination therapies that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

4.7 Detection & Diagnosis

The compositions of the invention comprising labeled integrin αᵥβ₃ antagonists (e.g., antibodies that immuospecifically binds to integrin αᵥβ₃) can be used for diagnostic purposes to detect, diagnose, or monitor periodontal disease, Wilson's disease, Gorham-Stout disease, hypertrophic pulmonary osteoarthropathy, chronic otitis media, or aseptic loosening of joint replacement or a condition or symptom associated therewith, including inflammation or bone resorption due to each of the foregoing. In a preferred embodiment, antibodies that immunospecifically bind to integrin αᵥβ₃ are used for diagnostic purposes to detect, diagnosis, or monitor periodontal disease, Wilson's disease, Gorham-Stout disease, hypertrophic pulmonary osteoarthropathy, chronic otitis media, or aseptic loosening of joint replacement or a condition or symptom associated therewith. The detection or diagnosis of periodontal disease, Wilson's disease, Gorham-Stout disease, hypertrophic pulmonary osteoarthropathy, chronic otitis media, or aseptic loosening of joint replacement or a condition or symptom associated therewith can be conducted utilizing an effective amount (i.e., an amount effective to be able to detect the expression of integrin αᵥβ₃) of a composition of the invention in an in vitro or in vivo assay using techniques well-known to one of skilled in the art. In a preferred embodiment, aseptic loosening of joint replacement or conditions associated therewith, or periodontal disease or symptoms thereof, is detected in the subject, preferably a mammalian subject and most preferably a human subject utilizing an effective amount of a composition of the invention in a standard imaging technique known to one of skilled in the art.

The invention encompasses methods of detecting, diagnosing, or monitoring periodontal disease, Wilson's disease, Gorham-Stout disease, hypertrophic pulmonary osteoarthropathy, chronic otitis media, or aseptic loosening of joint replacement or a condition or symptom associated therewith, said methods comprising: a) administering to a subject an effective amount of a composition of the invention comprising a labeled antibody or antibody fragment that immunospecifically binds to integrin αᵥβ₃; b) waiting for a time interval following the administering for permitting the labeled antibody or antibody fragment to preferentially concentrate at any desired site, e.g., an aseptic loosened joint, in the subject (and for unbound labeled antibody or antibody fragment to be cleared to background level); c) determining background level; and d) detecting the labeled molecule in the subject, such that detection of labeled antibody or antibody fragment above the background level indicates the presence of the disease.

The invention encompasses methods of detecting, diagnosing or monitoring periodontal disease, Wilson's disease, Gorham-Stout disease, hypertrophic pulmonary osteoarthropathy, chronic otitis media, or aseptic loosening of joint replacement or a condition or symptom associated therewith, said methods comprising: a) administering to a subject an effective amount of a composition of the invention comprising an antibody or antibody fragment that immunospecifically binds to integrin αᵥβ₃; b) administering a second labeled antibody or antibody fragment that recognizing the antibody or antibody fragment of the composition of the invention; c) waiting for a time interval following the administering for permitting the labeled antibody or antibody fragment to preferentially concentrate at any desired site, e.g., an aseptic loosened joint, in the subject (and for unbound labeled antibody or antibody fragment to be cleared to background level); d) determining background level; and e) detecting the labeled molecule in the subject, such that detection of labeled antibody or antibody fragment above the background level indicates the presence of the disease.

The invention encompasses methods for diagnosing, detecting, or monitoring periodontal disease, Wilson's disease, Gorham-Stout disease, hypertrophic pulmonary osteoarthropathy, chronic otitis media, or aseptic loosening of joint replacement or a condition or symptom associated therewith, said methods comprising imaging said subject at a time interval after administering to said subject an effective amount of a composition of the invention comprising a labeled antibody or antibody fragment that immunospecifically binds to integrin αᵥβ₃, said time interval being sufficient to permit the labeled antibody or antibody fragment to preferentially concentrate at a specific site, e.g., an aspetic loosened joint, in said subject, wherein detection of the labeled antibody or antibody fragment localized at the site in the subject indicates the presence of the disease or disorder.

In some embodiments, monitoring of a disease or disorder is carried out by repeating the method for diagnosing the disease or disorder, for example, one month after initial diagnosis, six month after initial diagnosis, and one year afer initial diagnosis.

4.7.1 Methods of Detection and Imaging

Presence of the integrin αᵥβ₃ antagonist can be detected in the patient using methods known in the art for in vivo scanning. These methods depend upon the type of label used. Skilled artisans will be able to determine the appropriate method for detecting a particular label. Methods and devices that may be used in the diagnostic methods of the invention include but are not limited to: computed tomography (CT), whole body scan such as position emission tomography (PET), magnetic resonance imaging (MRI), and sonography.

In a specific embodiment, the integrin αᵥβ₃ antagonist is labeled with a radioisotope and is detected in the patient using a radiation responsive surgical instrument (Thurston et al., U.S. Pat. No. 5,441,050). In another embodiment, the integrin αᵥβ₃ antagonist is labeled with a fluorescent compound and is detected in the patient using a fluorescence responsive scanning instrument. In another embodiment, the integrin αᵥβ₃ antagonist is labeled with a positron emitting metal and is detected in the patent using positron emission-tomography. In yet another embodiment, the integrin αᵥβ₃ antagonist is labeled with a paramagnetic label and is detected in a patient using magnetic resonance imaging (MRI).

4.8 Articles of Manufacture

The present invention also encompasses a finished packaged and labeled pharmaceutical product. The present invention provides articles of manufacture comprising packaging material and a pharmaceutical composition of the invention in suitable form for administration to a subject contained within said packaging material. In particular, the present invention provides article of manufactures comprising packaging material and a pharmaceutical composition of the invention in suitable form for administration to a subject contained within said packaging material wherein, said pharmaceutical composition comprises an integrin αᵥβ₃ antagonist, and a pharmaceutically acceptable carrier. The present invention also provides articles of manufacture comprises packaging material and instructions and two pharmaceutical compositions in suitable form for administration to a human contained within said packaging material, wherein the first pharmaceutical composition comprises an integrin αᵥβ₃ antagonist and a pharmaceutically acceptable carrier, and the second pharmaceutical composition comprises a prophylactic or therapeutic agent other than an integrin αᵥβ₃ antagonist and a pharmaceutically acceptable carrier. The present invention also provides articles of manufacture comprising packaging material and a pharmaceutical composition of the invention in suitable form for administration to a subject contained within said packaging material wherein, said pharmaceutical composition comprises an integrin αᵥβ₃ antagonist, at least one prophylactic or therapeutic agent other than an integrin αᵥβ₃ antagonist, and a pharmaceutically acceptable carrier.

In a specific embodiment, an article of manufacture comprises packaging material and a pharmaceutical composition in suitable form for administration to a subject and instructions contained within said packaging material, wherein said pharmaceutical composition comprises an antibody that immunospecifically binds to integrin αᵥβ₃ and a pharmaceutically acceptable carrier, and said instructions suggest a dosing regimen for the treatment, management or amelioration of periodontal disease, Wilson's disease, Gorham-Stout disease, hypertrophic pulmonary osteoarthropathy, chronic otitis media, or aseptic loosening of joint replacement or a condition or symptom associated therewith. In another embodiment, the article of manufacture comprises packaging material and instructions and two pharmaceutical compositions in suitable form for administration to a human contained within said packaging material, wherein the first pharmaceutical composition comprises an antibody that immunospecifically binds to integrin αᵥβ₃ and a pharmaceutically acceptable carrier, and the second pharmaceutical composition comprises a prophylactic or therapeutic agent other than an integrin αᵥβ₃ antagonist and a pharmaceutically acceptable carrier, and said instructions suggest a dosing regimen for the treatment, management or amelioration of periodontal disease, Wilson's disease, Gorham-Stout disease, hypertrophic pulmonary osteoarthropathy, chronic otitis media, or aseptic loosening of joint replacement or a condition or symptom associated therewith. In another embodiment, an article of manufacture comprises packaging material and a pharmaceutical composition in suitable form for administration to a subject and instructions contained within said packaging material, wherein said pharmaceutical composition comprises an antibody that immunospecifically binds to integrin αᵥβ₃, at least one prophylactic or therapeutic agent other than an integrin αᵥβ₃ antagonist, and a pharmaceutically acceptable carrier, and said instructions suggest a dosing regimen for the treatment, management or amelioration of periodontal disease, Wilson's disease, Gorham-Stout disease, hypertrophic pulmonary osteoarthropathy, chronic otitis media, or aseptic loosening of joint replacement or a condition or symptom associated therewith.

In another embodiment, an article of manufacture comprises packaging material and a pharmaceutical composition in suitable form for administration to a human and instructions contained within said packaging material, wherein said pharmaceutical composition comprises VITAXIN™ or an antigen-binding fragment thereof, and a pharmaceutically acceptable carrier, and said instructions suggest a dosing regimen for the treatment, management or amelioration of periodontal disease, Wilson's disease, Gorham-Stout disease, hypertrophic pulmonary osteoarthropathy, chronic otitis media, or aseptic loosening of joint replacement or a condition or symptom associated therewith. In another embodiment, an article of manufacture comprises packaging material and instructions and two pharmaceutical compositions in suitable form for administration to a human and instructions contained within said packaging material, wherein the first pharmaceutical composition comprises an VITAXIN™ and a pharmaceutically acceptable carrier, and the second pharmaceutical composition comprises a prophylactic or therapeutic agent other than an integrin αᵥβ₃ antagonist and a pharmaceutically acceptable carrier, and said instructions suggest a dosing regimen for the treatment, management or amelioration of periodontal disease, Wilson's disease, Gorham-Stout disease, hypertrophic pulmonary osteoarthropathy, chronic otitis media, or aseptic loosening of joint replacement or a condition or symptom associated therewith. In a preferred embodiment, an article of manufacture comprises packaging material and a pharmaceutical composition in suitable form for administration to a human and instructions contained within said packaging material, wherein said pharmaceutical composition comprises VITAXIN™ or an antigen-binding fragment thereof, at least one prophylactic or therapeutic agent other than an integrin αᵥβ₃ antagonist, and a pharmaceutically acceptable carrier, and said instructions suggest a dosing regimen for the treatment, management or amelioration of periodontal disease, Wilson's disease, Gorham-Stout disease, hypertrophic pulmonary osteoarthropathy, chronic otitis media, or aseptic loosening of joint replacement or a condition or symptom associated therewith.

As with any pharmaceutical product, the packaging material and container of the articles of manufacture of the invention are designed to protect the stability of the product during storage and shipment. More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material. The invention also encompasses the use of an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery. The invention encompasses solutions, preferably sterile, suitable for each delivery route.

The articles of manufacture of the invention may include instructions regarding the use or administration of a pharmaceutical composition, or other informational material that advises the physician, technician or patient on how to appropriately prevent or treat the disease or disorder in question. In other words, the article of manufacture includes instruction means indicating or suggesting a dosing regimen including, but not limited to, actual doses and monitoring procedures.

The present invention provides that the adverse effects that may be reduced or avoided by the methods of the invention are indicated in informational material enclosed in an article of manufacture for use in preventing, treating or ameliorating a symptom associated with aseptic loosening of joint replacement or periodontal disease. Adverse effects that may be reduced or avoided by the methods of the invention include but are not limited to vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation. Since some of the prophylactic or therapeutic agents used in the accordance with the invention may be immunosuppressive, prolonged immunosuppression may increase the risk of infection, including opportunistic infections. Prolonged and sustained immunosuppression may also result in an increased risk of developing certain types of cancer.

Further, the information material enclosed in an article of manufacture for use in preventing, treating, managing or ameliorating a symptom or condition associated with periodontal disease, Gorham-Stout disease, Wilson's disease, chronic otitis media, hypertrophic pulmonary osteoarthropathy or aseptic loosening of joint replacement can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactionsincluding anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

4.9 Methods for Producing Antibodies

Antibodies that immunospecifically bind to an antigen (e.g., integrin αᵥβ₃) can be produced by any method known in the art for the synthesis of antibodies, in particular, by chemical synthesis or preferably, by recombinant expression techniques.

Polyclonal antibodies that immunospecifically bind to an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681, Elsevier, N.Y., (1981) and Harlow et al., Using Antibodies: A Laboratory Manual, Cold Spring Harbor Laboratory Press (1999) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen (e.g., integrin αᵥβ₃) and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC™. Hybridomas are selected and cloned by limited dilution. Additionally, a RIMMS (repetitive immunization multiple sites) technique can be used to immunize an animal (Kilptrack et al., 1997, Hydridoma 16: 381-9, incorporated by reference in its entirety). The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating antibodies by culturing a hybridoma cell secreting an antibody of the invention wherein, preferably, the hybridoma is generated by fusing splenocytes isolated from a mouse immunized with a non-murine antigen (e.g., integrin αᵥβ₃) with myeloma cells and then screening the hybridomas resulting from the fusion for hybridoma clones that secrete an antibody able to bind to the antigen (e.g., integrin αᵥβ₃)

Antibody fragments which recognize specific particular epitopes (e.g., integrin αᵥβ₃) may be generated by any technique known to those of skill in the art. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International Application No. PCT/GB91/O1134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, 5,969,108, 6,333,187, 5,824,520, 5,702,892; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in International publication No. WO 92/22324; Mullinax et al., 1992, BioTechniques 12(6):864-869; Sawai et al., 1995, AJR134:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1 a promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized, human, or chimeric antibodies. Completely human antibodies and humanized antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immuoglobulin. A humanized antibody comprises substantially all of at least one, and typically two, variable domains (Fab, Fab′, F(ab′)₂, Fabc, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and most preferably greater than 95%. Humanized antibody can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119-25 (2002), Caldas et al., Protein Eng. 13(5):353-60 (2000), Morea et al., Methods 20(3):267-79 (2000), Baca et al., J. Biol. Chem. 272(16):10678-84 (1997), Roguska et al., Protein Eng. 9(10):895-904 (1996), Couto et al., Cancer Res. 55 (23 Supp):5973s-5977s (1995), Couto et al., Cancer Res. 55(8):1717-22 (1995), Sandhu JS, Gene 150(2):409-10 (1994), and Pedersen et al., J. Mol. Biol. 235(3):959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.) Single domain antibodies, for example, antibodies lacking the light chains, can be produced by methods well-known in the art. See Riechmann et al., 1999, J. Immuno. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety.

Further, the antibodies that immunospecifically bind to an antigen (e.g., integrin αᵥβ₃) can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

4.9.1 Polynucleotide Sequences Encoding an Antibody

The invention provides polynucleotides comprising a nucleotide sequence encoding an antibody or antibody fragment that immunospecifically binds to an antigen (e.g., integrin αᵥβ₃). The invention also encompasses polynucleotides that hybridize under high stringency, intermediate or lower stringency hybridization conditions, e.g., as defined supra, to polynucleotides that encode an antibody of the invention.

The polynucleotides may be obtained, and the nucleotide sequence of the polynucleotides determined, by any method known in the art. The nucleotide sequence of antibodies immunospecific for a desired antigen can be obtained, e.g., from the literature or a database such as GenBank. Since the amino acid sequences of VITAXIN™ is known, nucleotide sequences encoding these antibodies can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, or variants thereof, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, at least one of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide sequence generated by the combination of the framework regions and CDRs encodes an antibody that immunospecifically binds to a particular antigen (e.g., integrin αᵥβ₃). Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

4.9.2 Recombinant Expression of Antibodies

Recombinant expression of an antibody of the invention (e.g., a heavy or light chain of an antibody of the invention or a single chain antibody of the invention) that immunospecifically binds to an antigen (e.g., integrin αᵥβ₃) requires construction of an expression vector containing a polynucleotide that encodes the antibody. Once a polynucleotide encoding an antibody molecule, heavy or light chain of an antibody, or antibody fragment (preferably, but not necessarily, containing the heavy or light chain variable domain) of the invention has been obtained, the vector for the production of the antibody molecule may be produced by recombinant DNA technology using techniques well-known in the art. See, e.g., U.S. Pat. No. 6,331,415, which is incorporated herein by reference in its entirety. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., International Publication WO 86/05807; International Publication No. WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45: 101; and Cockett et al., 1990, Bio/Technology 8:2). In a specific embodiment, the expression of nucleotide sequences encoding antibodies of the invention, derivatives or analogs thereof which immunospecifically bind to at least one antigen (e.g., integrin αᵥβ₃) is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7030 and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoters, enhancers, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150: 1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

The present invention also encompasses proteins, peptides and polypeptides of the invention produced by any method known in the art including, but not limited to, recombinant methods. See International Publication No. WO 02/070007, hereby incorporated by reference in its entirety.

4.10 Equivalents

The present invention is not to be limited in scope by the exemplified embodiments, which are intended as illustrations of single aspects of the invention. Indeed, various modifications of the invention in addition to those shown and described herein will become apparent to those skilled in the art from the foregoing description. Such modifications are intended to fall within the scope of the appended claims.

All patents, patent applications and non-patent publications cited herein are incorporated by reference in their entirety to the same extent as if each individual patent, patent application or non-patent publication was specifically and individually indicated to be incorporated herein by reference.

What is claimed is:

1. A method of treating, managing or ameliorating a disease or a symptom or condition associated therewith, said method comprising administering to a subject in need thereof an effective amount of an integrin αᵥβ₃ antagonist, wherein the disease is aseptic loosening of a joint replacement, periodontal disease, Gorham-Stout disease, Wilson's disease, chronic otitis media, or hypertrophic pulmonary osteoarthropathy.

2. A method of treating, managing or ameliorating a disease or a symptom or condition associated therewith, said method comprising the steps of:
a) administering to a subject in need thereof an effective amount of an integrin αᵥβ₃ antagonist; and
b) administering to said subject a therapeutically effective amount of a therapeutic agent other than an integrin αᵥβ₃ antagonist,
wherein the disease is aseptic loosening of a joint replacement, periodontal disease, Gorham-Stout disease, Wilson's disease, chronic otitis media, or hypertrophic pulmonary osteoarthropathy.

3. The method of claim 1, wherein the disease is aseptic loosening of a joint replacement.

4. The method of claim 2, wherein the disease is aseptic loosening of a joint replacement.

5. The method of claim 1, wherein the disease is periodontal disease.

6. The method of claim 2, wherein the disease is periodontal disease.

7. The method of claim 1, wherein the disease is Gorham-Stout disease.

8. The method of claim 2, wherein the disease is Gorham-Stout disease.

9. The method of claim 1, wherein the disease is Wilson's disease.

10. The method of claim 2, wherein the disease is Wilson's disease.

11. The method of claim 1, wherein the disease is chronic otitis media.

12. The method of claim 2, wherein the disease is chronic otitis media.

13. The method of claim 1, wherein the disease is hypertrophic pulmonary osteoarthropathy.

14. The method of claim 2, wherein the disease is hypertrophic pulmonary osteoarthropathy.

15. The method of claim 1 or 2 further comprising conducting surgery on said subject.

16. The method of claim 5 or 6 further comprising conducting dental surgery on said subject.

17. The method of claim 4, 6, 8, 12 or 14, wherein the therapeutic agent is a bone metabolism regulating agent.

18. The method of claim 17, wherein the bone metabolism regulating agent is a bisphosphonate or a vitamin D compound.

19. The method of claim 2, wherein the therapeutic agent is an analgesic, immunomodulatory agent, or anti-inflammatory agent.

20. The method of claim 19, wherein the analgesic is a non-steroidal anti-inflammatory drug, salicylate, or acetominophen.

21. The method of claim 4, 6, 12 or 14, wherein the therapeutic agent is an antibiotic.

22. The method of claim 21, wherein the antibiotic is penicillin, tetracycline, streptomycin, gentamycin, or neomycin.

23. The method of claim 19, wherein the therapeutic agent is an anti-inflammatory agent.

24. The method of claim 23, wherein the anti-inflammatory agent is a non-steroidal anti-inflammatory drug.

25. The method of claim 1 or 2, wherein the integrin αᵥβ₃ antagonist is administered parenterally.

26. The method of claim 1 or 2, wherein the integrin αᵥβ₃ antagonist is administered orally or topically.

27. The method of claim 5 or 6, wherein the integrin αᵥβ₃ antagonist is administered as a toothpaste or a gel that is applied to the gums.

28. The method of claim 5 or 6, wherein the periodontal disease is gingivitis, periodontitis, or periodontosis.

29. The method of claim 1 or 2, wherein the integrin αᵥβ₃ antagonist is an antibody or antibody fragment that immunospecifically binds to integrin αᵥβ₃.

30. The method of claim 1 or 2, wherein the integrin αᵥβ₃ antagonist is an antibody or antibody fragment is conjugated to a therapeutic moiety.

31. The method of claim 1 or 2, wherein the integrin αᵥβ₃ antagonist is VITAXIN™ or an antigen-binding fragment thereof.

32. The method of claim 1 or 2, wherein the integrin αᵥβ₃ antagonist is VITAXIN™ or an antigen-binding fragment thereof conjugated to a therapeutic moiety.

33. The method of claim 1 or 2, wherein the subject is human.

34. An article of manufacture comprising packaging material, instructions, and a pharmaceutical composition in suitable form for administration to a human, contained within said packaging material, wherein said pharmaceutical composition comprises an integrin αᵥβ₃ antagonist and a pharmaceutically acceptable carrier, and said instructions suggest a dosing regimen for the prevention, treatment, management, or amelioration of a disease or a symptom or condition associated therewith, wherein the disease is aseptic loosening of a joint replacement, periodontal disease, Gorham-Stout disease, Wilson's disease, chronic otitis media, or hypertrophic pulmonary osteoarthropathy.

35. An article of manufacture comprising packaging material, instructions, and two pharmaceutical compositions in suitable form for administration to a human contained within said packaging material, wherein the first pharmaceutical composition comprises an integrin αᵥβ₃ antagonist and a pharmaceutically acceptable carrier, and the second pharmaceutical composition comprises a prophylactic or therapeutic agent other than an integrin αᵥβ₃ antagonist and a pharmaceutically acceptable carrier, and said instructions suggest a dosing regimen for the prevention, treatment, management, or amelioration of a disease or a symptom or condition associated therewith, wherein the disease is aseptic loosening of a joint replacement, periodontal disease, Gorham-Stout disease, Wilson's disease, chronic otitis media, or hypertrophic pulmonary osteoarthropathy.

36. The article of manufacture of claim 34 or 35, wherein the integrin αᵥβ₃ antagonist is an antibody or antibody fragment that immunospecifically binds to integrin αᵥβ₃.

37. The article of manufacture of claim 36, wherein the antibody or antibody fragment is conjugated to a therapeutic moiety.

38. The article of manufacture of claim 34 or 35, wherein the integrin αᵥβ₃ antagonist is VITAXIN™ or an antigen-binding fragment thereof.

39. The article of manufacture of claim 34 or 35, wherein the integrin αᵥβ₃ antagonist is VITAXIN™ or an antigen-binding fragment thereof conjugated to a therapeutic moiety.

40. The article of manufacture of claim 35, wherein the prophylactic or therapeutic agent other than an integrin αᵥβ₃ antagonist is an analgesic, an anti-inflammatory agent, or an immunomodulatory agent.


SEQUENCE LISTING (6 sequences)

SEQ ID NO: 1 - protein, 5 aa, Murine
  VH1 CDR of LM609
SYDMS

SEQ ID NO: 2 - protein, 7 aa, Murine
  VH2 CDR of LM609
KVSSGGG

SEQ ID NO: 3 - protein, 8 aa, Murine
  VH3 CDR of LM609
HNYGSFAY

SEQ ID NO: 4 - protein, 11 aa, Murine
  VL1 CDR of LM609
QASQSISNHLH

SEQ ID NO: 5 - protein, 7 aa, Murine
  VL2 CDR of LM609
YRSQSIS

SEQ ID NO: 6 - protein, 9 aa, Murine
  VL3 CDR of LM609
QQSGSWPHT